(19) United States
(12) Patent Application Publication
(10) Pub. No.: US 2024/0000912 A1
(43) Pub. Date: Jan. 4, 2024

(54) IMMUNOGENIC COMPOSITIONS FOR USE IN PNEUMOCOCCAL VACCINES

(71) Applicant: Pfizer Inc., New York, NY (US)

(72) Inventors: Annaliesa Sybil Anderson, Upper Saddle River, NJ (US); Seema Shridhar Gangolli, Park Ridge, NJ (US); Kathrin Ute Jansen, New York, NY (US); Avvari Krishna Prasad, Chapel Hill, NC (US); Michael William Pride, Staten Island, NY (US); Ingrid Lea Scully, Cornwall, NY (US); Wendy Jo Watson, Blue Bell, PA (US)

(21) Appl. No.: 18/034,239

(22) PCT Filed: Nov. 1, 2021
(86) PCT No.: PCT/IB2021/060097

§ 371 Date: Apr. 27, 2023

Related U.S. Application Data
(60) Provisional application No. 63/109,423, filed on Nov. 4, 2020.

(51) Int. Cl. A61K 39/09; A61P 31/04

(52) U.S. Cl. CPC A61K 39/092; A61P 31/04; A61K 2039/70

(57) ABSTRACT

An object of the present invention is to provide immunogenic compositions for protection against S. pneumoniae, in particular against S. pneumoniae serogroup 15, while limiting the number of conjugates. The present invention therefore relates to new immunogenic compositions for use in pneumococcal vaccines and to vaccination of human subjects, in particular infants and elderly, against pneumococcal infections using said immunogenic compositions.

### FIELD OF THE INVENTION

An object of the present invention is to provide immunogenic compositions for protection against S. pneumoniae serogroup 15. Immunogenic compositions of the present invention will typically comprise conjugated capsular saccharide antigens (glycoconjugates), wherein the saccharides are derived from serotypes of Streptococcus pneumoniae. The present invention relates to new immunogenic compositions for use in pneumococcal vaccines.

### BACKGROUND OF THE INVENTION

Infections caused by pneumococci are a major cause of morbidity and mortality all over the world. Pneumonia, febrile bacteraemia and meningitis are the most common manifestations of invasive pneumococcal disease, whereas bacterial spread within the respiratory tract may result in middle-ear infection, sinusitis or recurrent bronchitis. Compared with invasive disease, the non-invasive manifestations are usually less severe, but considerably more common.

The etiological agent of pneumococcal diseases, Streptococcus pneumoniae (pneumococcus), is a Gram-positive encapsulated coccus, surrounded by a polysaccharide capsule. Differences in the composition of this capsule permit serological differentiation between about 91 capsular types, some of which are frequently associated with pneumococcal disease, others rarely. Invasive pneumococcal infections include pneumonia, meningitis and febrile bacteremia; among the common non-invasive manifestations are otitis media, sinusitis and bronchitis.

Pneumococcal conjugate vaccines (PCVs) are pneumococcal vaccines used to protect against disease caused by S. pneumoniae (pneumococcus). There are currently three PCV vaccines available on the global market: PREVNAR® (called Prevenar in some countries) (heptavalent vaccine), SYNFLORIX® (a decavalent vaccine) and PREVNAR 13® (tridecavalent vaccine).

The specific serotypes causing disease beyond the 13 in PREVNAR 13® vary by region, population, and may change over time due to acquisition of antibiotic resistance, pneumococcal vaccine introduction and secular trends of unknown origin.

The addition of conjugates to an immunogenic composition is not a straightforward process as the combination of conjugates into a single multivalent injection may result in competition among the different components and may adversely affect the immunogenicity of any individual conjugate.

This phenomenon of interference may limit the number of conjugates which can be included in a multi-valent vaccine. Therefore, protection against a high number of serotypes, while limiting the number of conjugates in the composition, maybe very difficult to obtain despite of the significant value.

S. pneumoniae serogroup 15 comprises four serotypes: 15A, 15B, 15C and 15F.

Serotypes 15A, 15B and 15C are of particular concern where 15F seems to be of less medical concern (see e.g. Cui Y A et al. Hum Vaccin Immunother. 2017; 13(6):1-13).

An object of the present invention is to provide immunogenic compositions or vaccine schedule for appropriate protection against S. pneumoniae, in particular against S. pneumoniae serotypes 15A, 15B and 15C while limiting the number of conjugates.

### SUMMARY OF THE INVENTION

WO2019/139692 has tested cross protection across the serogroup 15 (see e.g. Example 42 pages 133-134). WO2019/139692 discloses that immunization with serogroups 15A, 15B or 15C monovalent pneumococcal conjugate vaccines had equivalent post dose 2 IgG and OPA titers to the homologous and heterologous polysaccharide and bacterial strain, respectively. WO2019/139692 discloses that rabbits immunized with 15A conjugate, 15B conjugate or 15C conjugate all had cross reactivity to each pneumococcal polysaccharide (15A, 15B and 15C) (see FIG. 17). WO2019/139692 concludes that there was no significant difference in the IgG titers across serogroup 15 and that rabbits immunized with 15A, 15B or 15C conjugate all had cross reactivity to each S. pneumoniae bacterial strain, (15A, 15B, 15C), as all rabbit hyper immune sera had functional antibody to each strain evaluated and killed the bacteria (see FIG. 18 and page 134 lines 9-13). WO2019/139692 found no significant difference in OPA titers across the serogroup 15. Contrary to what WO2019/139692 suggests, it has been surprisingly found that in human, while serotype 15B polysaccharide conjugate beyond eliciting functional reactive antibodies to serogroup 15B, can additionally elicit strong cross-functional responses against serotype 15C isolates, but only a minor cross-functional response against serotype 15A.

Therefore, in order to achieve optimal protection against S. pneumoniae serotypes 15A, 15B and 15C, it is preferable to immunize a subject with both a serotype 15B polysaccharide conjugate and a serotype 15A polysaccharide conjugate, or with both a serotype 15C polysaccharide conjugate and a serotype 15A polysaccharide conjugate.

In order to achieve protection against S. pneumoniae serotypes 15A, 15B and 15C, it is also possible to immunize a subject with a serotype 15B polysaccharide conjugate, a serotype 15C polysaccharide conjugate and a serotype 15A polysaccharide conjugate.

The present invention relates to an immunogenic composition comprising a glycoconjugate from S. pneumoniae serotype 15B or 15C and a glycoconjugate from S. pneumoniae serotype 15A for use in a method of immunizing a human subject against infection by S. pneumoniae serotype 15A, 15B and 15C. Preferably said composition does not comprise capsular saccharide from S. pneumoniae serotypes 15C when a glycoconjugate from S. pneumoniae serotype 15B is present in the composition and does not comprise capsular saccharide from S. pneumoniae serotypes 15B when a glycoconjugate from S. pneumoniae serotype 15C is present.

The present invention relates to an immunogenic composition comprising a glycoconjugate from S. pneumoniae serotype 15B and a glycoconjugate from S. pneumoniae serotype 15A for use in a method of immunizing a human subject against infection by S. pneumoniae serotype 15A, 15B and 15C. Preferably said composition does not comprise capsular saccharide from S. pneumoniae serotypes 15C.

The present invention also relates to an immunogenic composition comprising a glycoconjugate from S. pneumoniae serotype 15C and a glycoconjugate from S. pneumoniae serotype 15A for use in a method of immunizing a human subject against infection by S. pneumoniae serotype 15A, 15B and 15C. Preferably said composition does not comprise capsular saccharide from S. pneumoniae serotypes 15B.

The present invention also relates to an immunogenic composition comprising a glycoconjugate from S. pneumoniae serotype 15B, a glycoconjugate from S. pneumoniae serotype 15B and a glycoconjugate from S. pneumoniae serotype 15A for use in a method of immunizing a human subject against infection by S. pneumoniae serotype 15A, 15B and 15C.

In an aspect the invention relates to a set of immunogenic compositions comprising: (a) a first immunogenic composition comprising a glycoconjugate from S. pneumoniae serotype 15C; and (b) a second immunogenic composition comprising a glycoconjugate from S. pneumoniae serotype 15A, wherein said compositions do not comprise capsular saccharide from S. pneumoniae serotypes 15B, for use in a method of immunizing a human subject against infection by S. pneumoniae serotype 15A, 15B and 15C, and wherein said compositions are for simultaneous, concurrent, concomitant or sequential administration.

In an aspect the invention relates to a set of immunogenic compositions comprising: (a) a first immunogenic composition comprising a glycoconjugate from S. pneumoniae serotype 15B; and (b) a second immunogenic composition comprising a glycoconjugate from S. pneumoniae serotype 15A, wherein said compositions do not comprise capsular saccharide from S. pneumoniae serotypes 15C, for use in a method of immunizing a human subject against infection by S. pneumoniae serotype 15A, 15B and 15C, and wherein said compositions are for simultaneous, concurrent, concomitant or sequential administration.

In an aspect the invention relates to a set of immunogenic compositions comprising: (a) a first immunogenic composition comprising a glycoconjugate from S. pneumoniae serotype 15B; and (b) a second immunogenic composition comprising a glycoconjugate from S. pneumoniae serotype 15A and a glycoconjugate from S. pneumoniae serotype 15C, for use in a method of immunizing a human subject against infection by S. pneumoniae serotype 15A, 15B and 15C, and wherein said compositions are for simultaneous, concurrent, concomitant or sequential administration.

In an aspect the invention relates to a method of immunizing a human subject against infection by S. pneumoniae serotype 15A, 15B and 15C, said method comprising administering to the human subject an immunologically effective amount of an immunogenic composition comprising a glycoconjugate from S. pneumoniae serotype 15B or 15C and a glycoconjugate from S. pneumoniae serotype 15A, wherein said composition does not comprise capsular saccharide from S. pneumoniae serotypes 15C when a glycoconjugate from S. pneumoniae serotype 15B is present in the composition and does not comprise capsular saccharide from S. pneumoniae serotypes 15B when a glycoconjugate from S. pneumoniae serotype 15C is present

In an aspect the invention relates to a method of immunizing a human subject against infection by S. pneumoniae serotype 15A, 15B and 15C, said method comprising administering to the human subject an immunologically effective amount of an immunogenic composition comprising a glycoconjugate from S. pneumoniae serotype 15B and a glycoconjugate from S. pneumoniae serotype 15A, wherein said composition does not comprise capsular saccharide from S. pneumoniae serotype 15C.

In an aspect the invention relates to a method of immunizing a human subject against infection by S. pneumoniae serotype 15A, 15B and 15C, said method comprising administering to the human subject an immunologically effective amount of an immunogenic composition comprising a glycoconjugate from S. pneumoniae serotype 15C and a glycoconjugate from S. pneumoniae serotype 15A, wherein said composition does not comprise capsular saccharide from S. pneumoniae serotype 15B.

In an aspect the invention relates to a method of immunizing a human subject against infection by S. pneumoniae serotype 15A, 15B and 15C, said method comprising administering to the human subject an immunologically effective amount of an immunogenic composition comprising a glycoconjugate from S. pneumoniae serotype 15C, a glycoconjugate from S. pneumoniae serotype 15C and a glycoconjugate from S. pneumoniae serotype 15A.

The present invention relates to an immunogenic composition comprising a glycoconjugate from S. pneumoniae serotype 15B or 15C and a glycoconjugate from S. pneumoniae serotype 15A for use in a method of preventing, treating or ameliorating an infection, disease or condition associated with infection by S. pneumoniae serotypes 15A, 15B and 15C in a human subject. Preferably said composition does not comprise capsular saccharide from S. pneumoniae serotype 15C when a glycoconjugate from S. pneumoniae serotype 15B is present in the composition and does not comprise capsular saccharide from S. pneumoniae serotypes 15B when a glycoconjugate from S. pneumoniae serotype 15C is present.

The present invention relates to an immunogenic composition comprising a glycoconjugate from S. pneumoniae serotype 15B and a glycoconjugate from S. pneumoniae serotype 15A for use in a method of preventing, treating or ameliorating an infection, disease or condition associated with infection by S. pneumoniae serotypes 15A, 15B and 15C in a human subject. Preferably said composition does not comprise capsular saccharide from S. pneumoniae serotypes 15C.

The present invention also relates to an immunogenic composition comprising a glycoconjugate from S. pneumoniae serotype 15C and a glycoconjugate from S. pneumoniae serotype 15A for use in a method of preventing, treating or ameliorating an infection, disease or condition associated with infection by S. pneumoniae serotypes 15A, 15B and 15C in a human subject. Preferably said composition does not comprise capsular saccharide from S. pneumoniae serotypes 15B.

The present invention also relates to an immunogenic composition comprising a glycoconjugate from S. pneumoniae serotype 15B, a glycoconjugate from S. pneumoniae serotype 15C and a glycoconjugate from S. pneumoniae serotype 15A for use in a method of preventing, treating or ameliorating an infection, disease or condition associated with infection by S. pneumoniae serotypes 15A, 15B and 15C in a human subject.

In an aspect the invention relates to a set of immunogenic compositions comprising: (a) a first immunogenic composition comprising a glycoconjugate from S. pneumoniae serotype 15C; and (b) a second immunogenic composition comprising a glycoconjugate from S. pneumoniae serotype 15A, wherein said compositions do not comprise capsular saccharide from S. pneumoniae serotypes 15B, for use in a method of preventing, treating or ameliorating an infection, disease or condition associated with infection by S. pneumoniae serotypes 15A, 15B and 15C in a human subject, and wherein said compositions are for simultaneous, concurrent, concomitant or sequential administration.

In an aspect the invention relates to a set of immunogenic compositions comprising: (a) a first immunogenic composition comprising a glycoconjugate from S. pneumoniae serotype 15B; and (b) a second immunogenic composition comprising a glycoconjugate from S. pneumoniae serotype 15A, wherein said compositions do not comprise capsular saccharide from S. pneumoniae serotypes 15C, for use in a method of preventing, treating or ameliorating an infection, disease or condition associated with infection by S. pneumoniae serotypes 15A, 15B and 15C in a human subject, and wherein said compositions are for simultaneous, concurrent, concomitant or sequential administration.

In an aspect the invention relates to a set of immunogenic compositions comprising: (a) a first immunogenic composition comprising a glycoconjugate from S. pneumoniae serotype 15B; and (b) a second immunogenic composition comprising a glycoconjugate from S. pneumoniae serotype 15C and a glycoconjugate from S. pneumoniae serotype 15A, for use in a method of preventing, treating or ameliorating an infection, disease or condition associated with infection by S. pneumoniae serotypes 15A, 15B and 15C in a human subject, and wherein said compositions are for simultaneous, concurrent, concomitant or sequential administration.

In an aspect the invention relates to a method of preventing, treating or ameliorating an infection, disease or condition associated with infection by S. pneumoniae serotypes 15A, 15B and 15C, said method comprising administering to the human subject an immunologically effective amount of an immunogenic composition comprising a glycoconjugate from S. pneumoniae serotype 15B or 15C and a glycoconjugate from S. pneumoniae serotype 15A, wherein said composition does not comprise capsular saccharide from S. pneumoniae serotypes 15C when a glycoconjugate from S. pneumoniae serotype 15B is present in the composition and does not comprise capsular saccharide from S. pneumoniae serotypes 15B when a glycoconjugate from S. pneumoniae serotype 15C is present

In an aspect the invention relates to a method of preventing, treating or ameliorating an infection, disease or condition associated with infection by S. pneumoniae serotypes 15A, 15B and 15C, said method comprising administering to the human subject an immunologically effective amount of an immunogenic composition comprising a glycoconjugate from S. pneumoniae serotype 15B and a glycoconjugate from S. pneumoniae serotype 15A, wherein said composition does not comprise capsular saccharide from S. pneumoniae serotype 15C.

In an aspect the invention relates to a method of preventing, treating or ameliorating an infection, disease or condition associated with infection by S. pneumoniae serotypes 15A, 15B and 15C, said method comprising administering to the human subject an immunologically effective amount of an immunogenic composition comprising a glycoconjugate from S. pneumoniae serotype 15C and a glycoconjugate from S. pneumoniae serotype 15A, wherein said composition does not comprise capsular saccharide from S. pneumoniae serotype 15B.

In an aspect the invention relates to a method of preventing, treating or ameliorating an infection, disease or condition associated with infection by S. pneumoniae serotypes 15A, 15B and 15C, said method comprising administering to the human subject an immunologically effective amount of an immunogenic composition comprising a glycoconjugate from S. pneumoniae serotype 15B, a glycoconjugate from S. pneumoniae serotype 15C and a glycoconjugate from S. pneumoniae serotype 15A.

In one aspect, the above immunogenic compositions or set of immunogenic compositions further comprise at least one glycoconjugate from S. pneumoniae serotypes 4, 6B, 9V, 14, 19F and/or 23F.

In an aspect the above immunogenic compositions or set of immunogenic compositions further comprise at least one glycoconjugate from S. pneumoniae serotypes 4, 6B, 9V, 14, 18C, 19F and/or 23F In an aspect the above immunogenic compositions or set of immunogenic compositions further comprise at least one glycoconjugate from S. pneumoniae serotype 1, 5 and/or 7F.

In an aspect the above immunogenic compositions or set of immunogenic compositions further comprise at least one glycoconjugate from S. pneumoniae serotype 6A and/or 19A.

In an aspect the above immunogenic compositions or set of immunogenic compositions further comprise at least one glycoconjugate from S. pneumoniae serotype 3, 15B, 22F, 33F, 12F, 10A, 11A and/or 8.

In a further aspect the above immunogenic compositions or set of immunogenic compositions further comprise at least one glycoconjugate from S. pneumoniae serotype 2, 15C, 17F and/or 20.

In an aspect the above immunogenic compositions or set of immunogenic compositions further comprise at least one glycoconjugate from S. pneumoniae serotype 9N.

In a further aspect the immunogenic compositions or set of immunogenic compositions are a 7, 8, 9, 10, 11, 12, 13, 14, 15, 16, 17, 18, 19, 20, 21, 22, 23, 24 or 25-valent pneumococcal conjugate composition.

In a further aspect the glycoconjugates of the immunogenic compositions or set of immunogenic compositions are individually conjugated to CRM₁₉₇.

In one aspect, if present the glycoconjugates from S. pneumoniae serotypes 1, 4, 5, 6B, 7F, 9V, 14 and/or 23F of the immunogenic compositions are individually conjugated to PD, and if present, the glycoconjugate from S. pneumoniae serotype 18C is conjugated to TT and if present the glycoconjugate from S. pneumoniae serotype 19F is conjugated to DT.

In one aspect, the glycoconjugates are prepared using reductive amination chemistry. In one aspect, the glycoconjugates are prepared using CDAP chemistry.

The immunogenic compositions or set of immunogenic compositions may further comprise antigens from other pathogens, and/or at least one adjuvant such as aluminum phosphate, aluminum sulphate or aluminum hydroxide.

In an aspect the immunogenic compositions or set of immunogenic compositions are able to elicit IgG antibodies in human which are capable of binding S. pneumoniae serotypes 15A, 15B and 15C polysaccharide at a concentration of at least 0.35 μg/ml as determined by ELISA assay.

In an aspect the immunogenic compositions or set of immunogenic compositions are able to elicit a titer of at least 1:8 against S. pneumoniae serotype 15A, 15B and 15C in at least 50% of the subjects as determined by in vitro opsonophagocytic killing assay (OPA).

In an aspect the immunogenic compositions or set of immunogenic compositions are able to significantly increase the proportion of responders against S. pneumoniae serotypes 15A, 15B and 15C as compared to the pre-immunized population.

In an aspect the immunogenic compositions or set of immunogenic compositions are able to significantly increase the OPA titers of human subjects against S. pneumoniae serotype 15A, 15B and 15C as compared to the pre-immunized population.

In one aspect the present invention relates to the use of the immunogenic composition disclosed in the present document for the manufacture of a medicament for preventing, treating or ameliorating an infection, disease or condition caused by S. pneumoniae serotypes 15A, 15B and 15C in a subject, for use to prevent to prevent serotypes 15A, 15B and 15C S. pneumoniae infection in a subject or for use in a method to protect or treat a human susceptible to S. pneumoniae serotypes 15A, 15B and 15C infection, by means of administering said immunogenic compositions via a systemic or mucosal route.

The invention further relates to a kit comprising an immunogenic composition or a set of immunogenic compositions disclosed herein and an information leaflet, wherein said information leaflet mentions the ability of the composition or of the set of immunogenic compositions to elicit functional antibodies against S. pneumoniae serotypes 15A, 15B and 15C.

### FIGURES

FIG. 1 Reverse cumulative distribution curves of OPA titers to serotype 15C from serum panel from vaccinees administered 20vPnC, which contains 15B capsular polysaccharide conjugate, or 13vPnC, which does not contain 15B capsular polysaccharide conjugate (see example 3) (Study B7471002 (ClinicalTrials.gov Identifier: NCT03313037)). The plots represent the percent of sera with OPA positive titer (i.e., ≥1:8).

1 Immunogenic Compositions Comprising a Glycoconjugate from S. pneumoniae Serotype 15B and/or 15C and a Glycoconjugate from S. pneumoniae Serotype 15A of the Invention

Immunogenic compositions of the present invention will typically comprise conjugated capsular saccharide antigens (also named glycoconjugates), wherein the saccharides are derived from serotypes of S. pneumoniae.

Preferably, the number of S. pneumoniae capsular saccharides can range from 2 serotypes (or “v”, valences) to 25 different serotypes (25v). In one embodiment there are 2 different serotypes. In one embodiment there are 3 different serotypes. In one embodiment there are 4 different serotypes. In one embodiment there are 5 different serotypes. In one embodiment there are 6 different serotypes. In one embodiment there are 7 different serotypes. In one embodiment there are 8 different serotypes. In one embodiment there are 9 different serotypes. In one embodiment there are 10 different serotypes. In one embodiment there are 11 different serotypes. In one embodiment there are 12 different serotypes. In one embodiment there are 13 different serotypes. In one embodiment there are 14 different serotypes. In one embodiment there are 15 different serotypes. In one embodiment there are 16 different serotypes. In an embodiment there are 17 different serotypes. In an embodiment there are 18 different serotypes. In an embodiment there are 19 different serotypes. In an embodiment there are 20 different serotypes. In an embodiment there are 21 different serotypes. In an embodiment there are 22 different serotypes. In an embodiment there are 23 different serotypes. In an embodiment there are 24 different serotypes. In an embodiment there are 25 different serotypes. The capsular saccharides are conjugated to a carrier protein to form glycoconjugates as described here below.

If the protein carrier is the same for 2 or more saccharides in the composition, the saccharides could be conjugated to the same molecule of the protein carrier (carrier molecules having 2 or more different saccharides conjugated to it) [see for instance WO 2004/083251].

In a preferred embodiment though, the saccharides are each individually conjugated to different molecules of the protein carrier (each molecule of protein carrier only having one type of saccharide conjugated to it). In said embodiment, the capsular saccharides are said to be individually conjugated to the carrier protein.

For the purposes of the invention the term ‘glycoconjugate’ indicates a capsular saccharide linked covalently to a carrier protein. In one embodiment a capsular saccharide is linked directly to a carrier protein. In a second embodiment a bacterial saccharide is linked to a protein through a spacer/linker.

1.1 Capsular Saccharide of the Invention

The term “saccharide” throughout this specification may indicate polysaccharide or oligosaccharide and includes both. In frequent embodiments, the saccharide is a polysaccharide, in particular a S. pneumoniae capsular polysaccharide.

Capsular polysaccharides are prepared by standard techniques known to those of ordinary skill in the art.

Typically, capsular polysaccharides are produced by growing each S. pneumoniae serotype in a medium (e.g., in a soy-based medium), the polysaccharides are then prepared from the bacteria culture. Bacterial strains of S. pneumoniae used to make the respective polysaccharides that are used in the glycoconjugates of the invention may be obtained from established culture collections or clinical specimens.

The population of the organism (each S. pneumoniae serotype) is often scaled up from a seed vial to seed bottles and passaged through one or more seed fermentors of increasing volume until production scale fermentation volumes are reached. At the end of the growth cycle the cells are lysed and the lysate broth is then harvested for downstream (purification) processing (see for example WO 2006/110381, WO 2008/118752, and U.S. Patent App. Pub. Nos. 2006/0228380, 2006/0228381, 2008/0102498 and 2008/0286838).

The individual polysaccharides are typically purified through centrifugation, precipitation, ultra-filtration, and/or column chromatography (see for example WO 2006/110352 and WO 2008/118752).

Purified polysaccharides may be activated (e.g., chemically activated) to make them capable of reacting (e.g., either directly to the carrier protein of via a linker such as an eTEC spacer) and then incorporated into glycoconjugates of the invention, as further described herein.

S. pneumoniae capsular polysaccharides comprise repeating oligosaccharide units which may contain up to 8 sugar residues.

In an embodiment, capsular saccharide of the invention may be one oligosaccharide unit, or a shorter than native length saccharide chain of repeating oligosaccharide units. In an embodiment, capsular saccharide of the invention is one repeating oligosaccharide unit of the relevant serotype.

In an embodiment, capsular saccharide of the invention may be oligosaccharides. Oligosaccharides have a low number of repeat units (typically 5-15 repeat units) and are typically derived synthetically or by hydrolysis of polysaccharides.

In an embodiment, all of the capsular saccharides of the present invention and in the immunogenic compositions of the present invention are polysaccharides. High molecular weight capsular polysaccharides are able to induce certain antibody immune responses due to the epitopes present on the antigenic surface. The isolation and purification of high molecular weight capsular polysaccharides is preferably contemplated for use in the conjugates, compositions and methods of the present invention.

In some embodiments, the purified polysaccharides before conjugation have a molecular weight of between 5 kDa and 4,000 kDa. In other such embodiments, the polysaccharide has a molecular weight of between 10 kDa and 4,000 kDa; between 50 kDa and 4,000 kDa; between 50 kDa and 3,000 kDa; between 50 kDa and 2,000 kDa; between 50 kDa and 1,500 kDa; between 50 kDa and 1,000 kDa; between 50 kDa and 750 kDa; between 50 kDa and 500 kDa; between 100 kDa and 4,000 kDa; between 100 kDa and 3,000 kDa; 100 kDa and 2,000 kDa; between 100 kDa and 1,500 kDa; between 100 kDa and 1,000 kDa; between 100 kDa and 750 kDa; between 100 kDa and 500 kDa; between 100 and 400 kDa; between 200 kDa and 4,000 kDa; between 200 kDa and 3,000 kDa; between 200 kDa and 2,000 kDa; between 200 kDa and 1,500 kDa; between 200 kDa and 1,000 kDa. In an embodiment, the capsular polysaccharide has a molecular weight of between or between 200 kDa and 500 kDa. In another embodiment, the capsular polysaccharide has a molecular weight of between 100 kDa to 500 kDa.

In further embodiments, the capsular polysaccharide has a molecular weight of between 5 kDa to 100 kDa; 7 kDa to 100 kDa; 10 kDa to 100 kDa; 20 kDa to 100 kDa; 30 kDa to 100 kDa; 40 kDa to 100 kDa; 50 kDa to 100 kDa; 60 kDa to 100 kDa; 70 kDa to 100 kDa; 80 kDa to 100 kDa; 90 kDa to 100 kDa; 5 kDa to 90 KDa; 5 kDa to 80 kDa; 5 kDa to 70 kDa; 5 kDa to 60 kDa; 5 kDa to 50 kDa; 5 kDa to 40 kDa; 5 kDa to 30 kDa; 5 kDa to 20 kDa or 5 kDa to 10 kDa. Any whole number integer within any of the above ranges is contemplated as an embodiment of the disclosure.

A polysaccharide can become slightly reduced in size during normal purification procedures. Additionally, polysaccharide can be subjected to sizing techniques before conjugation. Mechanical or chemical sizing maybe employed.

For example, saccharides of serotype 15B can be mechanically sized (see e.g. WO2015/110942). Saccharide of serotype 15A can be mechanically sized (see e.g. WO2019/139692, examples 15-16). Saccharide of serotype 15C can be mechanically sized (see e.g. WO2019/139692, examples 18-19).

Saccharide of serotype 15A can also be chemically sized. Saccharide of serotype 15C can also be chemically sized. Chemical hydrolysis maybe conducted using acetic acid.

Saccharide of serotype 15B is preferably not chemically sized in order to preserve an 0-acetyl content close to content found in the natural capsular polysaccharide.

Mechanical sizing maybe conducted using High Pressure Homogenization Shearing. The molecular weight ranges mentioned above refer to purified polysaccharides before conjugation (e.g., before activation).

In a preferred embodiment the purified polysaccharides, are capsular polysaccharide from serotypes 1, 2, 3, 4, 5, 6A, 6B, 7F, 8, 9V, 9N, 10A, 11A, 12F, 14, 15A, 15B, 15C, 17F, 18C, 19A, 19F, 20, 22F, 23F or 33F of S. pneumoniae, wherein the capsular polysaccharide has a molecular weight falling within one of the molecular weight ranges as described here above.

In another preferred embodiment the purified polysaccharides, are capsular polysaccharide from serotypes 1, 2, 3, 4, 5, 6A, 6B, 7F, 8, 9V, 9N, 10A, 11A, 12F, 14, 15A, 15B, 15C, 17F, 18C, 19A, 19F, 20, 22F, 23B, 23F, 24F, 33F or 35B of S. pneumoniae, wherein the capsular polysaccharide has a molecular weight falling within one of the molecular weight ranges as described here above.

As used herein, the term “molecular weight” of polysaccharide or of carrier protein-polysaccharide conjugate refers to the weight average molecular weight (Mw) which can be measured by size exclusion chromatography (SEC) combined with multiangle laser light scattering detector (MALLS).

In some embodiments, the pneumococcal saccharides from serotypes 9V, 18C, 11A, 15B, 22F and/or 33F of the invention are O-acetylated. In some embodiments, the pneumococcal saccharides from serotypes 9V, 11A, 15B, 22F and/or 33F of the invention are O-acetylated. In a preferred embodiment, the pneumococcal saccharide from serotype 18C of the invention is de-O-acetylated. For example, saccharides of serotype 18C can be de-O-acetylated by acidic treatment (see e.g. WO2006/110381, page 37 lines 1-4).

The degree of O-acetylation of the polysaccharide can be determined by any method known in the art, for example, by proton NMR (see for example Lemercinier et al. (1996) Carbohydrate Research 296:83-96, Jones et al. (2002) J. Pharmaceutical and Biomedical Analysis 30:1233-1247, WO 2005/033148 and WO 00/56357). Another commonly used method is described in Hestrin (1949) J. Biol. Chem. 180:249-261. Preferably, the presence of O-acetyl groups is determined by ion-HPLC analysis.

The purified polysaccharides described herein are chemically activated to make the saccharides capable of reacting with the carrier protein. These pneumococcal conjugates are prepared by separate processes and formulated into a single dosage formulation as described below.

1.2 Glycoconjugates of the Invention

The purified saccharides are chemically activated to make the saccharides capable of reacting with the carrier protein (i.e., activated saccharides), either directly or via a linker. Once activated, each capsular saccharide is separately conjugated to a carrier protein to form a glycoconjugate. In one embodiment, each capsular saccharide is conjugated to the same carrier protein. The chemical activation of the saccharides and subsequent conjugation to the carrier protein can be achieved by the activation and conjugation methods disclosed herein.

Capsular polysaccharides from S. pneumoniae are prepared as disclosed above.

In an embodiment, the polysaccharides are activated with 1-cyano-4-dimethylamino pyridinium tetrafluoroborate (CDAP) to form a cyanate ester. The activated polysaccharide is then coupled directly or via a spacer (linker) group to an amino group on the carrier protein (preferably CRM₁₉₇). For example, the spacer could be cystamine or cysteamine to give a thiolated polysaccharide which could be coupled to the carrier via a thioether linkage obtained after reaction with a maleimide-activated carrier protein (for example using N-[γ-maleimidobutyrloxy]succinimide ester (GMBS)) or a haloacetylated carrier protein (for example using iodoacetimide, N-succinimidyl bromoacetate (SBA; SIB), N-succinimidyl(4-iodoacetyl)aminobenzoate (SIAB), sulfosuccinimidyl(4-iodoacetyl)aminobenzoate (sulfo-SIAB), N-succinimidyl iodoacetate (SIA), or succinimidyl 3-[bromoacetamido]proprionate (SBAP)). Preferably, the cyanate ester (optionally made by CDAP chemistry) is coupled with hexane diamine or adipic acid dihydrazide (ADH) and the amino-derivatised saccharide is conjugated to the carrier protein (e.g., CRM₁₉₇) using carbodiimide (e.g., EDAC or EDC) chemistry via a carboxyl group on the protein carrier. Such conjugates are described for example in WO 93/15760, WO 95/08348 and WO 96/129094.

In an embodiment of the present invention, the glycoconjugates from S. pneumoniae serotypes 1, 2, 3, 4, 5, 6A, 6B, 7F, 8, 9V, 9N, 10A, 11A, 12F, 14, 15B, 15C, 17F, 18C, 19A, 19F, 20, 22F, 23F and/or 33F are prepared using CDAP chemistry. In an embodiment of the present invention, the glycoconjugates from S. pneumoniae serotypes 1, 4, 5, 6B, 7F, 9V, 14, 18C, 19F, and 23F are prepared using CDAP chemistry. In an embodiment of the present invention, the glycoconjugates from S. pneumoniae serotypes 1, 4, 5, 6A, 6B, 7F, 9V, 14, 18C, 19F, and 23F are prepared using CDAP chemistry. In an embodiment of the present invention, the glycoconjugates from S. pneumoniae serotypes 1, 4, 5, 6B, 7F, 9V, 14, 18C, 19A, 19F, and 23F are prepared using CDAP chemistry. In an embodiment of the present invention, the glycoconjugates from S. pneumoniae serotypes 1, 4, 5, 6A, 6B, 7F, 9V, 14, 18C, 19A, 19F, and 23F are prepared using CDAP chemistry. In an embodiment of the present invention, the glycoconjugates from S. pneumoniae serotypes 1, 3, 4, 5, 6A, 6B, 7F, 9V, 14, 18C, 19A, 19F, and 23F are prepared using CDAP chemistry.

Other suitable techniques for conjugation use carbodiimides, hydrazides, active esters, norborane, p-nitrobenzoic acid, N-hydroxysuccinimide, S—NHS, EDC, TSTU. Many are described in International Patent Application Publication No. WO 98/42721. Conjugation may involve a carbonyl linker which may be formed by reaction of a free hydroxyl group of the saccharide with CDI (see Bethell et al. (1979) 1. Biol. Chern. 254:2572-2574; Hearn et al. (1981) J. Chromatogr. 218:509-518) followed by reaction with a protein to form a carbamate linkage. This may involve reduction of the anomeric terminus to a primary hydroxyl group, optional protection/deprotection of the primary hydroxyl group, reaction of the primary hydroxyl group with CDI to form a CDI carbamate intermediate and coupling the CDI carbamate intermediate with an amino group on a protein.

In a preferred embodiment, at least one of capsular polysaccharides from serotypes 1, 2, 3, 4, 5, 6A, 6B, 7F, 8, 9V, 9N, 10A, 11A, 12F, 14, 15A, 15B, 15C, 17F, 18C, 19A, 19F, 20, 22F, 23F and 33F of S. pneumoniae is conjugated to the carrier protein by reductive amination (such as described in U.S. Patent Appl. Pub. Nos. 2006/0228380, 2007/184072, 2007/0231340 and 2007/0184071, WO 2006/110381, WO 2008/079653, and WO 2008/143709).

In another preferred embodiment, at least one of capsular polysaccharides from serotypes 1, 2, 3, 4, 5, 6A, 6B, 7F, 8, 9V, 9N, 10A, 11A, 12F, 14, 15A, 15B, 15C, 17F, 18C, 19A, 19F, 20, 22F, 23B, 23F, 24F, 33F and 35B of S. pneumoniae is conjugated to the carrier protein by reductive amination.

In a preferred embodiment of the present invention, the glycoconjugate from S. pneumoniae serotype 15A is prepared by reductive amination. In a preferred embodiment of the present invention, the glycoconjugate from S. pneumoniae serotype 15B is prepared by reductive amination. In a preferred embodiment of the present invention, the glycoconjugate from S. pneumoniae serotype 15C is prepared by reductive amination.

In a preferred embodiment of the present invention, the glycoconjugate from S. pneumoniae serotype 18C is prepared by reductive amination. In a preferred embodiment of the present invention, the glycoconjugate from S. pneumoniae serotype 6A is prepared by reductive amination. In a preferred embodiment of the present invention, the glycoconjugate from S. pneumoniae serotype 19A is prepared by reductive amination. In a preferred embodiment of the present invention, the glycoconjugate from S. pneumoniae serotype 3 is prepared by reductive amination. In a preferred embodiment of the present invention, the glycoconjugates from S. pneumoniae serotypes 6A and 19A are prepared by reductive amination. In a preferred embodiment of the present invention, the glycoconjugates from S. pneumoniae serotypes 3, 6A and 19A are prepared by reductive amination.

In an embodiment of the present invention, the glycoconjugates from S. pneumoniae serotypes 4, 6B, 9V, 14, 18C, 19F and 23F are prepared by reductive amination. In an embodiment of the present invention, the glycoconjugates from S. pneumoniae serotypes 1, 4, 6B, 9V, 14, 18C, 19F and 23F are prepared by reductive amination. In a preferred embodiment of the present invention, the glycoconjugates from S. pneumoniae serotypes 1, 4, 5, 6B, 9V, 14, 18C, 19F and 23F are prepared by reductive amination. In an embodiment of the present invention, the glycoconjugates from S. pneumoniae serotypes 1, 4, 5, 6B, 7F, 9V, 14, 18C, 19F and 23F are prepared by reductive amination. In an embodiment of the present invention, the glycoconjugates from S. pneumoniae serotypes 1, 4, 5, 6A, 6B, 7F, 9V, 14, 18C, 19F and 23F are prepared by reductive amination. In an embodiment of the present invention, the glycoconjugates from S. pneumoniae serotypes 1, 4, 5, 6A, 6B, 7F, 9V, 14, 18C, 19A, 19F and 23F are prepared by reductive amination.

In a preferred embodiment of the present invention, the glycoconjugates from S. pneumoniae serotypes 1, 3, 4, 5, 6A, 6B, 7F, 9V, 14, 18C, 19A, 19F and 23F are all prepared by reductive amination.

In another preferred embodiment, the glycoconjugates from S. pneumoniae serotypes 1, 3, 4, 5, 6A, 6B, 7F, 9V, 14, 18C, 19A, 19F, 22F and 23F are all prepared by reductive amination.

In another preferred embodiment, the glycoconjugates from S. pneumoniae serotypes 1, 3, 4, 5, 6A, 6B, 7F, 9V, 14, 15A, 15B, 18C, 19A, 19F, 22F and 23F are all prepared by reductive amination.

In another preferred embodiment, the glycoconjugates from S. pneumoniae serotypes 1, 3, 4, 5, 6A, 6B, 7F, 8, 9V, 10A, 11A, 12F, 14, 15A, 15B, 18C, 19A, 19F, 22F and 23F are all prepared by reductive amination.

In another preferred embodiment, the glycoconjugates from S. pneumoniae serotypes 1, 2, 3, 4, 5, 6A, 6B, 7F, 9V, 14, 18C, 19A, 19F and 23F are all prepared by reductive amination.

In another embodiment, the glycoconjugates from S. pneumoniae serotypes 1, 4, 5, 6A, 6B, 7F, 9V, 12F, 14, 15A, 15C, 18C, 19A, 19F, 22F, 23F and 33F are all prepared by reductive amination.

In another preferred embodiment, the glycoconjugates from S. pneumoniae serotypes 1, 3, 4, 5, 6A, 6B, 7F, 8, 9V, 10A, 11A, 14, 15A, 15B, 18C, 19A, 19F, 22F and 23F are all prepared by reductive amination.

In another preferred embodiment, the glycoconjugates from S. pneumoniae serotypes 1, 3, 4, 5, 6A, 6B, 7F, 8, 9V, 10A, 11A, 12F, 14, 15A, 15B, 18C, 19A, 19F, 22F and 23F are all prepared by reductive amination.

In another embodiment, the glycoconjugates from S. pneumoniae serotypes 1, 3, 4, 5, 6A, 6B, 7F, 8, 9V, 10A, 11A, 12F, 14, 15A, 15B, 18C, 19A, 19F, 22F, 23F and 33F are all prepared by reductive amination.

In another embodiment, the glycoconjugates from S. pneumoniae serotypes 1, 2, 3, 4, 5, 6A, 6B, 7F, 8, 9V, 10A, 11A, 12F, 14, 15A, 15B, 17F, 18C, 19A, 19F, 20, 22F, 23F and 33F are all prepared by reductive amination.

In another embodiment, the glycoconjugates from S. pneumoniae serotypes 1, 2, 3, 4, 5, 6A, 6B, 7F, 8, 9V, 9N, 10A, 11A, 12F, 14, 15A, 15B, 17F, 18C, 19A, 19F, 20, 22F, 23F and 33F are all prepared by reductive amination.

In another embodiment, the glycoconjugates from S. pneumoniae serotypes 1, 2, 3, 4, 5, 6A, 6B, 7F, 8, 9V, 9N, 10A, 11A, 12F, 14, 15A, 15B, 15C, 17F, 18C, 19A, 19F, 20, 22F, 23B, 23F, 24F, 33F and 35B are all prepared by reductive amination.

Reductive amination involves two steps, (1) oxidation of the polysaccharide, (2) reduction of the activated polysaccharide and a carrier protein to form a conjugate. Before oxidation, the polysaccharide is optionally hydrolyzed. Mechanical or chemical hydrolysis maybe employed. Chemical hydrolysis maybe conducted using acetic acid.

The oxidation step may involve reaction with periodate. For the purpose of the present invention, the term “periodate” includes both periodate and periodic acid; the term also includes both metaperiodate (IO₄⁻) and orthoperiodate (IO₆⁵⁻) and includes the various salts of periodate (e.g., sodium periodate and potassium periodate). In an embodiment the capsular polysaccharide is oxidized in the presence of metaperiodate, preferably in the presence of sodium periodate (NalO4). In another embodiment the capsular polysaccharide is oxydized in the presence of orthoperiodate, preferably in the presence of periodic acid.

In an embodiment, the oxidizing agent is a stable nitroxyl or nitroxide radical compound, such as piperidine-N-oxy or pyrrolidine-N-oxy compounds, in the presence of an oxidant to selectively oxidize primary hydroxyls (as described in WO 2014/097099). In said reaction, the actual oxidant is the N-oxoammonium salt, in a catalytic cycle. In an aspect, said stable nitroxyl or nitroxide radical compound are piperidine-N-oxy or pyrrolidine-N-oxy compounds. In an aspect, said stable nitroxyl or nitroxide radical compound bears a TEMPO (2,2,6,6-tetramethyl-1-piperidinyloxy) or a PROXYL (2,2,5,5-tetramethyl-1-pyrrolidinyloxy) moiety. In an aspect, said stable nitroxyl radical compound is TEMPO or a derivative thereof. In an aspect, said oxidant is a molecule bearing a N-halo moiety. In an aspect, said oxidant is N-ChloroSuccinimide, N-Bromosuccinimide, N-lodosuccinimide, Dichloroisocyanuric acid, 1,3,5-trichloro-1,3,5-triazinane-2,4,6-trione, Dibromoisocyanuric acid, 1,3,5-tribromo-1,3,5-triazinane-2,4,6-trione, Diiodoisocyanuric acid or 1,3,5-triiodo-1,3,5-triazinane-2,4,6-trione. In an aspect, said oxidant is a molecule bearing a N-halo moiety. In an aspect, said oxidant is selected from the group consisting of N-ChloroSuccinimide, N-Bromosuccinimide, N-lodosuccinimide, Dichloroisocyanuric acid, 1,3,5-trichloro-1,3,5-triazinane-2,4,6-trione, Dibromoisocyanuric acid, 1,3,5-tribromo-1,3,5-triazinane-2,4,6-trione, Diiodoisocyanuric acid and 1,3,5-triiodo-1,3,5-triazinane-2,4,6-trione. Preferably said oxidant is N-Chlorosuccinimide.

In a preferred embodiment, capsular polysaccharides from serotypes 12F S. pneumoniae are conjugated to the carrier protein by reductive amination, wherein the oxidizing agent is 2,2,6,6-Tetramethyl-1-piperidinyloxy (TEMPO) free radical and N-Chlorosuccinimide (NCS) as the cooxidant (as described in WO 2014/097099). Therefore in one aspect, the glycoconjugates from S. pneumoniae serotype 12F are obtainable by a method comprising the steps of: a) reacting a 12F saccharide with 2,2,6,6-tetramethyl-1-piperidinyloxy (TEMPO) and N-chlorosuccinimide (NCS) in an aqueous solvent to produce an activated saccharide; and b) reacting the activated saccharide with a carrier protein comprising one or more amine groups (said method is designated “TEMPO/NCS-reductive amination” thereafter).

Optionally the oxidation reaction is quenched by addition of a quenching agent. The quenching agent maybe selected from vicinal diols, 1,2-aminoalcohols, amino acids, glutathione, sulfite, bisulfate, dithionite, metabisulfite, thiosulfate, phosphites, hypophosphites or phosphorous acid (such as glycerol, ethylene glycol, propan-1,2-diol, butan-1,2-diol or butan-2,3-diol, ascorbic acid).

Following the oxidation step of the polysaccharide, the polysaccharide is said to be activated and is referred to an “activated polysaccharide” here below. The activated polysaccharide and the carrier protein may be lyophilised (freeze-dried), either independently (discrete lyophilization) or together (co-lyophilized). In one embodiment the activated polysaccharide and the carrier protein are co-lyophilized. In another embodiment the activated polysaccharide and the carrier protein are lyophilized independently.

In one embodiment the lyophilization takes place in the presence of a non-reducing sugar, possible non-reducing sugars include sucrose, trehalose, raffinose, stachyose, melezitose, dextran, mannitol, lactitol and palatinit.

The second step of the conjugation process is the reduction of the activated polysaccharide and a carrier protein to form a conjugate (so-called reductive amination), using a reducing agent. Reducing agents which are suitable include the cyanoborohydrides (such as sodium cyanoborohydride, sodium triacetoxyborohydride or sodium or zinc borohydride in the presence of Bronsted or Lewis acids), amine boranes such as pyridine borane, 2-Picoline Borane, 2,6-diborane-methanol, dimethylamine-borane, t-BuMeⁱPrN—BH₃, benzylamine-BH₃ or 5-ethyl-2-methylpyridine borane (PEMB) or borohydride exchange resin. In one embodiment the reducing agent is sodium cyanoborohydride.

In an embodiment, the reduction reaction is carried out in aqueous solvent (e.g., selected from PBS, MES, HEPES, Bis-tris, ADA, PIPES, MOPSO, BES, MOPS, DIPSO, MOBS, HEPPSO, POPSO, TEA, EPPS, Bicine or HEPB, at a pH between 6.0 and 8.5, 7.0 and 8.0, or 7.0 and 7.5), in another embodiment the reaction is carried out in aprotic solvent.

In an embodiment, the reduction reaction is carried out in DMSO (dimethylsulfoxide) or in DMF (dimethylformamide) solvent. The DMSO or DMF solvent may be used to reconstitute the activated polysaccharide and carrier protein which has been lyophilized.

At the end of the reduction reaction, there may be unreacted aldehyde groups remaining in the conjugates, these may be capped using a suitable capping agent. In one embodiment this capping agent is sodium borohydride (NaBH₄). Following the conjugation (the reduction reaction and optionally the capping), the glycoconjugates may be purified (enriched with respect to the amount of polysaccharide-protein conjugate) by a variety of techniques known to the skilled person. These techniques include dialysis, concentration/diafiltration operations, tangential flow filtration precipitation/elution, column chromatography (DEAE or hydrophobic interaction chromatography), and depth filtration. In an embodiment, the glycoconjugates are purified by diafilitration or ion exchange chromatography or size exclusion chromatography.

In one embodiment the glycoconjugates are sterile filtered.

In an embodiment of the present invention, the glycoconjugates from S. pneumoniae serotypes 1, 4, 5, 6B, 7F, 9V, 14, 18C, 19F, and 23F are prepared using CDAP chemistry and the glycoconjugate from S. pneumoniae serotype 6A is prepared by reductive amination.

In an embodiment of the present invention, the glycoconjugates from S. pneumoniae serotypes 1, 4, 5, 6B, 7F, 9V, 14, 18C, 19F, and 23F are prepared using CDAP chemistry and the glycoconjugate from S. pneumoniae serotype 19A is prepared by reductive amination.

In an embodiment of the present invention, the glycoconjugates from S. pneumoniae serotypes 1, 4, 5, 6B, 7F, 9V, 14, 18C, 19F, and 23F are prepared using CDAP chemistry and the glycoconjugates from S. pneumoniae serotype 6A and 19A are prepared by reductive amination.

In an embodiment of the present invention, the glycoconjugates from S. pneumoniae serotypes 1, 4, 5, 6B, 7F, 9V, 14, 18C, 19F, and 23F are prepared using CDAP chemistry and the glycoconjugates from S. pneumoniae serotype 3, 6A and 19A are prepared by reductive amination.

In an embodiment of the present invention, the glycoconjugates from S. pneumoniae serotypes 1, 4, 5, 6B, 7F, 9V, 14, 18C, 19F, 22F and 23F are prepared using CDAP chemistry and the glycoconjugate from S. pneumoniae serotype 6A is prepared by reductive amination.

In an embodiment of the present invention, the glycoconjugates from S. pneumoniae serotypes 1, 4, 5, 6B, 7F, 9V, 14, 18C, 19F, 22F, and 23F are prepared using CDAP chemistry and the glycoconjugate from S. pneumoniae serotype 19A is prepared by reductive amination.

In an embodiment of the present invention, the glycoconjugates from S. pneumoniae serotypes 1, 4, 5, 6B, 7F, 9V, 14, 18C, 19F, 22F, and 23F are prepared using CDAP chemistry and the glycoconjugates from S. pneumoniae serotype 6A and 19A are prepared by reductive amination.

In an embodiment of the present invention, the glycoconjugates from S. pneumoniae serotypes 1, 4, 5, 6B, 7F, 8, 9V, 14, 18C, 19F, 22F and 23F are prepared using CDAP chemistry and the glycoconjugates from S. pneumoniae serotype 3, 6A and 19A are prepared by reductive amination.

In an embodiment, the glycoconjugates of the invention are prepared using the eTEC conjugation, such as described in WO 2014/027302. Said glycoconjugates comprise a saccharide covalently conjugated to a carrier protein through one or more eTEC spacers, wherein the saccharide is covalently conjugated to the eTEC spacer through a carbamate linkage, and wherein the carrier protein is covalently conjugated to the eTEC spacer through an amide linkage. The eTEC linked glycoconjugates of the invention may be represented b the general formula (I):

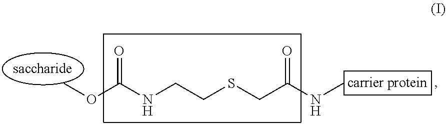

where the atoms that comprise the eTEC spacer are contained in the central box.

The eTEC spacer includes seven linear atoms (i.e., —C(O)NH(CH₂)₂SCH₂C(O)—) and provides stable thioether and amide bonds between the saccharide and carrier protein.

Synthesis of the eTEC linked glycoconjugate involves reaction of an activated hydroxyl group of the saccharide with the amino group of a thioalkylamine reagent, e.g., cystamine or cysteinamine or a salt thereof, forming a carbamate linkage to the saccharide to provide a thiolated saccharide. Generation of one or more free sulfhydryl groups is accomplished by reaction with a reducing agent to provide an activated thiolated saccharide. Reaction of the free sulfhydryl groups of the activated thiolated saccharide with an activated carrier protein having one or more α-haloacetamide groups on amine containing residues generates a thioether bond to form the conjugate, wherein the carrier protein is attached to the eTEC spacer through an amide bond.

In said glycoconjugates of the invention, the saccharide may be a polysaccharide or an oligosaccharide. The carrier protein may be selected from any suitable carrier as described herein or known to those of skill in the art. In frequent embodiments, the saccharide is a polysaccharide. In some such embodiments, the carrier protein is CRM₁₉₇.

In some such embodiments, the eTEC linked glycoconjugate comprises a S. pneumoniae serotype 33F capsular polysaccharide.

In particularly preferred embodiments, the eTEC linked glycoconjugate comprises a pneumococcal serotype 33F (Pn33F) capsular polysaccharide, which is covalently conjugated to CRM₁₉₇ through an eTEC spacer (serotype 33F eTEC linked glycoconjugates).

In some embodiments, the glycoconjugate from S. pneumoniae serotypes 1, 7F, 9V and/or 18C of the invention are O-acetylated. In some embodiments, the glycoconjugate from S. pneumoniae serotypes 1, 7F and 9V is O-acetylated and the glycoconjugate from S. pneumoniae serotype 18C is de-O-acetylated.

In some embodiments, the glycoconjugates of the present invention comprise a saccharide having a molecular weight of between 5 kDa and 2,000 kDa. In other such embodiments, the saccharide has a molecular weight of between 50 kDa and 1,000 kDa. In other such embodiments, the saccharide has a molecular weight of between 70 kDa and 900 kDa. In other such embodiments, the saccharide has a molecular weight of between 100 kDa and 800 kDa. In other such embodiments, the saccharide has a molecular weight of between 200 kDa and 600 kDa. In other such embodiments, the saccharide has a molecular weight of between 100 kDa and 500 kDa. In other such embodiments, the saccharide has a molecular weight of between 100 kDa and 400 kDa. In other such embodiments, the saccharide has a molecular weight of between 150 kDa and 300 kDa. In further embodiments, the saccharide has a molecular weight of between 5 kDa to 100 kDa; 10 kDa to 100 kDa; 20 kDa to 100 kDa; 30 kDa to 100 kDa; 40 kDa to 100 kDa; 50 kDa to 100 kDa; 60 kDa to 100 kDa; 70 kDa to 100 kDa; 80 kDa to 100 kDa; 90 kDa to 100 kDa; 5 kDa to 90 KDa; 5 kDa to 80 kDa; 5 kDa to 70 kDa; 5 kDa to 60 kDa; 5 kDa to 50 kDa; 5 kDa to 40 kDa; 5 kDa to 30 kDa; 5 kDa to 20 kDa or 5 kDa to 10 kDa.

Any whole number integer within any of the above ranges is contemplated as an embodiment of the disclosure. In some such embodiments, the glycoconjugate is prepared using reductive amination.

In some embodiments, the glycoconjugate of the invention has a molecular weight of between 100 kDa and 15,000 kDa. In some embodiments, the glycoconjugate of the invention has a molecular weight of between 500 kDa and 10,000 kDa. In some embodiments, the glycoconjugate of the invention has a molecular weight of between 2,000 kDa and 10,000 kDa. In some embodiments, the glycoconjugate of the invention has a molecular weight of between 3,000 kDa and 8,000 kDa. In some embodiments, the glycoconjugate of the invention has a molecular weight of between 3,000 kDa and 5,000 kDa. In other embodiments, the glycoconjugate has a molecular weight of between 500 kDa and 10,000 kDa. In other embodiments, glycoconjugate has a molecular weight of between 1,000 kDa and 8,000 kDa. In still other embodiments, the glycoconjugate has a molecular weight of between 2,000 kDa and 8,000 kDa or between 3,000 kDa and 7,000 kDa.

The molecular weight of the glycoconjugate is measured by SEC-MALLS. Any whole number integer within any of the above ranges is contemplated as an embodiment of the disclosure.

Another way to characterize the glycoconjugates of the invention is by the number of lysine residues in the carrier protein (e.g., CRM₁₉₇) that become conjugated to the saccharide which can be characterized as a range of conjugated lysines (degree of conjugation). The evidence for lysine modification of the carrier protein, due to covalent linkages to the polysaccharides, can be obtained by amino acid analysis using routine methods known to those of skill in the art. Conjugation results in a reduction in the number of lysine residues recovered, compared to the carrier protein starting material used to generate the conjugate materials. In a preferred embodiment, the degree of conjugation of the glycoconjugates of the invention is between 2 and 15. In an embodiment, the degree of conjugation of the glycoconjugates of the invention is between 2 and 13. In an embodiment, the degree of conjugation of the glycoconjugates of the invention is between 2 and 10. In an embodiment, the degree of conjugation of the glycoconjugates of the invention is between 2 and 8. In an embodiment, the degree of conjugation of the glycoconjugates of the invention is between 2 and 6. In an embodiment, the degree of conjugation of the glycoconjugates of the invention is between 3 and 10. In an embodiment, the degree of conjugation of the glycoconjugates of the invention is between 3 and 6. In an embodiment, the degree of conjugation of the glycoconjugates of the invention is between 5 and 10. In an embodiment, the degree of conjugation of the glycoconjugates of the invention is between 8 and 12. In an embodiment, the degree of conjugation of the glycoconjugate of the invention is about 2. In an embodiment, the degree of conjugation of the glycoconjugate of the invention is about 3. In an embodiment, the degree of conjugation of the glycoconjugate of the invention is about 4. In an embodiment, the degree of conjugation of the glycoconjugate of the invention is about 5.

In an embodiment, the degree of conjugation of the glycoconjugate of the invention is about 6. In an embodiment, the degree of conjugation of the glycoconjugate of the invention is about 8. In an embodiment, the degree of conjugation of the glycoconjugate of the invention is about 10. In an embodiment, the degree of conjugation of the glycoconjugate of the invention is about 12. In an embodiment, the degree of conjugation of the glycoconjugate of the invention is about 15. In a preferred embodiment, the degree of conjugation of the glycoconjugate of the invention is between 4 and 7. In some such embodiments, the carrier protein is CRM₁₉₇.

The glycoconjugates of the invention may also be characterized by the ratio (weight/weight) of saccharide to carrier protein. In some embodiments, the ratio of polysaccharide to carrier protein in the glycoconjugate (w/w) is between 0.5 and 3. In some embodiments, the ratio of polysaccharide to carrier protein in the glycoconjugate (w/w) is about 0.8. In some embodiments, the ratio of polysaccharide to carrier protein in the glycoconjugate (w/w) is about 0.9. In some embodiments, the ratio of polysaccharide to carrier protein in the glycoconjugate (w/w) is about 1.0. In some embodiments, the ratio of polysaccharide to carrier protein in the glycoconjugate (w/w) is about 1.2. In some embodiments, the ratio of polysaccharide to carrier protein in the glycoconjugate (w/w) is about 1.5. In some embodiments, the ratio of polysaccharide to carrier protein in the glycoconjugate (w/w) is about 1.8. In some embodiments, the ratio of polysaccharide to carrier protein in the glycoconjugate (w/w) is about 2.0. In some embodiments, the ratio of polysaccharide to carrier protein in the glycoconjugate (w/w) is about 2.5. In some embodiments, the ratio of polysaccharide to carrier protein in the glycoconjugate (w/w) is about 3.0. In other embodiments, the saccharide to carrier protein ratio (w/w) is between 0.5 and 2.0. In other embodiments, the saccharide to carrier protein ratio (w/w) is between 0.5 and 1.5. In further embodiments, the saccharide to carrier protein ratio (w/w) is between 0.8 and 1.2. In a preferred embodiment, the ratio of capsular polysaccharide to carrier protein in the conjugate is between 0.9 and 1.1. In some such embodiments, the carrier protein is CRM₁₉₇.

The glycoconjugates and immunogenic compositions of the invention may contain free saccharide that is not covalently conjugated to the carrier protein, but is nevertheless present in the glycoconjugate composition. The free saccharide may be non-covalently associated with (i.e., non-covalently bound to, adsorbed to, or entrapped in or with) the glycoconjugate.

In a preferred embodiment, the glycoconjugate comprises less than about 50%, 45%, 40%, 35%, 30%, 25%, 20% or 15% of free polysaccharide compared to the total amount of polysaccharide. In a preferred embodiment the glycoconjugate comprises less than about 25% of free polysaccharide compared to the total amount of polysaccharide. In a preferred embodiment the glycoconjugate comprises less than about 20% of free polysaccharide compared to the total amount of polysaccharide. In a preferred embodiment the glycoconjugate comprises less than about 15% of free polysaccharide compared to the total amount of polysaccharide.

The glycoconjugates may also be characterized by their molecular size distribution (K_{d}). Size exclusion chromatography media (CL-4B) can be used to determine the relative molecular size distribution of the conjugate. Size Exclusion Chromatography (SEC) is used in gravity fed columns to profile the molecular size distribution of conjugates. Large molecules excluded from the pores in the media elute more quickly than small molecules. Fraction collectors are used to collect the column eluate. The fractions are tested colorimetrically by saccharide assay. For the determination of K_{d}, columns are calibrated to establish the fraction at which molecules are fully excluded (V₀), (K_{d}=0), and the fraction representing the maximum retention (Vᵢ), (K_{d}=1). The fraction at which a specified sample attribute is reached (Vₑ), is related to K_{d} by the expression, K_{d}=(Vₑ−V₀)/(Vᵢ−V₀).

In a preferred embodiment, at least 30% of the glycoconjugate has a K_{d} below or equal to 0.3 in a CL-4B column. In a preferred embodiment, at least 40% of the glycoconjugate has a K_{d} below or equal to 0.3 in a CL-4B column. In a preferred embodiment, at least 45%, 50%, 55%, 60%, 65%, 70%, 75%, 80%, or 85% of the glycoconjugate has a K_{d} below or equal to 0.3 in a CL-4B column. In a preferred embodiment, at least 60% of the glycoconjugate has a K_{d} below or equal to 0.3 in a CL-4B column. In a preferred embodiment, between 50% and 80% of the glycoconjugate has a K_{d} below or equal to 0.3 in a CL-4B column. In a preferred embodiment, between 65% and 80% of the glycoconjugate has a K_{d} below or equal to 0.3 in a CL-4B column.

The frequency of attachment of the saccharide chain to a lysine on the carrier protein is another parameter for characterizing the glycoconjugates of the invention. For example, in some embodiments, at least one covalent linkage between the carrier protein and the polysaccharide occurs for every 4 saccharide repeat units of the polysaccharide. In another embodiment, the covalent linkage between the carrier protein and the polysaccharide occurs at least once in every 10 saccharide repeat units of the polysaccharide. In another embodiment, the covalent linkage between the carrier protein and the polysaccharide occurs at least once in every 15 saccharide repeat units of the polysaccharide. In a further embodiment, the covalent linkage between the carrier protein and the polysaccharide occurs at least once in every 25 saccharide repeat units of the polysaccharide.

In frequent embodiments, the carrier protein is CRM₁₉₇ and the covalent linkage via an eTEC spacer between the CRM₁₉₇ and the polysaccharide occurs at least once in every 4, 10, 15 or 25 saccharide repeat units of the polysaccharide.

In other embodiments, the conjugate comprises at least one covalent linkage between the carrier protein and saccharide for every 5 to 10 saccharide repeat units. In other embodiments, the conjugate comprises at least one covalent linkage between the carrier protein and saccharide every 2 to 7 saccharide repeat units. In other embodiments, the conjugate comprises at least one covalent linkage between the carrier protein and saccharide for every 7 to 12 saccharide repeat units. In other embodiments, the conjugate comprises at least one covalent linkage between the carrier protein and saccharide for every 10 to 15 saccharide repeat units. In other embodiments, the conjugate comprises at least one covalent linkage between the carrier protein and saccharide for every 4 to 8 saccharide repeat units. In other embodiments, the conjugate comprises at least one covalent linkage between the carrier protein and saccharide for every 10 to 20 saccharide repeat units In other embodiments, the conjugate comprises at least one covalent linkage between the carrier protein and saccharide for every 2 to 25 saccharide repeat units. In frequent embodiments, the carrier protein is CRM₁₉₇.

In another embodiment, at least one linkage between carrier protein and saccharide occurs for every 2, 3, 4, 5, 6, 7, 8, 9, 10, 11, 12, 13, 14, 15, 16, 17, 18, 19, 20, 21, 22, 23, 24 or 25 saccharide repeat units of the polysaccharide. In an embodiment, the carrier protein is CRM₁₉₇. Any whole number integer within any of the above ranges is contemplated as an embodiment of the disclosure.

1.4 Carrier Protein of the Invention

A component of the glycoconjugate of the invention is a carrier protein to which the saccharide is conjugated. The terms “protein carrier” or “carrier protein” or “carrier” may be used interchangeably herein. Carrier proteins should be amenable to standard conjugation procedures.

In a preferred embodiment, the carrier protein of the glycoconjugates is selected in the group consisting of: DT (Diphtheria toxin), TT (tetanus toxoid) or fragment C of TT, CRM₁₉₇ (a nontoxic but antigenically identical variant of diphtheria toxin), the A chain of diphtheria toxin mutant CRM₁₉₇ (CN103495161), other DT mutants (such as CRM176, CRM228, CRM45 (Uchida et al. (1973) J. Biol. Chem. 218:3838-3844), CRM9, CRM102, CRM103 or CRM107; and other mutations described by Nicholls and Youle in Genetically Engineered Toxins, Ed: Frankel, Maecel Dekker Inc. (1992); deletion or mutation of Glu-148 to Asp, Gln or Ser and/or Ala 158 to Gly and other mutations disclosed in U.S. Pat. Nos. 4,709,017 and 4,950,740; mutation of at least one or more residues Lys 516, Lys 526, Phe 530 and/or Lys 534 and other mutations disclosed in U.S. Pat. Nos. 5,917,017 and 6,455,673; or fragment disclosed in U.S. Pat. No. 5,843,711, pneumococcal pneumolysin (ply) (Kuo et al. (1995) Infect Immun 63:2706-2713) including ply detoxified in some fashion, for example dPLY-GMBS (WO 2004/081515 and WO 2006/032499) or dPLY-formol, PhtX, including PhtA, PhtB, PhtD, PhtE (sequences of PhtA, PhtB, PhtD or PhtE are disclosed in WO 00/37105 and WO 00/39299) and fusions of Pht proteins for example PhtDE fusions, PhtBE fusions, Pht A-E (WO 01/98334, WO 03/054007, WO 2009/000826), OMPC (meningococcal outer membrane protein—usually extracted from Neisseria meningitidis serogroup B (EP0372501), PorB (from N. meningitidis), PD (Haemophilus influenzae protein D; see, e.g., EP0594610 B), or immunologically functional equivalents thereof, synthetic peptides (EP0378881, EP0427347), heat shock proteins (WO 93/17712, WO 94/03208), pertussis proteins (WO 98/58668, EP0471177), cytokines, lymphokines, growth factors or hormones (WO 91/01146), artificial proteins comprising multiple human CD4+ T cell epitopes from various pathogen derived antigens (Falugi et al. (2001) Eur J Immunol 31:3816-3824) such as N19 protein (Baraldoi et al. (2004) Infect Immun 72:4884-4887) pneumococcal surface protein PspA (WO 02/091998), iron uptake proteins (WO 01/72337), toxin A or B of Clostridium difficile (WO 00/61761), transferrin binding proteins, pneumococcal adhesion protein (PsaA), recombinant Pseudomonas aeruginosa exotoxin A (in particular non-toxic mutants thereof (such as exotoxin A bearing a substitution at glutamic acid 553 (Douglas et al. (1987) J. Bacteriol. 169(11):4967-4971)). Other proteins, such as ovalbumin, keyhole limpet hemocyanin (KLH), bovine serum albumin (BSA) or purified protein derivative of tuberculin (PPD) also can be used as carrier proteins. Other suitable carrier proteins include inactivated bacterial toxins such as cholera toxoid (e.g., as described in WO 2004/083251), Escherichia coli LT, E. coli ST, and exotoxin A from P. aeruginosa. Another suitable carrier protein is a C5a peptidase from Streptococcus (SCP).

In a preferred embodiment, the carrier protein of the glycoconjugates is independently selected from the group consisting of TT, DT, DT mutants (such as CRM₁₉₇), H. influenzae protein D, PhtX, PhtD, PhtDE fusions (particularly those described in WO 01/98334 and WO 03/054007), detoxified pneumolysin, PorB, N19 protein, PspA, OMPC, toxin A or B of C. difficile and PsaA.

In an embodiment, the carrier protein of the glycoconjugates of the invention is DT (Diphtheria toxoid). In another embodiment, the carrier protein of the glycoconjugates of the invention is TT (tetanus toxoid). In another embodiment, the carrier protein of the glycoconjugates of the invention is a C5a peptidase from Streptococcus (SCP). In an embodiment, the carrier protein of the glycoconjugates of the invention is an enzymatically inactive streptococcal C5a peptidase (SCP).

In another embodiment, the carrier protein of the glycoconjugates of the invention is PD (H. influenzae protein D; see, e.g., EP0594610 B).

The CRM₁₉₇ protein is a nontoxic form of diphtheria toxin but is immunologically indistinguishable from the diphtheria toxin. CRM₁₉₇ is produced by Corynebacterium diphtheriae infected by the nontoxigenic phage β197^{tox-} created by nitrosoguanidine mutagenesis of the toxigenic corynephage beta (Uchida et al. (1971) Nature New Biology 233:8-11). The CRM₁₉₇ protein has the same molecular weight as the diphtheria toxin but differs therefrom by a single base change (guanine to adenine) in the structural gene. This single base change causes an amino acid substitution (glutamic acid for glycine) in the mature protein and eliminates the toxic properties of diphtheria toxin. The CRM₁₉₇ protein is a safe and effective T-cell dependent carrier for saccharides. Further details about CRM₁₉₇ and production thereof can be found, e.g., in U.S. Pat. No. 5,614,382. In an embodiment, the capsular saccharides of the invention are conjugated to CRM₁₉₇ protein or the A chain of CRM₁₉₇ (see CN103495161). In an embodiment, the capsular saccharides of the invention are conjugated the A chain of CRM₁₉₇ obtained via expression by genetically recombinant E. coli (see CN103495161). In a preferred embodiment, the capsular saccharides of the invention are conjugated to CRM₁₉₇. In an embodiment, the capsular saccharides of the invention are all conjugated to CRM₁₉₇.

Accordingly, in frequent embodiments, the glycoconjugates of the invention comprise CRM₁₉₇ as the carrier protein, wherein the capsular polysaccharide is covalently linked to CRM₁₉₇.

1.5 Serotype 15B Glycoconjugates of the Invention

In one embodiment, the 15B glycoconjugates of the invention are as defined in the present section.

Capsular polysaccharides from serotype 15B of S. pneumoniae are prepared as disclosed above (see also WO2015/110942).

In an embodiment, the polysaccharides are activated with 1-cyano-4-dimethylamino pyridinium tetrafluoroborate (CDAP) to form a cyanate ester. The activated polysaccharide is then coupled directly or via a spacer (linker) group to an amino group on the carrier protein (preferably CRM₁₉₇). For example, the spacer could be cystamine or cysteamine to give a thiolated polysaccharide which could be coupled to the carrier via a thioether linkage obtained after reaction with a maleimide-activated carrier protein (for example using N-[γ-maleimidobutyrloxy]succinimide ester (GMBS)) or a haloacetylated carrier protein (for example using iodoacetimide, N-succinimidyl bromoacetate (SBA; SIB), N-succinimidyl(4-iodoacetyl)aminobenzoate (SIAB), sulfosuccinimidyl(4-iodoacetyl)aminobenzoate (sulfo-SIAB), N-succinimidyl iodoacetate (SIA), or succinimidyl 3-[bromoacetamido]proprionate (SBAP)). Preferably, the cyanate ester (optionally made by CDAP chemistry) is coupled with hexane diamine or adipic acid dihydrazide (ADH) and the amino-derivatised saccharide is conjugated to the carrier protein (e.g., CRM₁₉₇) using carbodiimide (e.g., EDAC or EDC) chemistry via a carboxyl group on the protein carrier. Such conjugates are described for example in WO 93/15760, WO 95/08348 and WO 96/129094.

In an embodiment of the present invention, the glycoconjugate from S. pneumoniae serotype 15B is prepared using CDAP chemistry.

Other suitable techniques for conjugation use carbodiimides, hydrazides, active esters, norborane, p-nitrobenzoic acid, N-hydroxysuccinimide, S—NHS, EDC, TSTU. Many are described in International Patent Application Publication No. WO 98/42721. Conjugation may involve a carbonyl linker which may be formed by reaction of a free hydroxyl group of the saccharide with CDI (see Bethell et al. (1979) 1. Biol. Chern. 254:2572-2574; Hearn et al. (1981) J. Chromatogr. 218:509-518) followed by reaction with a protein to form a carbamate linkage. This may involve reduction of the anomeric terminus to a primary hydroxyl group, optional protection/deprotection of the primary hydroxyl group, reaction of the primary hydroxyl group with CDI to form a CDI carbamate intermediate and coupling the CDI carbamate intermediate with an amino group on a protein.

In a preferred embodiment, capsular polysaccharide from serotype 15B of S. pneumoniae is conjugated to the carrier protein by reductive amination (such as described in U.S. Patent Appl. Pub. Nos. 2006/0228380, 2007/184072, 2007/0231340 and 2007/0184071, WO 2006/110381, WO 2008/079653, WO 2008/143709, WO2015/110942 and WO2019/139692). Therefore, in a preferred embodiment, the serotype 15B glycoconjugate of the present invention is prepared by reductive amination.

Reductive amination involves two steps as disclosed above.

In some embodiments, the glycoconjugate from S. pneumoniae serotype 15B of the present invention comprises a saccharide having a molecular weight of between 5 kDa and 2,000 kDa. In other such embodiments, the saccharide has a molecular weight of between 20 kDa and 800 kDa. In further such embodiments, the saccharide has a molecular weight of between 100 kDa to 1000 kDa. In further such embodiments, the saccharide has a molecular weight of between 100 kDa to 800 kDa. In further such embodiments, the saccharide has a molecular weight of between 100 kDa to 500 kDa. In further such embodiments, the saccharide has a molecular weight of between 100 kDa to 400 kDa. In further such embodiments, the saccharide has a molecular weight of between 100 kDa to 300 kDa. In a preferred embodiment, the saccharide has a molecular weight of between 100 kDa to 350 kDa. In another preferred embodiment, the saccharide has a molecular weight of between 100 kDa to 300 kDa. In a more preferred embodiment, the saccharide has a molecular weight of between 150 kDa to 300 kDa.

In further embodiments, the saccharide has a molecular weight of between 5 kDa to 100 kDa; 10 kDa to 100 kDa; 20 kDa to 100 kDa; 50 kDa to 100 kDa or 90 kDa to 100 kDa.

Any whole number integer within any of the above ranges is contemplated as an embodiment of the disclosure. In some such embodiments, the glycoconjugate is prepared using reductive amination.

In some embodiments, the glycoconjugate from S. pneumoniae serotype 15B of the present invention has a molecular weight of between 100 kDa and 20,000 kDa. In other such embodiments, the glycoconjugate from S. pneumoniae serotype 15B of the present invention has a molecular weight of between 200 kDa and 10,000 kDa. In other such embodiments, the glycoconjugate from S. pneumoniae serotype 15B of the present invention has a molecular weight of between 2000 kDa and 5,000 kDa. In further embodiments, the glycoconjugate from S. pneumoniae serotype 15B of the invention has a molecular weight of between 1000 kDa and 5,000 kDa. In further embodiments, the glycoconjugate from S. pneumoniae serotype 15B of the invention has a molecular weight of between 1500 kDa and 4,500 kDa. In further embodiments, the glycoconjugate from S. pneumoniae serotype 15B of the invention has a molecular weight of between 2000 kDa and 4,500 kDa. In further embodiments, the glycoconjugate from S. pneumoniae serotype 15B of the invention has a molecular weight of between 500 kDa and 2,000 kDa. The molecular weight of the glycoconjugate is measured by SEC-MALLS. Any whole number integer within any of the above ranges is contemplated as an embodiment of the disclosure.

Another way to characterize the glycoconjugates of the invention is by the number of lysine residues in the carrier protein (e.g., CRM₁₉₇) that become conjugated to the saccharide which can be characterized as a range of conjugated lysines (degree of conjugation). The evidence for lysine modification of the carrier protein, due to covalent linkages to the polysaccharides, can be obtained by amino acid analysis using routine methods known to those of skill in the art. Conjugation results in a reduction in the number of lysine residues recovered, compared to the carrier protein starting material used to generate the conjugate materials. In a preferred embodiment, the degree of conjugation of the 15B glycoconjugates of the invention is between 2 and 15. In an embodiment, the degree of conjugation of the 15B glycoconjugates of the invention is between 2 and 13. In an embodiment, the degree of conjugation of the 15B glycoconjugates of the invention is between 2 and 10. In an embodiment, the degree of conjugation of the 15B glycoconjugates of the invention is between 2 and 8. In an embodiment, the degree of conjugation of the 15B glycoconjugates of the invention is between 2 and 6. In an embodiment, the degree of conjugation of the 15B glycoconjugates of the invention is between 3 and 10. In an embodiment, the degree of conjugation of the 15B glycoconjugates of the invention is between 3 and 6. In an embodiment, the degree of conjugation of the 15B glycoconjugates of the invention is between 5 and 10. In an embodiment, the degree of conjugation of the 15B glycoconjugates of the invention is between 8 and 12. In a preferred embodiment, the degree of conjugation of the 15B glycoconjugates of the invention is between 2 and 8. In an embodiment, the degree of conjugation of the glycoconjugate of the invention is about 2. In an embodiment, the degree of conjugation of the glycoconjugate of the invention is about 3. In an embodiment, the degree of conjugation of the glycoconjugate of the invention is about 4. In an embodiment, the degree of conjugation of the glycoconjugate of the invention is about 5. In an embodiment, the degree of conjugation of the glycoconjugate of the invention is about 6. In an embodiment, the degree of conjugation of the glycoconjugate of the invention is about 8. In an embodiment, the degree of conjugation of the glycoconjugate of the invention is about 10. In an embodiment, the degree of conjugation of the glycoconjugate of the invention is about 12. In an embodiment, the degree of conjugation of the glycoconjugate of the invention is about 15. In a preferred embodiment, the degree of conjugation of the 15B glycoconjugate of the invention is between 4 and 7. In some such embodiments, the carrier protein is CRM₁₉₇.

The glycoconjugates of the invention may also be characterized by the ratio (weight/weight) of saccharide to carrier protein. In some embodiments, the ratio of polysaccharide to carrier protein in the 15B glycoconjugate (w/w) is between 0.5 and 3. In some embodiments, the ratio of polysaccharide to carrier protein in the 15B glycoconjugate (w/w) is between 0.4 and 2. In some embodiments, the ratio of polysaccharide to carrier protein in the 15B glycoconjugate (w/w) is about 0.8. In some embodiments, the ratio of polysaccharide to carrier protein in the 15B glycoconjugate (w/w) is about 0.9. In some embodiments, the ratio of polysaccharide to carrier protein in the 15B glycoconjugate (w/w) is about 1.0. In some embodiments, the ratio of polysaccharide to carrier protein in the 15B glycoconjugate (w/w) is about 1.2. In some embodiments, the ratio of polysaccharide to carrier protein in the 15B glycoconjugate (w/w) is about 1.5. In some embodiments, the ratio of polysaccharide to carrier protein in the 15B glycoconjugate (w/w) is about 1.8. In some embodiments, the ratio of polysaccharide to carrier protein in the 15B glycoconjugate (w/w) is about 2.0. In some embodiments, the ratio of polysaccharide to carrier protein in the 15B glycoconjugate (w/w) is about 2.5. In some embodiments, the ratio of polysaccharide to carrier protein in the 15B glycoconjugate (w/w) is about 3.0. In other embodiments, the saccharide to carrier protein ratio (w/w) is between 0.5 and 2.0. In other embodiments, the saccharide to carrier protein ratio (w/w) is between 0.5 and 1.5. In further embodiments, the saccharide to carrier protein ratio (w/w) is between 0.8 and 1.2. In a preferred embodiment, the ratio of capsular polysaccharide to carrier protein in the conjugate is between 0.9 and 1.1. In some such embodiments, the carrier protein is CRM₁₉₇.

The frequency of attachment of the saccharide chain to a lysine on the carrier protein is another parameter for characterizing the glycoconjugate from S. pneumoniae serotype 15B of the invention. For example, in some embodiments, at least one covalent linkage between the carrier protein and the 15B saccharide occurs for every 4 saccharide repeat units of the 15B saccharide. In another embodiment, the covalent linkage between the carrier protein and the 15B saccharide occurs at least once in every 10 saccharide repeat units of the 15B saccharide. In another embodiment, the covalent linkage between the carrier protein and the 15B saccharide occurs at least once in every 15 saccharide repeat units of the 15B saccharide. In a further embodiment, the covalent linkage between the carrier protein and the 15B saccharide occurs at least once in every 25 saccharide repeat units of the 15B saccharide.

In other embodiments, the 15B glycoconjugate comprises at least one covalent linkage between the carrier protein and 15B saccharide for every 5 to 10 saccharide repeat units. In other embodiments, the 15B glycoconjugate comprises at least one covalent linkage between the carrier protein and 15B saccharide every 2 to 7 saccharide repeat units. In other embodiments, the 15B glycoconjugate comprises at least one covalent linkage between the carrier protein and 15B saccharide for every 7 to 12 saccharide repeat units. In other embodiments, the 15B glycoconjugate comprises at least one covalent linkage between the carrier protein and 15B saccharide for every 10 to 15 saccharide repeat units. In other embodiments, the 15B glycoconjugate comprises at least one covalent linkage between the carrier protein and saccharide 15B saccharide for every 4 to 8 saccharide repeat units. In other embodiments, the 15B glycoconjugate comprises at least one covalent linkage between the carrier protein and 15B saccharide for every 10 to 20 saccharide repeat units. In other embodiments, the 15B glycoconjugate comprises at least one covalent linkage between the carrier protein and 15B saccharide for every 2 to 25 saccharide repeat units. In frequent embodiments, the carrier protein is CRM₁₇.

In another embodiment, at least one linkage between carrier protein and saccharide occurs for every 2, 3, 4, 5, 6, 7, 8, 9, 10, 11, 12, 13, 14, 15, 16, 17, 18, 19, 20, 21, 22, 23, 24 or 25 saccharide repeat units of the polysaccharide. In an embodiment, the carrier protein is CRM₁₉₇. Any whole number integer within any of the above ranges is contemplated as an embodiment of the disclosure.

In a preferred embodiment, the serotype 15B glycoconjugate of the invention comprises at least 0.5 mM acetate per mM serotype 15B capsular polysaccharide. In an embodiment, the serotype 15B glycoconjugate of the invention comprises at least 0.6 mM acetate per mM serotype 15B capsular polysaccharide. In an embodiment, the serotype 15B glycoconjugate of the invention comprises at least 0.65 mM acetate per mM serotype 15B capsular polysaccharide. In a preferred embodiment, the glycoconjugate comprises at least 0.7 mM acetate per mM serotype 15B capsular polysaccharide. In an embodiment, the serotype 15B glycoconjugate of the invention comprises between 0.5 mM and 0.85 mM acetate per mM serotype 15B capsular polysaccharide. In an embodiment, the serotype 15B glycoconjugate of the invention comprises between 0.6 mM and 0.85 mM acetate per mM serotype 15B capsular polysaccharide. In a preferred embodiment, the serotype 15B glycoconjugate of the invention comprises between 0.7 mM and 0.85 mM acetate per mM serotype 15B capsular polysaccharide. In an embodiment, the serotype 15B glycoconjugate of the invention comprises between 0.8 mM and 0.85 mM acetate per mM serotype 15B capsular polysaccharide. In a preferred embodiment, the presence of 0-acetyl groups is determined by ion-HPLC analysis.

In an embodiment, the serotype 15B glycoconjugate of the invention comprises at least 0.1 mM glycerol per mM serotype 15B capsular polysaccharide. In an embodiment, the serotype 15B glycoconjugate of the invention comprises at least 0.2 mM glycerol per mM serotype 15B capsular polysaccharide. In an embodiment, the serotype 15B glycoconjugate of the invention comprises at least 0.5 mM glycerol per mM serotype 15B capsular polysaccharide. In an embodiment, the serotype 15B glycoconjugate of the invention comprises at least 0.8 mM glycerol per mM serotype 15B capsular polysaccharide. In an embodiment, the serotype 15B glycoconjugate of the invention comprises at least 0.9 mM glycerol per mM serotype 15B capsular polysaccharide. In a preferred embodiment, the serotype 15B glycoconjugate of the invention comprises between 0.6 mM to 1 mM glycerol per mM serotype 15B capsular polysaccharide. In an even preferred embodiment, the serotype 15B glycoconjugate of the invention comprises between 0.7 mM to 1 mM glycerol per mM serotype 15B capsular polysaccharide. In a most preferred embodiment, the serotype 15B glycoconjugate of the invention comprises between 0.8 mM to 1 mM glycerol per mM serotype 15B capsular polysaccharide.

In an embodiment, the serotype 15B glycoconjugate of the invention comprises between 0.5 mM to 0.7 mM glycerol per mM serotype 15B capsular polysaccharide. In an even preferred embodiment, the serotype 15B glycoconjugate of the invention comprises about 0.7 mM glycerol per mM serotype 15B capsular polysaccharide.

In a preferred embodiment, the glycoconjugate from S. pneumoniae serotype 15B of the invention comprises less than about 50%, 45%, 40%, 35%, 30%, 25%, 20% or 15% of free polysaccharide compared to the total amount of polysaccharide. In an embodiment the 15B glycoconjugate comprises less than about 25% of free polysaccharide compared to the total amount of polysaccharide. In an embodiment the 15B glycoconjugate comprises less than about 20% of free polysaccharide compared to the total amount of polysaccharide. In a preferred embodiment the 15B glycoconjugate comprises less than about 15% of free polysaccharide compared to the total amount of polysaccharide.

The glycoconjugates may also be characterized by their molecular size distribution (K_{d}). Size exclusion chromatography media (CL-4B) can be used to determine the relative molecular size distribution of the conjugate. Size Exclusion Chromatography (SEC) is used in gravity fed columns to profile the molecular size distribution of conjugates. Large molecules excluded from the pores in the media elute more quickly than small molecules. Fraction collectors are used to collect the column eluate. The fractions are tested colorimetrically by saccharide assay. For the determination of K_{d}, columns are calibrated to establish the fraction at which molecules are fully excluded (V₀), (K_{d}=0), and the fraction representing the maximum retention (Vᵢ), (K_{d}=1). The fraction at which a specified sample attribute is reached (Vₑ), is related to K_{d} by the expression, K_{d}=(Vₑ−V₀)/(Vᵢ−V₀).

In a preferred embodiment, at least 30% of the glycoconjugate from S. pneumoniae serotype 15B of the invention has a K_{d} below or equal to 0.3 in a CL-4B column. In an embodiment, at least 40% of the 15B glycoconjugate has a K_{d} below or equal to 0.3 in a CL-4B column. In a preferred embodiment, at least 45%, 50%, 55%, 60%, 65%, 70%, 75%, 80%, or 85% of the 15B glycoconjugate has a K_{d} below or equal to 0.3 in a CL-4B column. In an embodiment, at least 60% of the 15B glycoconjugate has a K_{d} below or equal to 0.3 in a CL-4B column. In an embodiment, between 50% and 80% of the 15B glycoconjugate has a K_{d} below or equal to 0.3 in a CL-4B column. In a preferred embodiment, between 65% and 80% of the 15B glycoconjugate has a K_{d} below or equal to 0.3 in a CL-4B column.

1.6 Serotype 15C Glycoconjugates of the Invention

In one embodiment, the 15C glycoconjugates of the invention are as defined in the present section.

Capsular polysaccharides from serotype 15C of S. pneumoniae are prepared as disclosed above (see also WO2019/139692).

In an embodiment, the polysaccharides are activated with 1-cyano-4-dimethylamino pyridinium tetrafluoroborate (CDAP) to form a cyanate ester. The activated polysaccharide is then coupled directly or via a spacer (linker) group to an amino group on the carrier protein (preferably CRM₁₉₇). For example, the spacer could be cystamine or cysteamine to give a thiolated polysaccharide which could be coupled to the carrier via a thioether linkage obtained after reaction with a maleimide-activated carrier protein (for example using N-[γ-maleimidobutyrloxy]succinimide ester (GMBS)) or a haloacetylated carrier protein (for example using iodoacetimide, N-succinimidyl bromoacetate (SBA; SIB), N-succinimidyl(4-iodoacetyl)aminobenzoate (SIAB), sulfosuccinimidyl(4-iodoacetyl)aminobenzoate (sulfo-SIAB), N-succinimidyl iodoacetate (SIA), or succinimidyl 3-[bromoacetamido]proprionate (SBAP)). Preferably, the cyanate ester (optionally made by CDAP chemistry) is coupled with hexane diamine or adipic acid dihydrazide (ADH) and the amino-derivatised saccharide is conjugated to the carrier protein (e.g., CRM₁₉₇) using carbodiimide (e.g., EDAC or EDC) chemistry via a carboxyl group on the protein carrier. Such conjugates are described for example in WO 93/15760, WO 95/08348 and WO 96/129094.

In an embodiment of the present invention, the glycoconjugate from S. pneumoniae serotype 15C is prepared using CDAP chemistry.

Other suitable techniques for conjugation use carbodiimides, hydrazides, active esters, norborane, p-nitrobenzoic acid, N-hydroxysuccinimide, S—NHS, EDC, TSTU. Many are described in International Patent Application Publication No. WO 98/42721. Conjugation may involve a carbonyl linker which may be formed by reaction of a free hydroxyl group of the saccharide with CDI (see Bethell et al. (1979) 1. Biol. Chern. 254:2572-2574; Hearn et al. (1981) J. Chromatogr. 218:509-518) followed by reaction with a protein to form a carbamate linkage. This may involve reduction of the anomeric terminus to a primary hydroxyl group, optional protection/deprotection of the primary hydroxyl group, reaction of the primary hydroxyl group with CDI to form a CDI carbamate intermediate and coupling the CDI carbamate intermediate with an amino group on a protein.

In a preferred embodiment, capsular polysaccharide from serotype 15C of S. pneumoniae is conjugated to the carrier protein by reductive amination (such as described in U.S. Patent Appl. Pub. Nos. 2006/0228380, 2007/184072, 2007/0231340 and 2007/0184071, WO 2006/110381, WO 2008/079653, WO 2008/143709, WO2015/110942 and WO2019/139692). Therefore, in a preferred embodiment, the serotype 15C glycoconjugate of the present invention is prepared by reductive amination.

Reductive amination involves two steps as disclosed above.

In some embodiments, the glycoconjugate from S. pneumoniae serotype 15C of the present invention comprises a saccharide having a molecular weight of between 5 kDa and 2,000 kDa. In other such embodiments, the saccharide has a molecular weight of between 20 kDa and 800 kDa. In further such embodiments, the saccharide has a molecular weight of between 100 kDa to 1000 kDa. In further such embodiments, the saccharide has a molecular weight of between 100 kDa to 800 kDa. In further such embodiments, the saccharide has a molecular weight of between 100 kDa to 500 kDa. In further such embodiments, the saccharide has a molecular weight of between 100 kDa to 400 kDa. In further such embodiments, the saccharide has a molecular weight of between 100 kDa to 300 kDa. In a preferred embodiment, the saccharide has a molecular weight of between 100 kDa to 350 kDa. In another preferred embodiment, the saccharide has a molecular weight of between 100 kDa to 300 kDa. In a more preferred embodiment, the saccharide has a molecular weight of between 150 kDa to 300 kDa.

In further embodiments, the saccharide has a molecular weight of between 5 kDa to 100 kDa; 10 kDa to 100 kDa; 20 kDa to 100 kDa; 50 kDa to 100 kDa or 90 kDa to 100 kDa. Any whole number integer within any of the above ranges is contemplated as an embodiment of the disclosure. In some such embodiments, the glycoconjugate is prepared using reductive amination.

In some embodiments, the glycoconjugate from S. pneumoniae serotype 15C of the present invention has a molecular weight of between 100 kDa and 20,000 kDa. In other such embodiments, the glycoconjugate from S. pneumoniae serotype 15C of the present invention has a molecular weight of between 200 kDa and 10,000 kDa. In other such embodiments, the glycoconjugate from S. pneumoniae serotype 15C of the present invention has a molecular weight of between 2000 kDa and 5,000 kDa. In further embodiments, the glycoconjugate from S. pneumoniae serotype 15C of the invention has a molecular weight of between 1000 kDa and 5,000 kDa. In further embodiments, the glycoconjugate from S. pneumoniae serotype 15C of the invention has a molecular weight of between 1500 kDa and 4,500 kDa. In further embodiments, the glycoconjugate from S. pneumoniae serotype 15C of the invention has a molecular weight of between 2000 kDa and 4,500 kDa. In further embodiments, the glycoconjugate from S. pneumoniae serotype 15C of the invention has a molecular weight of between 500 kDa and 2,000 kDa. The molecular weight of the glycoconjugate is measured by SEC-MALLS. Any whole number integer within any of the above ranges is contemplated as an embodiment of the disclosure.

Another way to characterize the glycoconjugates of the invention is by the number of lysine residues in the carrier protein (e.g., CRM₁₉₇) that become conjugated to the saccharide which can be characterized as a range of conjugated lysines (degree of conjugation). The evidence for lysine modification of the carrier protein, due to covalent linkages to the polysaccharides, can be obtained by amino acid analysis using routine methods known to those of skill in the art. Conjugation results in a reduction in the number of lysine residues recovered, compared to the carrier protein starting material used to generate the conjugate materials. In a preferred embodiment, the degree of conjugation of the 15C glycoconjugates of the invention is between 2 and 15. In an embodiment, the degree of conjugation of the 15C glycoconjugates of the invention is between 2 and 13. In an embodiment, the degree of conjugation of the 15C glycoconjugates of the invention is between 2 and 10. In an embodiment, the degree of conjugation of the 15C glycoconjugates of the invention is between 2 and 8. In an embodiment, the degree of conjugation of the 15C glycoconjugates of the invention is between 2 and 6. In an embodiment, the degree of conjugation of the 15C glycoconjugates of the invention is between 3 and 10. In an embodiment, the degree of conjugation of the 15C glycoconjugates of the invention is between 3 and 6. In an embodiment, the degree of conjugation of the 15C glycoconjugates of the invention is between 5 and 10. In an embodiment, the degree of conjugation of the 15C glycoconjugates of the invention is between 8 and 12. In a preferred embodiment, the degree of conjugation of the 15C glycoconjugates of the invention is between 2 and 8. In an embodiment, the degree of conjugation of the glycoconjugate of the invention is about 2. In an embodiment, the degree of conjugation of the glycoconjugate of the invention is about 3. In an embodiment, the degree of conjugation of the glycoconjugate of the invention is about 4. In an embodiment, the degree of conjugation of the glycoconjugate of the invention is about 5. In an embodiment, the degree of conjugation of the glycoconjugate of the invention is about 6. In an embodiment, the degree of conjugation of the glycoconjugate of the invention is about 8. In an embodiment, the degree of conjugation of the glycoconjugate of the invention is about 10. In an embodiment, the degree of conjugation of the glycoconjugate of the invention is about 12. In an embodiment, the degree of conjugation of the glycoconjugate of the invention is about 15. In a preferred embodiment, the degree of conjugation of the 15C glycoconjugate of the invention is between 4 and 7. In some such embodiments, the carrier protein is CRM₁₉₇.

The glycoconjugates of the invention may also be characterized by the ratio (weight/weight) of saccharide to carrier protein. In some embodiments, the ratio of polysaccharide to carrier protein in the 15C glycoconjugate (w/w) is between 0.5 and 3. In some embodiments, the ratio of polysaccharide to carrier protein in the 15C glycoconjugate (w/w) is between 0.4 and 2. In some embodiments, the ratio of polysaccharide to carrier protein in the 15C glycoconjugate (w/w) is about 0.8. In some embodiments, the ratio of polysaccharide to carrier protein in the 15C glycoconjugate (w/w) is about 0.9. In some embodiments, the ratio of polysaccharide to carrier protein in the 15C glycoconjugate (w/w) is about 1.0. In some embodiments, the ratio of polysaccharide to carrier protein in the 15C glycoconjugate (w/w) is about 1.2. In some embodiments, the ratio of polysaccharide to carrier protein in the 15C glycoconjugate (w/w) is about 1.5. In some embodiments, the ratio of polysaccharide to carrier protein in the 15C glycoconjugate (w/w) is about 1.8. In some embodiments, the ratio of polysaccharide to carrier protein in the 15C glycoconjugate (w/w) is about 2.0. In some embodiments, the ratio of polysaccharide to carrier protein in the 15C glycoconjugate (w/w) is about 2.5. In some embodiments, the ratio of polysaccharide to carrier protein in the 15C glycoconjugate (w/w) is about 3.0. In other embodiments, the saccharide to carrier protein ratio (w/w) is between 0.5 and 2.0. In other embodiments, the saccharide to carrier protein ratio (w/w) is between 0.5 and 1.5. In further embodiments, the saccharide to carrier protein ratio (w/w) is between 0.8 and 1.2. In a preferred embodiment, the ratio of capsular polysaccharide to carrier protein in the conjugate is between 0.9 and 1.1. In some such embodiments, the carrier protein is CRM₁₉₇.

The frequency of attachment of the saccharide chain to a lysine on the carrier protein is another parameter for characterizing the glycoconjugate from S. pneumoniae serotype 15C of the invention. For example, in some embodiments, at least one covalent linkage between the carrier protein and the 15C saccharide occurs for every 4 saccharide repeat units of the 15C saccharide. In another embodiment, the covalent linkage between the carrier protein and the 15C saccharide occurs at least once in every 10 saccharide repeat units of the 15C saccharide. In another embodiment, the covalent linkage between the carrier protein and the 15C saccharide occurs at least once in every 15 saccharide repeat units of the 15C saccharide. In a further embodiment, the covalent linkage between the carrier protein and the 15C saccharide occurs at least once in every 25 saccharide repeat units of the 15C saccharide.

In other embodiments, the 15C glycoconjugate comprises at least one covalent linkage between the carrier protein and 15C saccharide for every 5 to 10 saccharide repeat units. In other embodiments, the 15C glycoconjugate comprises at least one covalent linkage between the carrier protein and 15C saccharide every 2 to 7 saccharide repeat units. In other embodiments, the 15C glycoconjugate comprises at least one covalent linkage between the carrier protein and 15C saccharide for every 7 to 12 saccharide repeat units. In other embodiments, the 15C glycoconjugate comprises at least one covalent linkage between the carrier protein and 15C saccharide for every 10 to 15 saccharide repeat units. In other embodiments, the 15C glycoconjugate comprises at least one covalent linkage between the carrier protein and saccharide 15C saccharide for every 4 to 8 saccharide repeat units. In other embodiments, the 15C glycoconjugate comprises at least one covalent linkage between the carrier protein and 15C saccharide for every 10 to 20 saccharide repeat units. In other embodiments, the 15C glycoconjugate comprises at least one covalent linkage between the carrier protein and 15C saccharide for every 2 to 25 saccharide repeat units. In frequent embodiments, the carrier protein is CRM₁₉₇.

In another embodiment, at least one linkage between carrier protein and saccharide occurs for every 2, 3, 4, 5, 6, 7, 8, 9, 10, 11, 12, 13, 14, 15, 16, 17, 18, 19, 20, 21, 22, 23, 24 or 25 saccharide repeat units of the polysaccharide. In an embodiment, the carrier protein is CRM₁₉₇. Any whole number integer within any of the above ranges is contemplated as an embodiment of the disclosure.

In an embodiment, the serotype 15C glycoconjugate of the invention comprises at least 0.1 mM glycerol per mM serotype 15C capsular polysaccharide. In an embodiment, the serotype 15C glycoconjugate of the invention comprises at least 0.2 mM glycerol per mM serotype 15C capsular polysaccharide. In an embodiment, the serotype 15C glycoconjugate of the invention comprises at least 0.5 mM glycerol per mM serotype 15C capsular polysaccharide. In an embodiment, the serotype 15C glycoconjugate of the invention comprises at least 0.8 mM glycerol per mM serotype 15C capsular polysaccharide. In an embodiment, the serotype 15C glycoconjugate of the invention comprises at least 0.9 mM glycerol per mM serotype 15C capsular polysaccharide. In a preferred embodiment, the serotype 15C glycoconjugate of the invention comprises between 0.6 mM to 1 mM glycerol per mM serotype 15C capsular polysaccharide. In an even preferred embodiment, the serotype 15C glycoconjugate of the invention comprises between 0.7 mM to 1 mM glycerol per mM serotype 15C capsular polysaccharide. In a most preferred embodiment, the serotype 15C glycoconjugate of the invention comprises between 0.8 mM to 1 mM glycerol per mM serotype 15C capsular polysaccharide.

In an embodiment, the serotype 15C glycoconjugate of the invention comprises between 0.5 mM to 0.7 mM glycerol per mM serotype 15C capsular polysaccharide. In an even preferred embodiment, the serotype 15C glycoconjugate of the invention comprises about 0.7 mM glycerol per mM serotype 15C capsular polysaccharide.

In a preferred embodiment, the glycoconjugate from S. pneumoniae serotype 15C of the invention comprises less than about 50%, 45%, 40%, 35%, 30%, 25%, 20% or 15% of free polysaccharide compared to the total amount of polysaccharide. In a preferred embodiment the 15C glycoconjugate comprises less than about 25% of free polysaccharide compared to the total amount of polysaccharide. In a preferred embodiment the 15C glycoconjugate comprises less than about 20% of free polysaccharide compared to the total amount of polysaccharide. In a preferred embodiment the 15C glycoconjugate comprises less than about 15% of free polysaccharide compared to the total amount of polysaccharide.

The glycoconjugates may also be characterized by their molecular size distribution (K_{d}). Size exclusion chromatography media (CL-4B) can be used to determine the relative molecular size distribution of the conjugate. Size Exclusion Chromatography (SEC) is used in gravity fed columns to profile the molecular size distribution of conjugates. Large molecules excluded from the pores in the media elute more quickly than small molecules. Fraction collectors are used to collect the column eluate. The fractions are tested colorimetrically by saccharide assay. For the determination of K_{d}, columns are calibrated to establish the fraction at which molecules are fully excluded (V₀), (K_{d}=0), and the fraction representing the maximum retention (Vᵢ), (K_{d}=1). The fraction at which a specified sample attribute is reached (Vₑ), is related to K_{d} by the expression, K_{d}=(Vₑ−V₀)/(Vᵢ−V₀).

In a preferred embodiment, at least 30% of the glycoconjugate from S. pneumoniae serotype 15C of the invention has a K_{d} below or equal to 0.3 in a CL-4B column. In a preferred embodiment, at least 40% of the 15C glycoconjugate has a K_{d} below or equal to 0.3 in a CL-4B column. In an embodiment, at least 45%, 50%, 55%, 60%, 65%, 70%, 75%, 80%, or 85% of the 15C glycoconjugate has a K_{d} below or equal to 0.3 in a CL-4B column. In an embodiment, at least 60% of the 15C glycoconjugate has a K_{d} below or equal to 0.3 in a CL-4B column. In an embodiment, between 50% and 80% of the 15C glycoconjugate has a K_{d} below or equal to 0.3 in a CL-4B column. In a preferred embodiment, between 65% and 80% of the 15C glycoconjugate has a K_{d} below or equal to 0.3 in a CL-4B column.

1.7 Serotype 15A Glycoconjugates of the Invention

In one embodiment, the 15A glycoconjugates of the invention are as defined in the present section.

Capsular polysaccharides from serotype 15A of S. pneumoniae are prepared as disclosed above (see also WO2019/139692).

In an embodiment, the polysaccharides are activated with 1-cyano-4-dimethylamino pyridinium tetrafluoroborate (CDAP) to form a cyanate ester. The activated polysaccharide is then coupled directly or via a spacer (linker) group to an amino group on the carrier protein (preferably CRM₁₉₇). For example, the spacer could be cystamine or cysteamine to give a thiolated polysaccharide which could be coupled to the carrier via a thioether linkage obtained after reaction with a maleimide-activated carrier protein (for example using N-[γ-maleimidobutyrloxy]succinimide ester (GMBS)) or a haloacetylated carrier protein (for example using iodoacetimide, N-succinimidyl bromoacetate (SBA; SIB), N-succinimidyl(4-iodoacetyl)aminobenzoate (SIAB), sulfosuccinimidyl(4-iodoacetyl)aminobenzoate (sulfo-SIAB), N-succinimidyl iodoacetate (SIA), or succinimidyl 3-[bromoacetamido]proprionate (SBAP)). Preferably, the cyanate ester (optionally made by CDAP chemistry) is coupled with hexane diamine or adipic acid dihydrazide (ADH) and the amino-derivatised saccharide is conjugated to the carrier protein (e.g., CRM₁₉₇) using carbodiimide (e.g., EDAC or EDC) chemistry via a carboxyl group on the protein carrier. Such conjugates are described for example in WO 93/15760, WO 95/08348 and WO 96/129094.

In an embodiment of the present invention, the glycoconjugate from S. pneumoniae serotype 15A is prepared using CDAP chemistry.

Other suitable techniques for conjugation use carbodiimides, hydrazides, active esters, norborane, p-nitrobenzoic acid, N-hydroxysuccinimide, S—NHS, EDC, TSTU. Many are described in International Patent Application Publication No. WO 98/42721. Conjugation may involve a carbonyl linker which may be formed by reaction of a free hydroxyl group of the saccharide with CDI (see Bethell et al. (1979) 1. Biol. Chern. 254:2572-2574; Hearn et al. (1981) J. Chromatogr. 218:509-518) followed by reaction with a protein to form a carbamate linkage. This may involve reduction of the anomeric terminus to a primary hydroxyl group, optional protection/deprotection of the primary hydroxyl group, reaction of the primary hydroxyl group with CDI to form a CDI carbamate intermediate and coupling the CDI carbamate intermediate with an amino group on a protein.

In a preferred embodiment, capsular polysaccharide from serotype 15A of S. pneumoniae is conjugated to the carrier protein by reductive amination (such as described in U.S. Patent Appl. Pub. Nos. 2006/0228380, 2007/184072, 2007/0231340 and 2007/0184071, WO 2006/110381, WO 2008/079653, WO 2008/143709, WO2015/110942 and WO2019/139692). Therefore, in a preferred embodiment, the serotype 15A glycoconjugate of the present invention is prepared by reductive amination.

Reductive amination involves two steps as disclosed above.

In some embodiments, the glycoconjugate from S. pneumoniae serotype 15A of the present invention comprises a saccharide having a molecular weight of between 5 kDa and 2,000 kDa. In other such embodiments, the saccharide has a molecular weight of between 20 kDa and 800 kDa. In further such embodiments, the saccharide has a molecular weight of between 100 kDa to 1000 kDa. In further such embodiments, the saccharide has a molecular weight of between 100 kDa to 800 kDa. In further such embodiments, the saccharide has a molecular weight of between 100 kDa to 500 kDa. In further such embodiments, the saccharide has a molecular weight of between 100 kDa to 400 kDa. In further such embodiments, the saccharide has a molecular weight of between 100 kDa to 300 kDa. In a preferred embodiment, the saccharide has a molecular weight of between 100 kDa to 350 kDa. In another preferred embodiment, the saccharide has a molecular weight of between 100 kDa to 300 kDa. In a more preferred embodiment, the saccharide has a molecular weight of between 150 kDa to 300 kDa.

In further embodiments, the saccharide has a molecular weight of between 5 kDa to 100 kDa; 10 kDa to 100 kDa; 20 kDa to 100 kDa; 50 kDa to 100 kDa or 90 kDa to 100 kDa. Any whole number integer within any of the above ranges is contemplated as an embodiment of the disclosure. In some such embodiments, the glycoconjugate is prepared using reductive amination.

In some embodiments, the glycoconjugate from S. pneumoniae serotype 15A of the present invention has a molecular weight of between 100 kDa and 20,000 kDa. In other such embodiments, the glycoconjugate from S. pneumoniae serotype 15A of the present invention has a molecular weight of between 200 kDa and 10,000 kDa. In other such embodiments, the glycoconjugate from S. pneumoniae serotype 15A of the present invention has a molecular weight of between 2000 kDa and 5,000 kDa. In further embodiments, the glycoconjugate from S. pneumoniae serotype 15A of the invention has a molecular weight of between 1000 kDa and 5,000 kDa. In further embodiments, the glycoconjugate from S. pneumoniae serotype 15A of the invention has a molecular weight of between 1500 kDa and 4,500 kDa. In further embodiments, the glycoconjugate from S. pneumoniae serotype 15A of the invention has a molecular weight of between 2000 kDa and 4,500 kDa. In further embodiments, the glycoconjugate from S. pneumoniae serotype 15A of the invention has a molecular weight of between 500 kDa and 2,000 kDa. The molecular weight of the glycoconjugate is measured by SEC-MALLS. Any whole number integer within any of the above ranges is contemplated as an embodiment of the disclosure.

Another way to characterize the glycoconjugates of the invention is by the number of lysine residues in the carrier protein (e.g., CRM₁₉₇) that become conjugated to the saccharide which can be characterized as a range of conjugated lysines (degree of conjugation). The evidence for lysine modification of the carrier protein, due to covalent linkages to the polysaccharides, can be obtained by amino acid analysis using routine methods known to those of skill in the art. Conjugation results in a reduction in the number of lysine residues recovered, compared to the carrier protein starting material used to generate the conjugate materials. In a preferred embodiment, the degree of conjugation of the 15A glycoconjugates of the invention is between 2 and 15. In an embodiment, the degree of conjugation of the 15A glycoconjugates of the invention is between 2 and 13. In an embodiment, the degree of conjugation of the 15A glycoconjugates of the invention is between 2 and 10. In an embodiment, the degree of conjugation of the 15A glycoconjugates of the invention is between 2 and 8. In an embodiment, the degree of conjugation of the 15A glycoconjugates of the invention is between 2 and 6. In an embodiment, the degree of conjugation of the 15A glycoconjugates of the invention is between 3 and 10. In an embodiment, the degree of conjugation of the 15A glycoconjugates of the invention is between 3 and 6. In an embodiment, the degree of conjugation of the 15A glycoconjugates of the invention is between 5 and 10. In an embodiment, the degree of conjugation of the 15A glycoconjugates of the invention is between 8 and 12. In a preferred embodiment, the degree of conjugation of the 15A glycoconjugates of the invention is between 2 and 8. In an embodiment, the degree of conjugation of the glycoconjugate of the invention is about 2. In an embodiment, the degree of conjugation of the glycoconjugate of the invention is about 3. In an embodiment, the degree of conjugation of the glycoconjugate of the invention is about 4. In an embodiment, the degree of conjugation of the glycoconjugate of the invention is about 5. In an embodiment, the degree of conjugation of the glycoconjugate of the invention is about 6. In an embodiment, the degree of conjugation of the glycoconjugate of the invention is about 8. In an embodiment, the degree of conjugation of the glycoconjugate of the invention is about 10. In an embodiment, the degree of conjugation of the glycoconjugate of the invention is about 12. In an embodiment, the degree of conjugation of the glycoconjugate of the invention is about 15. In a preferred embodiment, the degree of conjugation of the 15A glycoconjugate of the invention is between 4 and 7. In some such embodiments, the carrier protein is CRM₁₉₇.

The glycoconjugates of the invention may also be characterized by the ratio (weight/weight) of saccharide to carrier protein. In some embodiments, the ratio of polysaccharide to carrier protein in the 15A glycoconjugate (w/w) is between 0.5 and 3. In some embodiments, the ratio of polysaccharide to carrier protein in the 15A glycoconjugate (w/w) is between 0.4 and 2. In some embodiments, the ratio of polysaccharide to carrier protein in the 15A glycoconjugate (w/w) is about 0.8. In some embodiments, the ratio of polysaccharide to carrier protein in the 15A glycoconjugate (w/w) is about 0.9. In some embodiments, the ratio of polysaccharide to carrier protein in the 15A glycoconjugate (w/w) is about 1.0. In some embodiments, the ratio of polysaccharide to carrier protein in the 15A glycoconjugate (w/w) is about 1.2. In some embodiments, the ratio of polysaccharide to carrier protein in the 15A glycoconjugate (w/w) is about 1.5. In some embodiments, the ratio of polysaccharide to carrier protein in the 15A glycoconjugate (w/w) is about 1.8. In some embodiments, the ratio of polysaccharide to carrier protein in the 15A glycoconjugate (w/w) is about 2.0. In some embodiments, the ratio of polysaccharide to carrier protein in the 15A glycoconjugate (w/w) is about 2.5. In some embodiments, the ratio of polysaccharide to carrier protein in the 15A glycoconjugate (w/w) is about 3.0. In other embodiments, the saccharide to carrier protein ratio (w/w) is between 0.5 and 2.0. In other embodiments, the saccharide to carrier protein ratio (w/w) is between 0.5 and 1.5. In further embodiments, the saccharide to carrier protein ratio (w/w) is between 0.8 and 1.2. In a preferred embodiment, the ratio of capsular polysaccharide to carrier protein in the conjugate is between 0.9 and 1.1. In some such embodiments, the carrier protein is CRM₁₉₇.

The frequency of attachment of the saccharide chain to a lysine on the carrier protein is another parameter for characterizing the glycoconjugate from S. pneumoniae serotype 15A of the invention. For example, in some embodiments, at least one covalent linkage between the carrier protein and the 15A saccharide occurs for every 4 saccharide repeat units of the 15A saccharide. In another embodiment, the covalent linkage between the carrier protein and the 15A saccharide occurs at least once in every 10 saccharide repeat units of the 15A saccharide. In another embodiment, the covalent linkage between the carrier protein and the 15A saccharide occurs at least once in every 15 saccharide repeat units of the 15A saccharide. In a further embodiment, the covalent linkage between the carrier protein and the 15A saccharide occurs at least once in every 25 saccharide repeat units of the 15A saccharide.

In other embodiments, the 15A glycoconjugate comprises at least one covalent linkage between the carrier protein and 15A saccharide for every 5 to 10 saccharide repeat units. In other embodiments, the 15A glycoconjugate comprises at least one covalent linkage between the carrier protein and 15A saccharide every 2 to 7 saccharide repeat units. In other embodiments, the 15A glycoconjugate comprises at least one covalent linkage between the carrier protein and 15A saccharide for every 7 to 12 saccharide repeat units. In other embodiments, the 15A glycoconjugate comprises at least one covalent linkage between the carrier protein and 15A saccharide for every 10 to 15 saccharide repeat units. In other embodiments, the 15A glycoconjugate comprises at least one covalent linkage between the carrier protein and saccharide 15A saccharide for every 4 to 8 saccharide repeat units. In other embodiments, the 15A glycoconjugate comprises at least one covalent linkage between the carrier protein and 15A saccharide for every 10 to 20 saccharide repeat units. In other embodiments, the 15A glycoconjugate comprises at least one covalent linkage between the carrier protein and 15A saccharide for every 2 to 25 saccharide repeat units. In frequent embodiments, the carrier protein is CRM₁₉₇.

In another embodiment, at least one linkage between carrier protein and saccharide occurs for every 2, 3, 4, 5, 6, 7, 8, 9, 10, 11, 12, 13, 14, 15, 16, 17, 18, 19, 20, 21, 22, 23, 24 or 25 saccharide repeat units of the polysaccharide. In an embodiment, the carrier protein is CRM₁₉₇. Any whole number integer within any of the above ranges is contemplated as an embodiment of the disclosure.

In an embodiment, the serotype 15A glycoconjugate of the invention comprises at least 0.1 mM glycerol per mM serotype 15A capsular polysaccharide. In an embodiment, the serotype 15A glycoconjugate of the invention comprises at least 0.2 mM glycerol per mM serotype 15A capsular polysaccharide. In an embodiment, the serotype 15A glycoconjugate of the invention comprises at least 0.5 mM glycerol per mM serotype 15A capsular polysaccharide. In an embodiment, the serotype 15A glycoconjugate of the invention comprises at least 0.8 mM glycerol per mM serotype 15A capsular polysaccharide. In an embodiment, the serotype 15A glycoconjugate of the invention comprises at least 0.9 mM glycerol per mM serotype 15A capsular polysaccharide. In a preferred embodiment, the serotype 15A glycoconjugate of the invention comprises between 0.6 mM to 1 mM glycerol per mM serotype 15A capsular polysaccharide. In an even preferred embodiment, the serotype 15A glycoconjugate of the invention comprises between 0.7 mM to 1 mM glycerol per mM serotype 15A capsular polysaccharide. In a most preferred embodiment, the serotype 15A glycoconjugate of the invention comprises between 0.8 mM to 1 mM glycerol per mM serotype 15A capsular polysaccharide.

In an embodiment, the serotype 15A glycoconjugate of the invention comprises between 0.5 mM to 0.7 mM glycerol per mM serotype 15A capsular polysaccharide. In an even preferred embodiment, the serotype 15A glycoconjugate of the invention comprises about 0.7 mM glycerol per mM serotype 15A capsular polysaccharide.

In a preferred embodiment, the glycoconjugate from S. pneumoniae serotype 15A of the invention comprises less than about 50%, 45%, 40%, 35%, 30%, 25%, 20% or 15% of free polysaccharide compared to the total amount of polysaccharide. In an embodiment the 15A glycoconjugate comprises less than about 25% of free polysaccharide compared to the total amount of polysaccharide. In an embodiment the 15A glycoconjugate comprises less than about 20% of free polysaccharide compared to the total amount of polysaccharide. In a preferred embodiment the 15A glycoconjugate comprises less than about 15% of free polysaccharide compared to the total amount of polysaccharide.

The glycoconjugates may also be characterized by their molecular size distribution (K_{d}). Size exclusion chromatography media (CL-4B) can be used to determine the relative molecular size distribution of the conjugate. Size Exclusion Chromatography (SEC) is used in gravity fed columns to profile the molecular size distribution of conjugates. Large molecules excluded from the pores in the media elute more quickly than small molecules. Fraction collectors are used to collect the column eluate. The fractions are tested colorimetrically by saccharide assay. For the determination of K_{d}, columns are calibrated to establish the fraction at which molecules are fully excluded (V₀), (K_{d}=0), and the fraction representing the maximum retention (Vᵢ), (K_{d}=1). The fraction at which a specified sample attribute is reached (Vₑ), is related to K_{d} by the expression, K_{d}=(Vₑ−V₀)/(Vᵢ−V₀). In an embodiment, at least 30% of the glycoconjugate from S. pneumoniae serotype 15A of the invention has a K_{d} below or equal to 0.3 in a CL-4B column. In an embodiment, at least 40% of the 15A glycoconjugate has a K_{d} below or equal to 0.3 in a CL-4B column. In a preferred embodiment, at least 45%, 50%, 55%, 60%, 65%, 70%, 75%, 80%, or 85% of the 15A glycoconjugate has a K_{d} below or equal to 0.3 in a CL-4B column. In an embodiment, at least 60% of the 15A glycoconjugate has a K_{d} below or equal to 0.3 in a CL-4B column. In an embodiment, between 50% and 80% of the 15A glycoconjugate has a K_{d} below or equal to 0.3 in a CL-4B column. In a preferred embodiment, between 65% and 80% of the 15A glycoconjugate has a K_{d} below or equal to 0.3 in a CL-4B column.

1.8 Combination of Glycoconjugates of the Invention

In an embodiment the immunogenic composition of the invention comprises a glycoconjugate from S. pneumoniae serotype 15B, a glycoconjugate from S. pneumoniae serotype 15C and a glycoconjugate from S. pneumoniae serotype 15A.

In an embodiment the immunogenic composition of the invention comprises a glycoconjugate from S. pneumoniae serotype 15B or 15C and a glycoconjugate from S. pneumoniae serotype 15A. Preferably said composition does not comprise capsular saccharide from S. pneumoniae serotype 15C when a glycoconjugate from S. pneumoniae serotype 15B is present in the composition and does not comprise capsular saccharide from S. pneumoniae serotype 15B when a glycoconjugate from S. pneumoniae serotype 15C is present.

In an embodiment the immunogenic composition of the invention comprises a glycoconjugate from S. pneumoniae serotype 15B and a glycoconjugate from S. pneumoniae serotype 15A. Preferably said composition does not comprise capsular saccharide from S. pneumoniae serotype 15C.

In an embodiment the immunogenic composition of the invention comprises a glycoconjugate from S. pneumoniae serotype 15C and a glycoconjugate from S. pneumoniae serotype 15A. Preferably said composition does not comprise capsular saccharide from S. pneumoniae serotype 15B.

In an embodiment any of the above immunogenic compositions further comprises at least one glycoconjugate from S. pneumoniae serotypes 4, 6B, 9V, 14, 18C, 19F and/or 23F.

In an embodiment any of the above immunogenic compositions further comprises at least one glycoconjugate from S. pneumoniae serotypes 4, 6B, 9V, 14, 18C, 19F and 23F.

In an embodiment any of the above immunogenic compositions further comprises at least one glycoconjugate of each of the eight following S. pneumoniae serotypes: (1, 4, 6B, 9V, 14, 18C, 19F, and 23F); (4, 5, 6B, 9V, 14, 18C, 19F, and 23F) or (4, 6B, 7F, 9V, 14, 18C, 19F, and 23F).

In an embodiment any of the above immunogenic compositions comprises at least one glycoconjugate of each of the ten following S. pneumoniae serotypes: 1, 5, 4, 6B, 7F, 9V, 14, 18C, 19F and 23F.

In an embodiment any of the above immunogenic compositions comprises at least one glycoconjugate of each of the eleven following S. pneumoniae serotypes: (1, 4, 5, 6A, 6B, 7F, 9V, 14, 18C, 19F, and 23F) or (1, 4, 5, 6B, 7F, 9V, 14, 18C, 19A, 19F, and 23F).

In an embodiment any of the above immunogenic compositions comprises at least one glycoconjugate of each of the twelve following S. pneumoniae serotypes: 1, 4, 5, 6A, 6B, 7F, 9V, 14, 18C, 19A, 19F, and 23F.

In an embodiment any of the above immunogenic compositions comprises at least one glycoconjugate of each of the thirteen following S. pneumoniae serotypes: 1, 3, 4, 5, 6A, 6B, 7F, 9V, 14, 18C, 19A, 19F, and 23F.

In an embodiment any of the above immunogenic compositions comprises in addition at least one glycoconjugate of S. pneumoniae serotype 22F.

In an embodiment any of the above immunogenic compositions above comprises in addition at least one glycoconjugate of S. pneumoniae serotype 33F.

In an embodiment any of the above immunogenic compositions above comprises in addition at least one glycoconjugate of S. pneumoniae serotype 8.

In an embodiment any of the above immunogenic compositions above comprises in addition at least one glycoconjugate of S. pneumoniae serotype 10A.

In an embodiment any of the above immunogenic compositions above comprises in addition at least one glycoconjugate of S. pneumoniae serotype 11A.

In an embodiment any of the above immunogenic compositions above comprises in addition at least one glycoconjugate of S. pneumoniae serotype 12F.

In an embodiment any of the above immunogenic compositions above comprises at least one glycoconjugate of each of the two following S. pneumoniae serotypes:
22F and 33F,
22F and 12F,
22F and 10A,
22F and 11A,
22F and 8,
33F and 12F,
33F and 10A,
33F and 11A,
33F and 8,
12F and 10A,
12F and 11A,
12F and 8,
10A and 11A,
10A and 8, or
11A and 8.

In an embodiment any of the above immunogenic compositions above comprises at least one glycoconjugate of each of the three following S. pneumoniae serotypes:
22F and 33F and 12F,
22F and 33F and 10A,
22F and 33F and 11A,
22F and 33F and 8,
22F and 12F and 10A,
22F and 12F and 11A,
22F and 12F and 8,
22F and 10A and 11A,
22F and 10A and 8,
22F and 11A and 8,
33F and 12F and 10A,
33F and 12F and 11A,
33F and 12F and 8,
33F and 10A and 11A,
33F and 10A and 8,
33F and 11A and 8,
12F and 10A and 11A,
12F and 10A and 8,
12F and 11A and 8, or
10A and 11A and 8.

In an embodiment any of the above immunogenic compositions above comprises at least one glycoconjugate of each of the four following S. pneumoniae serotypes:
22F and 33F and 12F and 10A,
22F and 33F and 12F and 11A,
22F and 33F and 12F and 8,
22F and 33F and 10A and 11A,
22F and 33F and 10A and 8,
22F and 33F and 11A and 8,
22F and 12F and 10A and 11A,
22F and 12F and 10A and 8,
22F and 12F and 11A and 8,
22F and 10A and 11A and 8,
33F and 12F and 10A and 11A,
33F and 12F and 10A and 8,
33F and 12F and 11A and 8,
33F and 10A and 11A and 8 or
12F and 10A and 11A and 8.

In an embodiment any of the above immunogenic compositions above comprises at least one glycoconjugate of each of the five following S. pneumoniae serotypes:
22F and 33F and 12F and 10A and 11A,
22F and 33F and 12F and 10A and 8,
22F and 33F and 12F and 11A and 8,
22F and 33F and 10A and 11A and 8,
22F and 12F and 10A and 11A and 8 or
33F and 12F and 10A and 11A and 8.

In an embodiment any of the above immunogenic compositions above comprises at least one glycoconjugate of each of the six following S. pneumoniae serotypes: 22F and 33F and 12F and 10A and 11A and 8.

In an embodiment any of the immunogenic composition above comprises in addition glycoconjugates from S. pneumoniae serotype 2.

In an embodiment any of the immunogenic composition above comprises in addition glycoconjugates from S. pneumoniae serotype 17F.

In an embodiment any of the immunogenic composition above comprises in addition glycoconjugates from S. pneumoniae serotype 20.

Preferably, all the glycoconjugates of the above immunogenic composition are individually conjugated to the carrier protein.

In an embodiment of any of the above immunogenic composition, the glycoconjugate from S. pneumoniae serotype 15B is conjugated to CRM₁₉₇. In an embodiment of any of the above immunogenic composition, the glycoconjugate from S. pneumoniae serotype 15C is conjugated to CRM₁₉₇. In an embodiment of any of the above immunogenic composition, the glycoconjugate from S. pneumoniae serotype 15A is conjugated to CRM₁₉₇. In an embodiment of any of the above immunogenic compositions, the glycoconjugate from S. pneumoniae serotype 22F is conjugated to CRM₁₉₇. In an embodiment of any of the above immunogenic composition, the glycoconjugate from S. pneumoniae serotype 33F is conjugated to CRM₁₉₇. In an embodiment of any of the above immunogenic composition, the glycoconjugate from S. pneumoniae serotype 12F is conjugated to CRM₁₉₇. In an embodiment of any of the above immunogenic composition, the glycoconjugate from S. pneumoniae serotype 10A is conjugated to CRM₁₉₇. In an embodiment of any of the above immunogenic composition, the glycoconjugate from S. pneumoniae serotype 11A is conjugated to CRM₁₉₇. In an embodiment of any of the above immunogenic composition, the glycoconjugate from S. pneumoniae serotype 8 is conjugated to CRM₁₉₇. In an embodiment of any of the above immunogenic composition, the glycoconjugates from S. pneumoniae serotypes 4, 6B, 9V, 14, 18C, 19F and 23F are conjugated to CRM₁₉₇. In an embodiment of any of the above immunogenic composition, the glycoconjugates from S. pneumoniae serotypes 1, 5 and 7F are conjugated to CRM₁₉₇. In an embodiment of any of the above immunogenic composition, the glycoconjugates from S. pneumoniae serotypes 6A and 19A are conjugated to CRM₁₉₇. In an embodiment of any of the above immunogenic composition, the glycoconjugates from S. pneumoniae serotype 3 is conjugated to CRM₁₉₇. In an embodiment of any of the above immunogenic compositions, the glycoconjugates from S. pneumoniae serotype 2 is conjugated to CRM₁₉₇. In an embodiment of any of the above immunogenic compositions, the glycoconjugates from S. pneumoniae serotype 17F is conjugated to CRM₁₉₇. In an embodiment of any of the above immunogenic compositions, the glycoconjugates from S. pneumoniae serotype 20 is conjugated to CRM₁₉₇. In an embodiment of any of the above immunogenic compositions, the glycoconjugates from S. pneumoniae serotype 15C is conjugated to CRM₁₉₇.

In an embodiment, the glycoconjugates of the above immunogenic compositions are all individually conjugated to CRM₁₉₇.

In an embodiment, the glycoconjugate from S. pneumoniae serotype 18C of any of the above immunogenic composition is individually conjugated to TT.

In an embodiment, the glycoconjugates from S. pneumoniae serotypes 1, 4, 5, 6B, 7F, 9V, 14 and/or 23F of any of the above immunogenic compositions are individually conjugated to PD.

In an embodiment, the glycoconjugate from S. pneumoniae serotype 19F of any of the above immunogenic compositions is conjugated to DT.

In an embodiment, the glycoconjugates from S. pneumoniae serotypes 1, 4, 5, 6B, 7F, 9V, 14 and/or 23F of any of the above immunogenic compositions are individually conjugated to PD, the glycoconjugate from S. pneumoniae serotype 18C is conjugated to TT and the glycoconjugate from S. pneumoniae serotype 19F is conjugated to DT.

In an embodiment the above immunogenic composition comprises from 2 to 25 different serotypes of S. pneumoniae. In one embodiment the above immunogenic composition comprises glycoconjugates from 2, 3, 4, 5, 6, 7, 8, 9, 10, 11, 12, 13, 14, 15, 16, 17, 18, 19, 20, 21, 22, 23, 24 or 25 different pneumococcal serotypes.

In one embodiment the above immunogenic composition comprises glycoconjugates from 2 different pneumococcal serotypes. In one embodiment the above immunogenic composition comprises glycoconjugates from 3 different pneumococcal serotypes. In one embodiment the above immunogenic composition comprises glycoconjugates from 4 different pneumococcal serotypes. In one embodiment the above immunogenic composition comprises glycoconjugates from 5 different pneumococcal serotypes. In one embodiment the above immunogenic composition comprises glycoconjugates from 6 different pneumococcal serotypes. In one embodiment the above immunogenic composition comprises glycoconjugates from 7 different pneumococcal serotypes. In one embodiment the above immunogenic composition comprises glycoconjugates from 8 different pneumococcal serotypes. In one embodiment the above immunogenic composition comprises glycoconjugates from 9 different pneumococcal serotypes. In one embodiment the above immunogenic composition comprises glycoconjugates from 10 different pneumococcal serotypes. In one embodiment the above immunogenic composition comprises glycoconjugates from 11 different pneumococcal serotypes. In one embodiment the above immunogenic composition comprises glycoconjugates from 12 different pneumococcal serotypes. In one embodiment the above immunogenic composition comprises glycoconjugates from 13 different pneumococcal serotypes. In one embodiment the above immunogenic composition comprises glycoconjugates from 14 different pneumococcal serotypes. In one embodiment the above immunogenic composition comprises glycoconjugates from 15 different pneumococcal serotypes. In one embodiment the above immunogenic composition comprises glycoconjugates from 16 different pneumococcal serotypes. In one embodiment the above immunogenic composition comprises glycoconjugates from 17 different pneumococcal serotypes. In one embodiment the above immunogenic composition comprises glycoconjugates from 18 different pneumococcal serotypes. In one embodiment the above immunogenic composition comprises glycoconjugates from 19 different pneumococcal serotypes. In one embodiment the above immunogenic composition comprises glycoconjugates from 20 different pneumococcal serotypes. In one embodiment the above immunogenic composition comprises glycoconjugates from 21 different pneumococcal serotypes. In one embodiment the above immunogenic composition comprises glycoconjugates from 22 different pneumococcal serotypes. In one embodiment the above immunogenic composition comprises glycoconjugates from 23 different pneumococcal serotypes. In one embodiment the above immunogenic composition comprises glycoconjugates from 24 different pneumococcal serotypes. In one embodiment the above immunogenic composition comprises glycoconjugates from 25 different pneumococcal serotypes. In one embodiment the above immunogenic composition comprises glycoconjugates from 16 or 20 different serotypes.

In an embodiment the above immunogenic composition is a 2, 3, 4, 5, 6, 7, 8, 9, 10, 11, 12, 13, 14, 15, 16, 17, 18, 19 or 20-valent pneumococcal conjugate composition. In an embodiment the above immunogenic composition is a 16-valent pneumococcal conjugate composition. In an embodiment the above immunogenic composition is a 19-valent pneumococcal conjugate composition. In an embodiment the above immunogenic composition is a 20-valent pneumococcal conjugate composition. In an embodiment the above immunogenic composition is a 21-valent pneumococcal conjugate composition. In an embodiment the above immunogenic composition is a 22-valent pneumococcal conjugate composition. In an embodiment the above immunogenic composition is a 2ⁿ-valent pneumococcal conjugate composition. In an embodiment the above immunogenic composition is a 23-valent pneumococcal conjugate composition. In an embodiment the above immunogenic composition is a 24-valent pneumococcal conjugate composition.

Preferably, all the glycoconjugates of the immunogenic composition of the invention are individually conjugated to the carrier protein. In an embodiment, the glycoconjugates of the immunogenic composition of the invention are individually conjugated to CRM₁₉₇. In an embodiment, the glycoconjugates of the immunogenic composition of the invention are all individually conjugated to CRM₁₉₇.

1.9 Set of Immunogenic Compositions of the Invention

An aspect of the invention pertains to a set of immunogenic compositions comprising: (a) a first immunogenic composition comprising a glycoconjugate from S. pneumoniae serotype 15C; and (b) a second immunogenic composition comprising a glycoconjugate from S. pneumoniae serotype 15A, wherein said compositions do not comprise capsular saccharide from S. pneumoniae serotypes 15B, wherein said immunogenic compositions are for simultaneous, concurrent, concomitant or sequential administration. In a preferred embodiment, said immunogenic compositions are for concurrent, concomitant or sequential administration. In a preferred embodiment, said S. pneumoniae serotype 15C is as defined at section 1.6. In a preferred embodiment, said S. pneumoniae serotype 15A is as defined at section 1.7. In an even preferred embodiment, said S. pneumoniae serotype 15C is as defined at section 1.6 and said S. pneumoniae serotype 15A is as defined at section 1.7.

In another embodiment the invention relates to a set of immunogenic compositions comprising: (a) a first immunogenic composition comprising a glycoconjugate from S. pneumoniae serotype 15B; and (b) a second immunogenic composition comprising a glycoconjugate from S. pneumoniae serotype 15A, wherein said compositions do not comprise capsular saccharide from S. pneumoniae serotypes 15C, wherein said compositions are for simultaneous, concurrent, concomitant or sequential administration.

In a preferred embodiment, said immunogenic compositions are for concurrent, concomitant or sequential administration. In a preferred embodiment, said S. pneumoniae serotype 15B is as defined at section 1.5. In a preferred embodiment, said S. pneumoniae serotype 15A is as defined at section 1.7. In an even preferred embodiment, said S. pneumoniae serotype 15B is as defined at section 1.5 and said S. pneumoniae serotype 15A is as defined at section 1.7.

In another embodiment the invention relates to a set of immunogenic compositions comprising: (a) a first immunogenic composition comprising a glycoconjugate from S. pneumoniae serotype 15B; and (b) a second immunogenic composition comprising a glycoconjugate from S. pneumoniae serotype 15A and a glycoconjugate from S. pneumoniae serotype 15C, wherein said compositions are for simultaneous, concurrent, concomitant or sequential administration. In a preferred embodiment, said immunogenic compositions are for concurrent, concomitant or sequential administration. In a preferred embodiment, said S. pneumoniae serotype 15B is as defined at section 1.5. In a preferred embodiment, said S. pneumoniae serotype 15A is as defined at section 1.7. In a preferred embodiment, said S. pneumoniae serotype 15C is as defined at section 1.6. In an even preferred embodiment, said S. pneumoniae serotype 15B is as defined at section 1.5, said S. pneumoniae serotype 15C is as defined at section 1.6 and said S. pneumoniae serotype 15A is as defined at section 1.7.

An aspect of the invention pertains to a set of immunogenic compositions consisting of: (a) a first immunogenic composition comprising a glycoconjugate from S. pneumoniae serotype 15C; and (b) a second immunogenic composition comprising a glycoconjugate from S. pneumoniae serotype 15A, wherein said compositions do not comprise capsular saccharide from S. pneumoniae serotypes 15B, wherein said immunogenic compositions are for simultaneous, concurrent, concomitant or sequential administration. In a preferred embodiment, said immunogenic compositions are for concurrent, concomitant or sequential administration. In a preferred embodiment, said S. pneumoniae serotype 15C is as defined at section 1.6. In a preferred embodiment, said S. pneumoniae serotype 15A is as defined at section 1.7. In an even preferred embodiment, said S. pneumoniae serotype 15C is as defined at section 1.6 and said S. pneumoniae serotype 15A is as defined at section 1.7. In an embodiment, said glycoconjugate from S. pneumoniae serotype 15C is conjugated to CRM₁₉₇. In an embodiment said glycoconjugate from S. pneumoniae serotype 15A is conjugated to CRM₁₉₇. In an embodiment, said glycoconjugates from S. pneumoniae serotype 15A and 15C are conjugated to CRM₁₉₇. In another embodiment the invention relates to a set of immunogenic compositions consisting of: (a) a first immunogenic composition comprising a glycoconjugate from S. pneumoniae serotype 15B; and (b) a second immunogenic composition comprising a glycoconjugate from S. pneumoniae serotype 15A, wherein said compositions do not comprise capsular saccharide from S. pneumoniae serotypes 15C, wherein said compositions are for simultaneous, concurrent, concomitant or sequential administration. In a preferred embodiment, said immunogenic compositions are for concurrent, concomitant or sequential administration. In a preferred embodiment, said S. pneumoniae serotype 15B is as defined at section 1.5. In a preferred embodiment, said S. pneumoniae serotype 15A is as defined at section 1.7. In an even preferred embodiment, said S. pneumoniae serotype 15B is as defined at section 1.5 and said S. pneumoniae serotype 15A is as defined at section 1.7. In an embodiment, said glycoconjugate from S. pneumoniae serotype 15B is conjugated to CRM₁₉₇. In an embodiment said glycoconjugate from S. pneumoniae serotype 15A is conjugated to CRM₁₉₇. In an embodiment, said glycoconjugates from S. pneumoniae serotype 15A and 15B are conjugated to CRM₁₉₇.

An aspect of the invention pertains to a set of immunogenic compositions consisting of: (a) a first immunogenic composition comprising a glycoconjugate from S. pneumoniae serotype 15B; and (b) a second immunogenic composition comprising a glycoconjugate from S. pneumoniae serotype 15A and a glycoconjugate from S. pneumoniae serotype 15C, wherein said immunogenic compositions are for simultaneous, concurrent, concomitant or sequential administration. In a preferred embodiment, said immunogenic compositions are for concurrent, concomitant or sequential administration. In a preferred embodiment, said S. pneumoniae serotype 15B is as defined at section 1.5. In a preferred embodiment, said S. pneumoniae serotype 15C is as defined at section 1.6. In a preferred embodiment, said S. pneumoniae serotype 15A is as defined at section 1.7. In an even preferred embodiment, said S. pneumoniae serotype 15B is as defined at section 1.5, said S. pneumoniae serotype 15C is as defined at section 1.6 and said S. pneumoniae serotype 15A is as defined at section 1.7. In an embodiment, said glycoconjugate from S. pneumoniae serotype 15B is conjugated to CRM₁₉₇. In an embodiment, said glycoconjugate from S. pneumoniae serotype 15C is conjugated to CRM₁₉₇. In an embodiment said glycoconjugate from S. pneumoniae serotype 15A is conjugated to CRM₁₉₇. In an embodiment, said glycoconjugates from S. pneumoniae serotype 15A, 15B and 15C are conjugated to CRM₁₉₇.

By “simultaneous administration” is meant the administration of therapeutically effective doses of a first and a second immunogenic compositions in a single unit dosage form.

By “concurrent administration” is meant the administration of therapeutically effective doses of a first and a second immunogenic compositions through the same access site, but in separate unit dosage forms, within a short period of one another. Concurrent administration is essentially administering the two immunogenic compositions at about the same time but in separate dosage forms, through the same access site. The concurrent administration of the first and the second immunogenic compositions often occurs during the same physician office visit.

By “concomitant administration” is meant the administration of therapeutically effective doses of a first and a second immunogenic compositions, in separate unit dosage forms within a short period of one another at different anatomic sites. Concomitant administration is essentially administering the two immunogenic compositions at about the same time but in separate dosage forms and at different anatomic sites. The concomitant administration of the first and second immunogenic compositions often occurs during the same physician office visit.

By “sequential administration” is meant the administration of a therapeutically effective dose of a first or a second immunogenic composition alone, followed by the administration of a therapeutically effective dose of the remaining immunogenic composition after an interval of at least about 1 month. For instance in one embodiment, the first immunogenic composition is administered in a single dosage form, and then after an interval of at least about 1 month, the second immunogenic composition is administered in a separate single dosage form. In an alternative embodiment, the second immunogenic composition is administered in a single dosage form, and then after an interval of at least about 1 month, the first immunogenic composition is administered in a separate single dosage form. The sequential administration of the first and second immunogenic compositions often occurs at different physician office visits.

1. In an embodiment said first immunogenic composition further comprises, in addition to a glycoconjugate from S. pneumoniae serotype 15B or 15C, glycoconjugates from S. pneumoniae serotypes 4, 6B, 9V, 14, 18C, 19F and 23F.

2. In another embodiment said first immunogenic composition comprises in addition to point 1 above, glycoconjugates from S. pneumoniae serotypes 1, 5 and 7F.

3. In another embodiment said first immunogenic composition comprises in addition to point 1 or 2 above, glycoconjugates from S. pneumoniae serotypes 6A and 19A.

4. In another embodiment said first immunogenic composition comprises in addition to point 1, 2 or 3 above, a glycoconjugate from S. pneumoniae serotype 3.

5. In another embodiment said first immunogenic composition comprises in addition to point 1, 2, 3 or 4 above, a glycoconjugate from S. pneumoniae serotype 22F.

6. In another embodiment said first immunogenic composition comprises in addition to point 1, 2, 3, 4 or 5 above, a glycoconjugate from S. pneumoniae serotype 33F.

In an embodiment said first immunogenic composition further comprises, in addition to a glycoconjugate from S. pneumoniae serotype 15B or 15C, glycoconjugates from S. pneumoniae serotypes 8, 10A, 11A, 12F, 22F and 33F.

Preferably, all the glycoconjugates of the above first immunogenic compositions are individually conjugated to the carrier protein.

In an embodiment of any of the above first immunogenic compositions, the glycoconjugates from S. pneumoniae serotypes 4, 6B, 9V, 14, 18C, 19F and 23F are conjugated to CRM₁₉₇. In an embodiment of any of the above first immunogenic compositions, the glycoconjugates from S. pneumoniae serotypes 1, 5 and 7F are conjugated to CRM₁₉₇. In an embodiment of any of the above first immunogenic compositions, the glycoconjugates from S. pneumoniae serotypes 6A and 19A are conjugated to CRM₁₉₇. In an embodiment of any of the above first immunogenic compositions, the glycoconjugates from S. pneumoniae serotype 3 is conjugated to CRM₁₉₇. In an embodiment of any of the above first immunogenic compositions, the glycoconjugates from S. pneumoniae serotype 22F is conjugated to CRM₁₉₇. In an embodiment of any of the above first immunogenic compositions, the glycoconjugates from S. pneumoniae serotype 33F is conjugated to CRM₁₉₇. In an embodiment of any of the above first immunogenic compositions, the glycoconjugates from S. pneumoniae serotype 10A is conjugated to CRM₁₉₇. In an embodiment of any of the above first immunogenic compositions, the glycoconjugates from S. pneumoniae serotype 11A is conjugated to CRM₁₉₇. In an embodiment of any of the above first immunogenic compositions, the glycoconjugates from S. pneumoniae serotype 12F is conjugated to CRM₁₉₇. In an embodiment of any of the above first immunogenic compositions, the glycoconjugates from S. pneumoniae serotype 8 is conjugated to CRM₁₉₇.

In an embodiment, the glycoconjugates of any of the above first immunogenic compositions are all individually conjugated to CRM₁₉₇.

In an embodiment, the glycoconjugates from S. pneumoniae serotypes 1, 4, 5, 6B, 7F, 9V, 14 and/or 23F of any of the above first immunogenic compositions are individually conjugated to PD.

In an embodiment, the glycoconjugate from S. pneumoniae serotype 18C of any of the above first immunogenic compositions is conjugated to TT.

In an embodiment, the glycoconjugate from S. pneumoniae serotype 19F of any of the above first immunogenic compositions is conjugated to DT.

In an embodiment, the glycoconjugates from S. pneumoniae serotypes 1, 4, 5, 6B, 7F, 9V, 14 and/or 23F of any of the above first immunogenic compositions are individually conjugated to PD, the glycoconjugate from S. pneumoniae serotype 18C is conjugated to TT and the glycoconjugate from S. pneumoniae serotype 19F is conjugated to DT. In an embodiment, the glycoconjugates from S. pneumoniae serotypes 1, 4, 5, 6B, 7F, 9V, 14 and/or 23F of any of the above first immunogenic compositions are individually conjugated to PD, the glycoconjugate from S. pneumoniae serotype 18C is conjugated to TT, the glycoconjugate from S. pneumoniae serotype 19F is conjugated to DT, the glycoconjugate from S. pneumoniae serotype 22F is conjugated to CRM₁₉₇ and the glycoconjugate from S. pneumoniae serotype 33F is conjugated to CRM₁₉₇.

In an embodiment the first immunogenic composition comprises from 7 to 25 different serotypes of S. pneumoniae. In an embodiment the first immunogenic composition comprises from 7 to 20 different serotypes of S. pneumoniae. In one embodiment the first immunogenic compositions comprise glycoconjugates from 7, 8, 9, 10, 11, 12, 13, 14, 15, 16, 17, 18, 19, 20, 21, 22, 23, 24 or 25 different serotypes. In one embodiment the first immunogenic compositions comprise glycoconjugates from 7 different serotypes. In one embodiment the first immunogenic compositions comprise glycoconjugates from 8 different serotypes. In one embodiment the first immunogenic compositions comprise glycoconjugates from 10 different serotypes. In one embodiment the first immunogenic compositions comprise glycoconjugates from 15 different serotypes. In one embodiment the first immunogenic compositions comprise glycoconjugates from 16 different serotypes. In one embodiment the first immunogenic compositions comprise glycoconjugates from 20 different serotypes. In an embodiment the first immunogenic composition is a 7, 8, 9, 10, 11, 12, 13, 14, 15, 16, 17, 18, 19, 20, 21, 22, 23, 24 or 25-valent pneumococcal conjugate composition. In an embodiment the first immunogenic composition is a 7-valent pneumococcal conjugate composition. In an embodiment the first immunogenic composition is an 8-valent pneumococcal conjugate composition. In an embodiment the first immunogenic composition is a 9-valent pneumococcal conjugate composition. In an embodiment the first immunogenic composition is a 10-valent pneumococcal conjugate composition. In an embodiment the above first immunogenic composition is an 11-valent pneumococcal conjugate composition. In an embodiment the above first immunogenic composition is a 12-valent pneumococcal conjugate composition. In an embodiment the above first immunogenic composition is a 13-valent pneumococcal conjugate composition. In an embodiment the above first immunogenic composition is a 14-valent pneumococcal conjugate composition. In an embodiment the above first immunogenic composition is a 15-valent pneumococcal conjugate composition. In an embodiment the first immunogenic composition is a 16-valent pneumococcal conjugate composition. In an embodiment the first immunogenic composition is a 17-valent pneumococcal conjugate composition. In an embodiment the first immunogenic composition is an 18-valent pneumococcal conjugate composition. In an embodiment the first immunogenic composition is a 19-valent pneumococcal conjugate composition. In an embodiment the first immunogenic composition is a 20-valent pneumococcal conjugate composition. In an embodiment the first immunogenic composition is a 21-valent pneumococcal conjugate composition. In an embodiment the first immunogenic composition is a 22-valent pneumococcal conjugate composition. In an embodiment the first immunogenic composition is a 23-valent pneumococcal conjugate composition. In an embodiment the first immunogenic composition is a 24-valent pneumococcal conjugate composition. In an embodiment the first immunogenic composition is a 25-valent pneumococcal conjugate composition.

In an embodiment, the first immunogenic composition is an 8-valent pneumococcal conjugate composition wherein in addition to a glycoconjugate from S. pneumoniae serotype 15B or 15C, said composition further comprises, glycoconjugates from S. pneumoniae serotypes 4, 6B, 9V, 14, 18C, 19F and 23F individually conjugated to CRM₁₉₇.

In an embodiment, the above first immunogenic composition is a 14-valent pneumococcal conjugate composition wherein in addition to a glycoconjugate from S. pneumoniae serotype 15B or 15C, said composition further comprises, glycoconjugates from S. pneumoniae serotypes 1, 3, 4, 5, 6A, 6B, 7F, 9V, 14, 18C, 19A, 19F and 23F individually conjugated to CRM₁₉₇.

In an embodiment, the above first immunogenic composition is a 15-valent pneumococcal conjugate composition wherein in addition to a glycoconjugate from S. pneumoniae serotype 15B or 15C, said composition further comprises, glycoconjugates from S. pneumoniae serotypes 1, 3, 4, 5, 6A, 6B, 7F, 9V, 14, 18C, 19A, 19F, 23F and 22F individually conjugated to CRM₁₉₇.

In an embodiment, the above first immunogenic composition is a 15-valent pneumococcal conjugate composition wherein in addition to a glycoconjugate from S. pneumoniae serotype 15B or 15C, said composition further comprises, glycoconjugates from S. pneumoniae serotypes 1, 3, 4, 5, 6A, 6B, 7F, 9V, 14, 18C, 19A, 19F, 23F and 33F individually conjugated to CRM₁₉₇.

In an embodiment, the above first immunogenic composition is a 16-valent pneumococcal conjugate composition wherein in addition to a glycoconjugate from S. pneumoniae serotype 15B or 15C, said composition further comprises, glycoconjugates from S. pneumoniae serotypes 1, 3, 4, 5, 6A, 6B, 7F, 9V, 14, 18C, 19A, 19F, 23F, 22F and 33F individually conjugated to CRM₁₉₇.

In an embodiment, the above first immunogenic composition is a 20-valent pneumococcal conjugate composition wherein in addition to a glycoconjugate from S. pneumoniae serotype 15B or 15C, said composition further comprises, glycoconjugates from S. pneumoniae serotypes 1, 3, 4, 5, 6A, 6B, 7F, 8, 9V, 10A, 11A, 12F, 14, 18C, 19A, 19F, 23F, 22F and 33F individually conjugated to CRM₁₉₇.

In an embodiment the first immunogenic compositions further comprise one or more adjuvants as disclosed at section 4 below.

In an embodiment the above first immunogenic compositions are formulated as disclosed at section 5 below.

1. In an embodiment said second immunogenic composition further comprises, in addition to a glycoconjugate from S. pneumoniae serotype 15A, glycoconjugates from S. pneumoniae serotypes 4, 6B, 9V, 14, 18C, 19F and 23F.

2. In another embodiment said second immunogenic composition comprises in addition to point 1 above, glycoconjugates from S. pneumoniae serotypes 1, 5 and 7F.

3. In another embodiment said second immunogenic composition comprises in addition to point 1 or 2 above, glycoconjugates from S. pneumoniae serotypes 6A and 19A.

4. In another embodiment said second immunogenic composition comprises in addition to point 1, 2 or 3 above, a glycoconjugate from S. pneumoniae serotype 3.

5. In another embodiment said second immunogenic composition comprises in addition to point 1, 2, 3 or 4 above, a glycoconjugate from S. pneumoniae serotype 22F.

6. In another embodiment said second immunogenic composition comprises in addition to point 1, 2, 3, 4 or 5 above, a glycoconjugate from S. pneumoniae serotype 33F.

Preferably, all the glycoconjugates of the above second immunogenic compositions are individually conjugated to the carrier protein.

In an embodiment of any of the above second immunogenic compositions, the glycoconjugates from S. pneumoniae serotypes 4, 6B, 9V, 14, 18C, 19F and 23F are conjugated to CRM₁₉₇. In an embodiment of any of the above second immunogenic compositions, the glycoconjugates from S. pneumoniae serotypes 1, 5 and 7F are conjugated to CRM₁₉₇. In an embodiment of any of the above second immunogenic compositions, the glycoconjugates from S. pneumoniae serotypes 6A and 19A are conjugated to CRM₁₉₇. In an embodiment of any of the above second immunogenic compositions, the glycoconjugates from S. pneumoniae serotype 3 is conjugated to CRM₁₉₇. In an embodiment of any of the above second immunogenic compositions, the glycoconjugates from S. pneumoniae serotype 22F is conjugated to CRM₁₉₇. In an embodiment of any of the above second immunogenic compositions, the glycoconjugates from S. pneumoniae serotype 33F is conjugated to CRM₁₉₇. In an embodiment of any of the above second immunogenic compositions, the glycoconjugates from S. pneumoniae serotype 10A is conjugated to CRM₁₉₇. In an embodiment of any of the above second immunogenic compositions, the glycoconjugates from S. pneumoniae serotype 11A is conjugated to CRM₁₉₇. In an embodiment of any of the above second immunogenic compositions, the glycoconjugates from S. pneumoniae serotype 12F is conjugated to CRM₁₉₇. In an embodiment of any of the above second immunogenic compositions, the glycoconjugates from S. pneumoniae serotype 8 is conjugated to CRM₁₉₇.

In an embodiment, the glycoconjugates of any of the above second immunogenic compositions are all individually conjugated to CRM₁₉₇.

In an embodiment, the glycoconjugates from S. pneumoniae serotypes 1, 4, 5, 6B, 7F, 9V, 14 and/or 23F of any of the above second immunogenic compositions are individually conjugated to PD.

In an embodiment, the glycoconjugate from S. pneumoniae serotype 18C of any of the above second immunogenic compositions is conjugated to TT.

In an embodiment, the glycoconjugate from S. pneumoniae serotype 19F of any of the above second immunogenic compositions is conjugated to DT.

In an embodiment, the glycoconjugates from S. pneumoniae serotypes 1, 4, 5, 6B, 7F, 9V, 14 and/or 23F of any of the above second immunogenic compositions are individually conjugated to PD, the glycoconjugate from S. pneumoniae serotype 18C is conjugated to TT and the glycoconjugate from S. pneumoniae serotype 19F is conjugated to DT.

In an embodiment, the glycoconjugates from S. pneumoniae serotypes 1, 4, 5, 6B, 7F, 9V, 14 and/or 23F of any of the above second immunogenic compositions are individually conjugated to PD, the glycoconjugate from S. pneumoniae serotype 18C is conjugated to TT, the glycoconjugate from S. pneumoniae serotype 19F is conjugated to DT, the glycoconjugate from S. pneumoniae serotype 22F is conjugated to CRM₁₉₇ and the glycoconjugate from S. pneumoniae serotype 33F is conjugated to CRM₁₉₇.

In an embodiment the second immunogenic composition comprises from 7 to 25 different serotypes of S. pneumoniae. In an embodiment the second immunogenic composition comprises from 7 to 20 different serotypes of S. pneumoniae. In one embodiment the second immunogenic compositions comprise glycoconjugates from 7, 8, 9, 10, 11, 12, 13, 14, 15, 16, 17, 18, 19, 20, 21, 22, 23, 24 or 25 different serotypes. In one embodiment the second immunogenic compositions comprise glycoconjugates from 7 different serotypes. In one embodiment the second immunogenic compositions comprise glycoconjugates from 8 different serotypes. In one embodiment the second immunogenic compositions comprise glycoconjugates from 10 different serotypes. In one embodiment the second immunogenic compositions comprise glycoconjugates from 15 different serotypes. In one embodiment the second immunogenic compositions comprise glycoconjugates from 16 different serotypes. In one embodiment the second immunogenic compositions comprise glycoconjugates from 20 different serotypes. In an embodiment the second immunogenic composition is a 7, 8, 9, 10, 11, 12, 13, 14, 15, 16, 17, 18, 19, 20, 21, 22, 23, 24 or 25-valent pneumococcal conjugate composition. In an embodiment the second immunogenic composition is a 7-valent pneumococcal conjugate composition. In an embodiment the second immunogenic composition is an 8-valent pneumococcal conjugate composition. In an embodiment the second immunogenic composition is a 9-valent pneumococcal conjugate composition. In an embodiment the second immunogenic composition is a 10-valent pneumococcal conjugate composition. In an embodiment the above second immunogenic composition is an 11-valent pneumococcal conjugate composition. In an embodiment the above second immunogenic composition is a 12-valent pneumococcal conjugate composition. In an embodiment the above second immunogenic composition is a 13-valent pneumococcal conjugate composition. In an embodiment the above second immunogenic composition is a 14-valent pneumococcal conjugate composition. In an embodiment the above second immunogenic composition is a 15-valent pneumococcal conjugate composition. In an embodiment the second immunogenic composition is a 16-valent pneumococcal conjugate composition. In an embodiment the second immunogenic composition is a 17-valent pneumococcal conjugate composition. In an embodiment the second immunogenic composition is an 18-valent pneumococcal conjugate composition. In an embodiment the second immunogenic composition is a 19-valent pneumococcal conjugate composition. In an embodiment the second immunogenic composition is a 20-valent pneumococcal conjugate composition. In an embodiment the second immunogenic composition is a 21-valent pneumococcal conjugate composition. In an embodiment the second immunogenic composition is a 22-valent pneumococcal conjugate composition. In an embodiment the second immunogenic composition is a 23-valent pneumococcal conjugate composition. In an embodiment the second immunogenic composition is a 24-valent pneumococcal conjugate composition. In an embodiment the second immunogenic composition is a 25-valent pneumococcal conjugate composition.

In an embodiment, the second immunogenic composition is an 8-valent pneumococcal conjugate composition wherein in addition to a glycoconjugate from S. pneumoniae serotype 15A, said composition further comprises, glycoconjugates from S. pneumoniae serotypes 4, 68, 9V, 14, 18C, 19F and 23F individually conjugated to CRM₁₉₇.

In an embodiment, the above second immunogenic composition is a 14-valent pneumococcal conjugate composition wherein in addition to a glycoconjugate from S. pneumoniae serotype 15A, said composition further comprises, glycoconjugates from S. pneumoniae serotypes 1, 3, 4, 5, 6A, 6B, 7F, 9V, 14, 18C, 19A, 19F and 23F individually conjugated to CRM₁₉₇.

In an embodiment, the above second immunogenic composition is a 15-valent pneumococcal conjugate composition wherein in addition to a glycoconjugate from S. pneumoniae serotype 15A, said composition further comprises, glycoconjugates from S. pneumoniae serotypes 1, 3, 4, 5, 6A, 6B, 7F, 9V, 14, 18C, 19A, 19F, 23F and 22F individually conjugated to CRM₁₉₇.

In an embodiment, the above second immunogenic composition is a 15-valent pneumococcal conjugate composition wherein in addition to a glycoconjugate from S. pneumoniae serotype 15A, said composition further comprises, glycoconjugates from S. pneumoniae serotypes 1, 3, 4, 5, 6A, 6B, 7F, 9V, 14, 18C, 19A, 19F, 23F and 33F individually conjugated to CRM₁₉₇.

In an embodiment, the above second immunogenic composition is a 16-valent pneumococcal conjugate composition wherein in addition to a glycoconjugate from S. pneumoniae serotype 15A, said composition further comprises, glycoconjugates from S. pneumoniae serotypes 1, 3, 4, 5, 6A, 6B, 7F, 9V, 14, 18C, 19A, 19F, 23F, 22F and 33F individually conjugated to CRM₁₉₇.

In an embodiment, the above second immunogenic composition is a 20-valent pneumococcal conjugate composition wherein in addition to a glycoconjugate from S. pneumoniae serotype 15A, said composition further comprises, glycoconjugates from S. pneumoniae serotypes 1, 3, 4, 5, 6A, 6B, 7F, 8, 9V, 10A, 11A, 12F, 14, 18C, 19A, 19F, 23F, 22F and 33F individually conjugated to CRM₁₉₇.

In an embodiment, the second immunogenic composition is a 9-valent pneumococcal conjugate composition wherein in addition to a glycoconjugate from S. pneumoniae serotype 15A and a glycoconjugate from S. pneumoniae serotype 15C, said composition further comprises, glycoconjugates from S. pneumoniae serotypes 4, 68, 9V, 14, 18C, 19F and 23F individually conjugated to CRM₁₉₇.

In an embodiment, the above second immunogenic composition is a 15-valent pneumococcal conjugate composition wherein in addition to a glycoconjugate from S. pneumoniae serotype 15A and a glycoconjugate from S. pneumoniae serotype 15C, said composition further comprises, glycoconjugates from S. pneumoniae serotypes 1, 3, 4, 5, 6A, 6B, 7F, 9V, 14, 18C, 19A, 19F and 23F individually conjugated to CRM₁₉₇.

In an embodiment, the above second immunogenic composition is a 16-valent pneumococcal conjugate composition wherein in addition to a glycoconjugate from S. pneumoniae serotype 15A and a glycoconjugate from S. pneumoniae serotype 15C, said composition further comprises, glycoconjugates from S. pneumoniae serotypes 1, 3, 4, 5, 6A, 6B, 7F, 9V, 14, 18C, 19A, 19F, 23F and 22F individually conjugated to CRM₁₉₇.

In an embodiment, the above second immunogenic composition is a 16-valent pneumococcal conjugate composition wherein in addition to a glycoconjugate from S. pneumoniae serotype 15A and a glycoconjugate from S. pneumoniae serotype 15C, said composition further comprises, glycoconjugates from S. pneumoniae serotypes 1, 3, 4, 5, 6A, 6B, 7F, 9V, 14, 18C, 19A, 19F, 23F and 33F individually conjugated to CRM₁₉₇.

In an embodiment, the above second immunogenic composition is a 17-valent pneumococcal conjugate composition wherein in addition to a glycoconjugate from S. pneumoniae serotype 15A and a glycoconjugate from S. pneumoniae serotype 15C, said composition further comprises, glycoconjugates from S. pneumoniae serotypes 1, 3, 4, 5, 6A, 6B, 7F, 9V, 14, 18C, 19A, 19F, 23F, 22F and 33F individually conjugated to CRM₁₉₇.

In an embodiment, the above second immunogenic composition is a 21-valent pneumococcal conjugate composition wherein in addition to a glycoconjugate from S. pneumoniae serotype 15A and a glycoconjugate from S. pneumoniae serotype 15C, said composition further comprises, glycoconjugates from S. pneumoniae serotypes 1, 3, 4, 5, 6A, 6B, 7F, 8, 9V, 10A, 11A, 12F, 14, 18C, 19A, 19F, 23F, 22F and 33F individually conjugated to CRM₁₉₇.

In an embodiment the second immunogenic compositions further comprise one or more adjuvants as disclosed at section 4 below.

In an embodiment the above second immunogenic compositions are formulated as disclosed at section 5 below.

1.10 Complementary Vaccine to Achieve Optimal Protection Against S. pneumoniae Serotypes 15A, 15B and 15C of the Invention

In an embodiment the immunogenic composition of the invention comprises a glycoconjugate from S. pneumoniae serotype 15A but does not comprise glycoconjugates from S. pneumoniae serotypes 15B and 15C.

In an embodiment the immunogenic composition of the invention comprises a glycoconjugate from S. pneumoniae serotype 15B but does not comprise glycoconjugates from S. pneumoniae serotypes 15A and 15C.

In an embodiment the immunogenic composition of the invention comprises a glycoconjugate from S. pneumoniae serotype 15C but does not comprise glycoconjugates from S. pneumoniae serotypes 15A and 15B.

In an embodiment the immunogenic composition of the invention comprises a glycoconjugate from S. pneumoniae serotype 15A and a glycoconjugate from S. pneumoniae serotype 15C but does not comprise a glycoconjugate from S. pneumoniae serotype 15B.

In an embodiment the immunogenic composition of the invention comprises a glycoconjugate from S. pneumoniae serotype 15A and a glycoconjugate from S. pneumoniae serotype 15B but does not comprise a glycoconjugate from S. pneumoniae serotype 15C.

In an embodiment the immunogenic composition of the invention comprises a glycoconjugate from S. pneumoniae serotype 15B and a glycoconjugate from S. pneumoniae serotype 15C but does not comprise a glycoconjugate from S. pneumoniae serotype 15A.

In an embodiment the immunogenic composition of the invention comprises a glycoconjugate from S. pneumoniae serotype 15A but does not comprise glycoconjugates from S. pneumoniae serotypes 4, 6B, 9V, 14, 15B, 15C, 18C, 19F and 23F.

In an embodiment the immunogenic composition of the invention comprises a glycoconjugate from S. pneumoniae serotype 15B but does not comprise glycoconjugates from S. pneumoniae serotypes 4, 6B, 9V, 14, 15A, 15C, 18C, 19F and 23F.

In an embodiment the immunogenic composition of the invention comprises a glycoconjugate from S. pneumoniae serotype 15C but does not comprise glycoconjugates from S. pneumoniae serotypes 4, 6B, 9V, 14, 15A, 15B, 18C, 19F and 23F.

In an embodiment the immunogenic composition of the invention comprises a glycoconjugate from S. pneumoniae serotype 15A and a glycoconjugate from S. pneumoniae serotype 15C but does not comprise glycoconjugates from S. pneumoniae serotypes 4, 68, 9V, 14, 15B, 18C, 19F and 23F.

In an embodiment the immunogenic composition of the invention comprises a glycoconjugate from S. pneumoniae serotype 15A and a glycoconjugate from S. pneumoniae serotype 15B but does not comprise glycoconjugates from S. pneumoniae serotypes 4, 6B, 9V, 14, 15C, 18C, 19F and 23F.

In an embodiment the immunogenic composition of the invention comprises a glycoconjugate from S. pneumoniae serotype 15B and a glycoconjugate from S. pneumoniae serotype 15C but does not comprise glycoconjugates from S. pneumoniae serotypes 4, 68, 9V, 14, 15A, 18C, 19F and 23F.

In an embodiment the immunogenic composition of the invention comprises a glycoconjugate from S. pneumoniae serotype 15A but does not comprise glycoconjugates from S. pneumoniae serotypes 1, 4, 5, 6B, 7F, 9V, 14, 15B, 15C, 18C, 19F and 23F.

In an embodiment the immunogenic composition of the invention comprises a glycoconjugate from S. pneumoniae serotype 15B but does not comprise glycoconjugates from S. pneumoniae serotypes 1, 4, 5, 6B, 7F, 9V, 14, 15A, 15C, 18C, 19F and 23F.

In an embodiment the immunogenic composition of the invention comprises a glycoconjugate from S. pneumoniae serotype 15C but does not comprise glycoconjugates from S. pneumoniae serotypes 1, 4, 5, 6B, 7F, 9V, 14, 15A, 15B, 18C, 19F and 23F.

In an embodiment the immunogenic composition of the invention comprises a glycoconjugate from S. pneumoniae serotype 15A and a glycoconjugate from S. pneumoniae serotype 15C but does not comprise glycoconjugates from S. pneumoniae serotypes 1, 4, 5, 6B, 7F, 9V, 14, 15B, 18C, 19F and 23F.

In an embodiment the immunogenic composition of the invention comprises a glycoconjugate from S. pneumoniae serotype 15A and a glycoconjugate from S. pneumoniae serotype 15B but does not comprise glycoconjugates from S. pneumoniae serotypes 1, 4, 5, 6B, 7F, 9V, 14, 15C, 18C, 19F and 23F.

In an embodiment the immunogenic composition of the invention comprises a glycoconjugate from S. pneumoniae serotype 15B and a glycoconjugate from S. pneumoniae serotype 15C but does not comprise glycoconjugates from S. pneumoniae serotypes 1, 4, 5, 6B, 7F, 9V, 14, 15A, 18C, 19F and 23F.

In an embodiment the immunogenic composition of the invention comprises a glycoconjugate from S. pneumoniae serotype 15A but does not comprise glycoconjugates from S. pneumoniae serotypes 1, 4, 5, 6A, 6B, 7F, 9V, 14, 15B, 15C, 18C, 19A, 19F and 23F.

In an embodiment the immunogenic composition of the invention comprises a glycoconjugate from S. pneumoniae serotype 15B but does not comprise glycoconjugates from S. pneumoniae serotypes 1, 4, 5, 6A, 6B, 7F, 9V, 14, 15A, 15C, 18C, 19A, 19F and 23F.

In an embodiment the immunogenic composition of the invention comprises a glycoconjugate from S. pneumoniae serotype 15C but does not comprise glycoconjugates from S. pneumoniae serotypes 1, 4, 5, 6A, 6B, 7F, 9V, 14, 15A, 15B, 18C, 19A, 19F and 23F.

In an embodiment the immunogenic composition of the invention comprises a glycoconjugate from S. pneumoniae serotype 15A and a glycoconjugate from S. pneumoniae serotype 15C but does not comprise glycoconjugates from S. pneumoniae serotypes 1, 4, 5, 6A, 6B, 7F, 9V, 14, 15B, 18C, 19A, 19F and 23F.

In an embodiment the immunogenic composition of the invention comprises a glycoconjugate from S. pneumoniae serotype 15A and a glycoconjugate from S. pneumoniae serotype 15B but does not comprise glycoconjugates from S. pneumoniae serotypes 1, 4, 5, 6A, 6B, 7F, 9V, 14, 15C, 18C, 19A, 19F and 23F.

In an embodiment the immunogenic composition of the invention comprises a glycoconjugate from S. pneumoniae serotype 15B and a glycoconjugate from S. pneumoniae serotype 15C but does not comprise glycoconjugates from S. pneumoniae serotypes 1, 4, 5, 6A, 6B, 7F, 9V, 14, 15A, 18C, 19A, 19F and 23F.

In an embodiment the immunogenic composition of the invention comprises a glycoconjugate from S. pneumoniae serotype 15A but does not comprise glycoconjugates from S. pneumoniae serotypes 1, 3, 4, 5, 6A, 6B, 7F, 9V, 14, 15B, 15C, 18C, 19A, 19F and 23F.

In an embodiment the immunogenic composition of the invention comprises a glycoconjugate from S. pneumoniae serotype 15B but does not comprise glycoconjugates from S. pneumoniae serotypes 1, 3, 4, 5, 6A, 6B, 7F, 9V, 14, 15A, 15C, 18C, 19A, 19F and 23F.

In an embodiment the immunogenic composition of the invention comprises a glycoconjugate from S. pneumoniae serotype 15C but does not comprise glycoconjugates from S. pneumoniae serotypes 1, 3, 4, 5, 6A, 6B, 7F, 9V, 14, 15A, 15B, 18C, 19A, 19F and 23F.

In an embodiment the immunogenic composition of the invention comprises a glycoconjugate from S. pneumoniae serotype 15A and a glycoconjugate from S. pneumoniae serotype 15C but does not comprise glycoconjugates from S. pneumoniae serotypes 1, 3, 4, 5, 6A, 6B, 7F, 9V, 14, 15B, 18C, 19A, 19F and 23F.

In an embodiment the immunogenic composition of the invention comprises a glycoconjugate from S. pneumoniae serotype 15A and a glycoconjugate from S. pneumoniae serotype 15B but does not comprise glycoconjugates from S. pneumoniae serotypes 1, 3, 4, 5, 6A, 6B, 7F, 9V, 14, 15C, 18C, 19A, 19F and 23F.

In an embodiment the immunogenic composition of the invention comprises a glycoconjugate from S. pneumoniae serotype 15B and a glycoconjugate from S. pneumoniae serotype 15C but does not comprise glycoconjugates from S. pneumoniae serotypes 1, 3, 4, 5, 6A, 6B, 7F, 9V, 14, 15A, 18C, 19A, 19F and 23F.

In an embodiment the immunogenic composition of the invention comprises a glycoconjugate from S. pneumoniae serotype 15A but does not comprise glycoconjugates from S. pneumoniae serotypes 1, 4, 5, 6A, 6B, 7F, 9V, 14, 15B, 15C, 18C, 19A, 19F, 22F, 23F and 33F.

In an embodiment the immunogenic composition of the invention comprises a glycoconjugate from S. pneumoniae serotype 15B but does not comprise glycoconjugates from S. pneumoniae serotypes 1, 4, 5, 6A, 6B, 7F, 9V, 14, 15A, 15C, 18C, 19A, 19F, 22F, 23F and 33F.

In an embodiment the immunogenic composition of the invention comprises a glycoconjugate from S. pneumoniae serotype 15C but does not comprise glycoconjugates from S. pneumoniae serotypes 1, 4, 5, 6A, 6B, 7F, 9V, 14, 15A, 15B, 18C, 19A, 19F, 22F, 23F and 33F.

In an embodiment the immunogenic composition of the invention comprises a glycoconjugate from S. pneumoniae serotype 15A and a glycoconjugate from S. pneumoniae serotype 15C but does not comprise glycoconjugates from S. pneumoniae serotypes 1, 4, 5, 6A, 6B, 7F, 9V, 14, 15B, 18C, 19A, 19F, 22F, 23F and 33F.

In an embodiment the immunogenic composition of the invention comprises a glycoconjugate from S. pneumoniae serotype 15A and a glycoconjugate from S. pneumoniae serotype 15B but does not comprise glycoconjugates from S. pneumoniae serotypes 1, 4, 5, 6A, 6B, 7F, 9V, 14, 15C, 18C, 19A, 19F, 22F, 23F and 33F.

In an embodiment the immunogenic composition of the invention comprises a glycoconjugate from S. pneumoniae serotype 15B and a glycoconjugate from S. pneumoniae serotype 15C but does not comprise glycoconjugates from S. pneumoniae serotypes 1, 4, 5, 6A, 6B, 7F, 9V, 14, 15A, 18C, 19A, 19F, 22F, 23F and 33F.

In an embodiment the immunogenic composition of the invention comprises a glycoconjugate from S. pneumoniae serotype 15A but does not comprise glycoconjugates from S. pneumoniae serotypes 1, 3, 4, 5, 6A, 6B, 7F, 9V, 14, 15B, 15C, 18C, 19A, 19F, 22F, 23F and 33F.

In an embodiment the immunogenic composition of the invention comprises a glycoconjugate from S. pneumoniae serotype 15B but does not comprise glycoconjugates from S. pneumoniae serotypes 1, 3, 4, 5, 6A, 6B, 7F, 9V, 14, 15A, 15C, 18C, 19A, 19F, 22F, 23F and 33F.

In an embodiment the immunogenic composition of the invention comprises a glycoconjugate from S. pneumoniae serotype 15C but does not comprise glycoconjugates from S. pneumoniae serotypes 1, 3, 4, 5, 6A, 6B, 7F, 9V, 14, 15A, 15B, 18C, 19A, 19F, 22F, 23F and 33F.

In an embodiment the immunogenic composition of the invention comprises a glycoconjugate from S. pneumoniae serotype 15A and a glycoconjugate from S. pneumoniae serotype 15C but does not comprise glycoconjugates from S. pneumoniae serotypes 1, 3, 4, 5, 6A, 6B, 7F, 9V, 14, 15B, 18C, 19A, 19F, 22F, 23F and 33F.

In an embodiment the immunogenic composition of the invention comprises a glycoconjugate from S. pneumoniae serotype 15A and a glycoconjugate from S. pneumoniae serotype 15B but does not comprise glycoconjugates from S. pneumoniae serotypes 1, 3, 4, 5, 6A, 6B, 7F, 9V, 14, 15C, 18C, 19A, 19F, 22F, 23F and 33F.

In an embodiment the immunogenic composition of the invention comprises a glycoconjugate from S. pneumoniae serotype 15B and a glycoconjugate from S. pneumoniae serotype 15C but does not comprise glycoconjugates from S. pneumoniae serotypes 1, 3, 4, 5, 6A, 6B, 7F, 9V, 14, 15A, 18C, 19A, 19F, 22F, 23F and 33F.

In an embodiment the immunogenic composition of the invention comprises a glycoconjugate from S. pneumoniae serotype 15A but does not comprise glycoconjugates from S. pneumoniae serotypes 1, 4, 5, 6A, 6B, 7F, 8, 9V, 10A, 11A, 12F, 14, 15B, 15C, 18C, 19A, 19F, 22F, 23F and 33F.

In an embodiment the immunogenic composition of the invention comprises a glycoconjugate from S. pneumoniae serotype 15B but does not comprise glycoconjugates from S. pneumoniae serotypes 1, 4, 5, 6A, 6B, 7F, 8, 9V, 10A, 11A, 12F, 14, 15A, 15C, 18C, 19A, 19F, 22F, 23F and 33F.

In an embodiment the immunogenic composition of the invention comprises a glycoconjugate from S. pneumoniae serotype 15C but does not comprise glycoconjugates from S. pneumoniae serotypes 1, 4, 5, 6A, 6B, 7F, 8, 9V, 10A, 11A, 12F, 14, 15A, 15B, 18C, 19A, 19F, 22F, 23F and 33F.

In an embodiment the immunogenic composition of the invention comprises a glycoconjugate from S. pneumoniae serotype 15A and a glycoconjugate from S. pneumoniae serotype 15C but does not comprise glycoconjugates from S. pneumoniae serotypes 1, 4, 5, 6A, 6B, 7F, 8, 9V, 10A, 11A, 12F, 14, 15B, 18C, 19A, 19F, 22F, 23F and 33F In an embodiment the immunogenic composition of the invention comprises a glycoconjugate from S. pneumoniae serotype 15A and a glycoconjugate from S. pneumoniae serotype 15B but does not comprise glycoconjugates from S. pneumoniae serotypes 1, 4, 5, 6A, 6B, 7F, 8, 9V, 10A, 11A, 12F, 14, 15C, 18C, 19A, 19F, 22F, 23F and 33F.

In an embodiment the immunogenic composition of the invention comprises a glycoconjugate from S. pneumoniae serotype 15B and a glycoconjugate from S. pneumoniae serotype 15C but does not comprise glycoconjugates from S. pneumoniae serotypes 1, 4, 5, 6A, 6B, 7F, 8, 9V, 10A, 11A, 12F, 14, 15A, 18C, 19A, 19F, 22F, 23F and 33F.

In an embodiment the immunogenic composition of the invention comprises a glycoconjugate from S. pneumoniae serotype 15A but does not comprise glycoconjugates from S. pneumoniae serotypes 1, 4, 5, 6A, 6B, 7F, 8, 9V, 10A, 11A, 12F, 14, 15B, 18C, 19A, 19F, 22F, 23F and 33F.

In an embodiment the immunogenic composition of the invention comprises a glycoconjugate from S. pneumoniae serotype 15C but does not comprise glycoconjugates from S. pneumoniae serotypes 1, 4, 5, 6A, 6B, 7F, 8, 9V, 10A, 11A, 12F, 14, 15B, 18C, 19A, 19F, 22F, 23F and 33F.

In an embodiment the immunogenic composition of the invention comprises a glycoconjugate from S. pneumoniae serotype 15A but does not comprise glycoconjugates from S. pneumoniae serotypes 1, 3, 4, 5, 6A, 6B, 7F, 8, 9V, 10A, 11A, 12F, 14, 15B, 15C, 18C, 19A, 19F, 22F, 23F and 33F.

In an embodiment the immunogenic composition of the invention comprises a glycoconjugate from S. pneumoniae serotype 15B but does not comprise glycoconjugates from S. pneumoniae serotypes 1, 3, 4, 5, 6A, 6B, 7F, 8, 9V, 10A, 11A, 12F, 14, 15A, 15C, 18C, 19A, 19F, 22F, 23F and 33F.

In an embodiment the immunogenic composition of the invention comprises a glycoconjugate from S. pneumoniae serotype 15C but does not comprise glycoconjugates from S. pneumoniae serotypes 1, 3, 4, 5, 6A, 6B, 7F, 8, 9V, 10A, 11A, 12F, 14, 15A, 15B, 18C, 19A, 19F, 22F, 23F and 33F.

In an embodiment the immunogenic composition of the invention comprises a glycoconjugate from S. pneumoniae serotype 15A and a glycoconjugate from S. pneumoniae serotype 15C but does not comprise glycoconjugates from S. pneumoniae serotypes 1, 3, 4, 5, 6A, 6B, 7F, 8, 9V, 10A, 11A, 12F, 14, 15B, 18C, 19A, 19F, 22F, 23F and 33F In an embodiment the immunogenic composition of the invention comprises a glycoconjugate from S. pneumoniae serotype 15A and a glycoconjugate from S. pneumoniae serotype 15B but does not comprise glycoconjugates from S. pneumoniae serotypes 1, 3, 4, 5, 6A, 6B, 7F, 8, 9V, 10A, 11A, 12F, 14, 15C, 18C, 19A, 19F, 22F, 23F and 33F.

In an embodiment the immunogenic composition of the invention comprises a glycoconjugate from S. pneumoniae serotype 15B and a glycoconjugate from S. pneumoniae serotype 15C but does not comprise glycoconjugates from S. pneumoniae serotypes 1, 3, 4, 5, 6A, 6B, 7F, 8, 9V, 10A, 11A, 12F, 14, 15A, 18C, 19A, 19F, 22F, 23F and 33F.

In an embodiment the immunogenic composition of the invention comprises a glycoconjugate from S. pneumoniae serotype 15A but does not comprise glycoconjugates from S. pneumoniae serotypes 1, 3, 4, 5, 6A, 6B, 7F, 8, 9V, 10A, 11A, 12F, 14, 15B, 18C, 19A, 19F, 22F, 23F and 33F.

In an embodiment the immunogenic composition of the invention comprises a glycoconjugate from S. pneumoniae serotype 15C but does not comprise glycoconjugates from S. pneumoniae serotypes 1, 3, 4, 5, 6A, 6B, 7F, 8, 9V, 10A, 11A, 12F, 14, 15B, 18C, 19A, 19F, 22F, 23F and 33F.

Preferably, all the glycoconjugates of the above immunogenic composition are individually conjugated to the carrier protein.

In an embodiment of any of the above immunogenic composition, the glycoconjugate from S. pneumoniae serotype 15C is conjugated to CRM₁₉₇. In an embodiment of any of the above immunogenic composition, the glycoconjugates from S. pneumoniae serotype 15B is conjugated to CRM₁₉₇. In an embodiment of any of the above immunogenic composition, the glycoconjugates from S. pneumoniae serotype 15A is conjugated to CRM₁₉₇.

In an embodiment the above immunogenic composition comprises glycoconjugates from 2 to 25 different serotypes of S. pneumoniae. In one embodiment the above immunogenic composition comprises glycoconjugates from 2, 3, 4, 5, 6, 7, 8, 9, 10, 11, 12, 13, 14, 15, 16, 17, 18, 19, 20, 21, 22, 23, 24 or 25 different pneumococcal serotypes.

In one embodiment the above immunogenic composition comprises glycoconjugates from 2 different pneumococcal serotypes. In one embodiment the above immunogenic composition comprises glycoconjugates from 3 different pneumococcal serotypes. In one embodiment the above immunogenic composition comprises glycoconjugates from 4 different pneumococcal serotypes. In one embodiment the above immunogenic composition comprises glycoconjugates from 5 different pneumococcal serotypes. In one embodiment the above immunogenic composition comprises glycoconjugates from 6 different pneumococcal serotypes. In one embodiment the above immunogenic composition comprises glycoconjugates from 7 different pneumococcal serotypes. In one embodiment the above immunogenic composition comprises glycoconjugates from 8 different pneumococcal serotypes. In one embodiment the above immunogenic composition comprises glycoconjugates from 9 different pneumococcal serotypes. In one embodiment the above immunogenic composition comprises glycoconjugates from 10 different pneumococcal serotypes. In one embodiment the above immunogenic composition comprises glycoconjugates from 11 different pneumococcal serotypes. In one embodiment the above immunogenic composition comprises glycoconjugates from 12 different pneumococcal serotypes. In one embodiment the above immunogenic composition comprises glycoconjugates from 13 different pneumococcal serotypes. In one embodiment the above immunogenic composition comprises glycoconjugates from 14 different pneumococcal serotypes. In one embodiment the above immunogenic composition comprises glycoconjugates from 15 different pneumococcal serotypes. In one embodiment the above immunogenic composition comprises glycoconjugates from 16 different pneumococcal serotypes. In one embodiment the above immunogenic composition comprises glycoconjugates from 17 different pneumococcal serotypes. In one embodiment the above immunogenic composition comprises glycoconjugates from 18 different pneumococcal serotypes. In one embodiment the above immunogenic composition comprises glycoconjugates from 19 different pneumococcal serotypes. In one embodiment the above immunogenic composition comprises glycoconjugates from 20 different pneumococcal serotypes. In one embodiment the above immunogenic composition comprises glycoconjugates from 21 different pneumococcal serotypes. In one embodiment the above immunogenic composition comprises glycoconjugates from 22 different pneumococcal serotypes. In one embodiment the above immunogenic composition comprises glycoconjugates from 23 different pneumococcal serotypes. In one embodiment the above immunogenic composition comprises glycoconjugates from 24 different pneumococcal serotypes. In one embodiment the above immunogenic composition comprises glycoconjugates from 25 different pneumococcal serotypes. In one embodiment the above immunogenic composition comprises glycoconjugates from 16 or 20 different serotypes.

In an embodiment the above immunogenic composition is a 2, 3, 4, 5, 6, 7, 8, 9, 10, 11, 12, 13, 14, 15, 16, 17, 18, 19 or 20-valent pneumococcal conjugate composition. In an embodiment the above immunogenic composition is a 16-valent pneumococcal conjugate composition. In an embodiment the above immunogenic composition is a 19-valent pneumococcal conjugate composition. In an embodiment the above immunogenic composition is a 20-valent pneumococcal conjugate composition. In an embodiment the above immunogenic composition is a 21-valent pneumococcal conjugate composition. Preferably, all the glycoconjugates of the immunogenic composition of the invention are individually conjugated to the carrier protein. In an embodiment, the glycoconjugates of the immunogenic composition of the invention are individually conjugated to CRM₁₉₇. In an embodiment, the glycoconjugates of the immunogenic composition of the invention are all individually conjugated to CRM₁₉₇.

The immunogenic compositions of the present section are also particularly useful to complement vaccines of the prior art to achieve optimal protection against S. pneumoniae serotypes 15A, 15B and 15C.

In particular, the immunogenic compositions of the present section are particularly useful to complement vaccines of the prior art such as Prevnar®, Prevnar13® or Synflorix® to achieve optimal protection against S. pneumoniae serotypes 15A, 15B and 15C.

In an embodiment, the immunogenic compositions of the present section are for use to complement a 10-valent pneumococcal conjugate vaccine wherein said conjugates consist of glycoconjugates from S. pneumoniae serotypes 1, 4, 5, 6B, 7F, 9V, 14, 18C, 19F and 23F. In an embodiment, said glycoconjugates from S. pneumoniae serotypes 1, 4, 5, 6B, 7F, 9V, 14 and 23F are conjugated to PD, the glycoconjugate from S. pneumoniae serotype 18C is conjugated to TT and the glycoconjugate from S. pneumoniae serotype 19F is conjugated to DT.

In an embodiment, the immunogenic compositions of the present section are for use to complement a vaccine which comprises glycoconjugates from S. pneumoniae serotypes 1, 4, 5, 6A, 6B, 7F, 9V, 14, 18C, 19A, 19F and 23F.

In an embodiment, the immunogenic compositions of the present section are for use to complement a 12-valent pneumococcal conjugate vaccine wherein said conjugates consist of glycoconjugates from S. pneumoniae serotypes 1, 4, 5, 6A, 6B, 7F, 9V, 14, 18C, 19A, 19F and 23F. In an embodiment, said glycoconjugates from S. pneumoniae serotypes 1, 4, 5, 6A, 6B, 7F, 9V, 14, 18C, 19A, 19F and 23F are conjugated to CRM₁₉₇.

In an embodiment, the immunogenic compositions of the present section are for use to complement a vaccine which comprises glycoconjugates from S. pneumoniae serotypes 1, 3, 4, 5, 6A, 6B, 7F, 9V, 14, 18C, 19A, 19F and 23F.

In an embodiment, the immunogenic compositions of the present section are for use to complement a 13-valent pneumococcal conjugate vaccine wherein said conjugates consist of glycoconjugates from S. pneumoniae serotypes 1, 3, 4, 5, 6A, 6B, 7F, 9V, 14, 18C, 19A, 19F and 23F. In an embodiment, said glycoconjugates from S. pneumoniae serotypes 1, 3, 4, 5, 6A, 6B, 7F, 9V, 14, 18C, 19A, 19F and 23F are conjugated to CRM₁₉₇.

In an embodiment, the immunogenic compositions of the present section are for use to complement a vaccine which comprises glycoconjugates from S. pneumoniae serotypes 1, 4, 5, 6A, 6B, 7F, 9V, 14, 18C, 19A, 19F, 22F, 23F and 33F.

In an embodiment, the immunogenic compositions of the present section are for use to complement a 14-valent pneumococcal conjugate vaccine wherein said conjugates consist of glycoconjugates from S. pneumoniae serotypes 1, 4, 5, 6A, 6B, 7F, 9V, 14, 18C, 19A, 19F, 22F, 23F and 33F. In an embodiment, said glycoconjugates from S. pneumoniae serotypes 1, 4, 5, 6A, 6B, 7F, 9V, 14, 18C, 19A, 19F, 22F, 23F and 33F are conjugated to CRM₁₉₇.

In an embodiment, the immunogenic compositions of the present section are for use to complement a vaccine which comprises glycoconjugates from S. pneumoniae serotypes 1, 3, 4, 5, 6A, 6B, 7F, 9V, 14, 18C, 19A, 19F, 22F, 23F and 33F.

In an embodiment, the immunogenic compositions of the present section are for use to complement a 15-valent pneumococcal conjugate vaccine wherein said conjugates consist of glycoconjugates from S. pneumoniae serotypes 1, 3, 4, 5, 6A, 6B, 7F, 9V, 14, 18C, 19A, 19F, 22F, 23F and 33F. In an embodiment, said glycoconjugates from S. pneumoniae serotypes 1, 3, 4, 5, 6A, 6B, 7F, 9V, 14, 18C, 19A, 19F, 22F, 23F and 33F are conjugated to CRM₁₉₇.

In an embodiment, the immunogenic compositions of the present section are for use to complement a vaccine which comprises glycoconjugates from S. pneumoniae serotypes 1, 4, 5, 6A, 6B, 7F, 8, 9V, 10A, 11A, 12F, 14, 15B, 18C, 19A, 19F, 22F, 23F and 33F.

In an embodiment, the immunogenic compositions of the present section are for use to complement a 19-valent pneumococcal conjugate vaccine wherein said conjugates consist of glycoconjugates from S. pneumoniae serotypes 1, 4, 5, 6A, 6B, 7F, 8, 9V, 10A, 11A, 12F, 14, 15B, 18C, 19A, 19F, 22F, 23F and 33F. In an embodiment, said glycoconjugates from S. pneumoniae serotypes 1, 4, 5, 6A, 6B, 7F, 8, 9V, 10A, 11A, 12F, 14, 15B, 18C, 19A, 19F, 22F, 23F and 33F are conjugated to CRM₁₉₇.

In an embodiment, the immunogenic compositions of the present section are for use to complement a vaccine which comprises glycoconjugates from S. pneumoniae serotypes 1, 3, 4, 5, 6A, 6B, 7F, 8, 9V, 10A, 11A, 12F, 14, 15B, 18C, 19A, 19F, 22F, 23F and 33F.

In an embodiment, the immunogenic compositions of the present section are for use to complement a 20-valent pneumococcal conjugate vaccine wherein said conjugates consist of glycoconjugates from S. pneumoniae serotypes 1, 3, 4, 5, 6A, 6B, 7F, 8, 9V, 10A, 11A, 12F, 14, 15B, 18C, 19A, 19F, 22F, 23F and 33F. In an embodiment, said glycoconjugates from S. pneumoniae serotypes 1, 3, 4, 5, 6A, 6B, 7F, 8, 9V, 10A, 11A, 12F, 14, 15B, 18C, 19A, 19F, 22F, 23F and 33F are conjugated to CRM₁₉₇.

In an embodiment, the immunogenic compositions of the present section are for use to complement a 22-valent pneumococcal conjugate vaccine wherein said conjugates consist of glycoconjugates from S. pneumoniae serotypes 1, 3, 4, 5, 6A, 6B, 7F, 9V, 10A, 12F, 14, 15A, 15C, 18C, 19A, 19F, 22F, 23B, 23F, 24F, 33F and 35B. In an embodiment, said glycoconjugates from S. pneumoniae serotypes 1, 3, 4, 5, 6A, 6B, 7F, 9V, 10A, 12F, 14, 15A, 15C, 18C, 19A, 19F, 22F, 23B, 23F, 24F, 33F and 35B are conjugated to CRM₁₉₇.

In an embodiment, the immunogenic compositions of the present section are for use to complement a 23-valent pneumococcal conjugate vaccine wherein said conjugates consist of glycoconjugates from S. pneumoniae serotypes 1, 3, 4, 5, 6A, 6B, 7F, 8, 9V, 10A, 12F, 14, 15A, 15C, 18C, 19A, 19F, 22F, 23B, 23F, 24F, 33F and 35B. In an embodiment, said glycoconjugates from S. pneumoniae serotypes 1, 3, 4, 5, 6A, 6B, 7F, 8, 9V, 10A, 12F, 14, 15A, 15C, 18C, 19A, 19F, 22F, 23B, 23F, 24F, 33F and 35B are conjugated to CRM₁₉₇.

In an embodiment, the immunogenic compositions of the present section are for use to complement a 24-valent pneumococcal conjugate vaccine wherein said conjugates consist of glycoconjugates from S. pneumoniae serotypes 1, 3, 4, 5, 6A, 6B, 7F, 8, 9V, 10A, 11A, 12F, 14, 15A, 15C, 18C, 19A, 19F, 22F, 23B, 23F, 24F, 33F and 35B. In an embodiment, said glycoconjugates from S. pneumoniae serotypes 1, 3, 4, 5, 6A, 6B, 7F, 8, 9V, 10A, 11A, 12F, 14, 15A, 15C, 18C, 19A, 19F, 22F, 23B, 23F, 24F, 33F and 35B are conjugated to CRM₁₉₇.

By “use to complement” or “complementing” a particular vaccine is meant the use of an immunogenic composition to broaden the protection of said vaccine against S. pneumoniae serotype(s) for which said vaccine does not provide significant protection. In particular, the immunogenic compositions of the present section are useful to complement a vaccine to achieve significant protection against S. pneumoniae serotypes 15A, 15B and/or 15C, preferably against S. pneumoniae serotypes 15A, 15B and 15C.

Therefore, in an embodiment, the immunogenic compositions of the present section which comprise glycoconjugates from S. pneumoniae serotypes 15B and 15C are for use to complement a pneumococcal conjugate vaccine which comprises a glycoconjugate from S. pneumoniae serotype 15A but which does not comprise glycoconjugates from S. pneumoniae serotypes 15B and 15C.

In an embodiment, the immunogenic compositions of the present section which comprise glycoconjugates from S. pneumoniae serotypes 15A and 15C are for use to complement a pneumococcal conjugate vaccine which comprises a glycoconjugate from S. pneumoniae serotype 15B but which does not comprise glycoconjugates from S. pneumoniae serotypes 15A and 15C.

In an embodiment, the immunogenic compositions of the present section which comprise glycoconjugates from S. pneumoniae serotypes 15A and 15B are for use to complement a pneumococcal conjugate vaccine which comprises a glycoconjugate from S. pneumoniae serotype 15C but which does not comprise glycoconjugates from S. pneumoniae serotypes 15A and 15B.

In an embodiment, the immunogenic compositions of the present section which comprise a glycoconjugate from S. pneumoniae serotype 15C are for use to complement a pneumococcal conjugate vaccine which comprises a glycoconjugate from S. pneumoniae serotype 15A and a glycoconjugate from S. pneumoniae serotype 15B but which does not comprise a glycoconjugate from S. pneumoniae serotypes 15C.

In an embodiment, the immunogenic compositions of the present section which comprise a glycoconjugate from S. pneumoniae serotype 15C are for use to complement a pneumococcal conjugate vaccine which comprises a glycoconjugate from S. pneumoniae serotype 15A but which does not comprise a glycoconjugate from S. pneumoniae serotypes 15C.

In an embodiment, the immunogenic compositions of the present section which comprise a glycoconjugate from S. pneumoniae serotype 15C are for use to complement a pneumococcal conjugate vaccine which comprises a glycoconjugate from S. pneumoniae serotype 15B but which does not comprise a glycoconjugate from S. pneumoniae serotypes 15C.

In an embodiment, the immunogenic compositions of the present section which comprise a glycoconjugate from S. pneumoniae serotype 15B are for use to complement a pneumococcal conjugate vaccine which comprises a glycoconjugate from S. pneumoniae serotype 15A and a glycoconjugate from S. pneumoniae serotype 15C but which does not comprise a glycoconjugate from S. pneumoniae serotypes 15B.

In an embodiment, the immunogenic compositions of the present section which comprise a glycoconjugate from S. pneumoniae serotype 15B are for use to complement a pneumococcal conjugate vaccine which comprises a glycoconjugate from S. pneumoniae serotype 15A but which does not comprise a glycoconjugate from S. pneumoniae serotypes 15B.

In an embodiment, the immunogenic compositions of the present section which comprise a glycoconjugate from S. pneumoniae serotype 15B are for use to complement a pneumococcal conjugate vaccine which comprises a glycoconjugate from S. pneumoniae serotype 15C but which does not comprise a glycoconjugate from S. pneumoniae serotypes 15B.

In an embodiment, the immunogenic compositions of the present section which comprise a glycoconjugate from S. pneumoniae serotype 15A are for use to complement a pneumococcal conjugate vaccine which comprises a glycoconjugate from S. pneumoniae serotype 15B and a glycoconjugate from S. pneumoniae serotype 15C but which does not comprise a glycoconjugate from S. pneumoniae serotypes 15A.

In an embodiment, the immunogenic compositions of the present section which comprise a glycoconjugate from S. pneumoniae serotype 15A are for use to complement a pneumococcal conjugate vaccine which comprises a glycoconjugate from S. pneumoniae serotype 15B but which does not comprise a glycoconjugate from S. pneumoniae serotypes 15A.

In an embodiment, the immunogenic compositions of the present section which comprise a glycoconjugate from S. pneumoniae serotype 15A are for use to complement a pneumococcal conjugate vaccine which comprises a glycoconjugate from S. pneumoniae serotype 15C but which does not comprise a glycoconjugate from S. pneumoniae serotypes 15A.

2 Dosage of the Immunogenic Compositions

2.1 Polysaccharide Amount

The amount of glycoconjugate(s) in each dose is selected as an amount which induces an immunoprotective response without significant, adverse side effects in typical vaccinees. Such amount will vary depending upon which specific immunogen is employed and how it is presented.

The amount of a particular glycoconjugate in an immunogenic composition can be calculated based on total polysaccharide for that conjugate (conjugated and non-conjugated). For example, a glycoconjugate with 20% free polysaccharide will have about 80 μg of conjugated polysaccharide and about 20 μg of non-conjugated polysaccharide in a 100 μg polysaccharide dose. The amount of glycoconjugate can vary depending upon the streptococcal serotype. The saccharide concentration can be determined by the uronic acid assay.

The “immunogenic amount” of the different polysaccharide components in the immunogenic composition, may diverge and each may comprise about 1.0 μg, about 2.0 μg, about 3.0 μg, about 4.0 μg, about 5.0 μg, about 6.0 μg, about 7.0 μg, about 8.0 μg, about 9.0 μg, about 10.0 μg, about 15.0 μg, about 20.0 μg, about 30.0 μg, about 40.0 μg, about 50.0 μg, about 60.0 μg, about 70.0 μg, about 80.0 μg, about 90.0 μg, or about 100.0 μg of any particular polysaccharide antigen.

Generally, each dose will comprise 0.1 μg to 100 μg of polysaccharide for a given serotype, particularly 0.5 μg to 20 μg, more particularly 1 μg to 10 μg, and even more particularly 2 μg to 5 μg. Any whole number integer within any of the above ranges is contemplated as an embodiment of the disclosure.

In an embodiment, each dose will comprise 1 μg, 2 μg, 3 μg, 4 μg, 5 μg, 6 μg, 7 μg, 8 μg, 9 μg, 10 μg, 15 μg or 20 μg of polysaccharide for a given serotype.

2.2 Carrier Amount

Generally, each dose will comprise 5 μg to 150 μg of carrier protein, particularly 10 μg to 100 μg of carrier protein, more particularly 15 μg to 100 μg of carrier protein, more particularly 25 to 75 μg of carrier protein, more particularly 30 μg to 70 μg of carrier protein, more particularly 30 to 60 μg of carrier protein, more particularly 30 μg to 50 μg of carrier protein and even more particularly 40 to 60 μg of carrier protein. In an embodiment, said carrier protein is CRM₁₉₇.

In an embodiment, each dose will comprise about 25 μg, about 26 μg, about 27 μg, about 28 μg, about 29 μg, about 30 μg, about 31 μg, about 32 μg, about 33 μg, about 34 μg, about 35 μg, about 36 μg, about 37 μg, about 38 μg, about 39 μg, about 40 μg, about 41 μg, about 42 μg, about 43 μg, about 44 μg, about 45 μg, about 46 μg, about 47 μg, about 48 μg, about 49 μg, about 50 μg, about 51 μg, about 52 μg, about 53 μg, about 54 μg, about 55 μg, about 56 μg, about 57 μg, about 58 μg, about 59 μg, about 60 μg, about 61 μg, about 62 μg, about 63 μg, about 64 μg, about 65 μg, about 66 μg, about 67 μg, 68 μg, about 69 μg, about 70 μg, about 71 μg, about 72 μg, about 73 μg, about 74 μg or about 75 μg of carrier protein. In an embodiment, said carrier protein is CRM₁₉₇.

3 Further Antigens

Immunogenic compositions of the invention comprise conjugated S. pneumoniae saccharide antigens (glycoconjugates). They may also further include antigens from other pathogens, particularly from bacteria and/or viruses. Preferred further antigens are selected from: a diphtheria toxoid (D), a tetanus toxoid (T), a pertussis antigen (P), which is typically acellular (Pa), a hepatitis B virus (HBV) surface antigen (HBsAg), a hepatitis A virus (HAV) antigen, a conjugated Haemophilus influenzae type b capsular saccharide (Hib), inactivated poliovirus vaccine (IPV).

In an embodiment, the immunogenic compositions of the invention comprise D-T-Pa. In an embodiment, the immunogenic compositions of the invention comprise D-T-Pa-Hib, D-T-Pa-IPV or D-T-Pa-HBsAg. In an embodiment, the immunogenic compositions of the invention comprise D-T-Pa-HBsAg-IPV or D-T-Pa-HBsAg-Hib. In an embodiment, the immunogenic compositions of the invention comprise D-T-Pa-HBsAg-IPV-Hib.

4 Adjuvant(s) In some embodiments, the immunogenic compositions disclosed herein may further comprise at least one adjuvant (e.g., one, two or three adjuvants). The term “adjuvant” refers to a compound or mixture that enhances the immune response to an antigen. Antigens may act primarily as a delivery system, primarily as an immune modulator or have strong features of both. Suitable adjuvants include those suitable for use in mammals, including humans.

Examples of known suitable delivery-system type adjuvants that can be used in humans include, but are not limited to, alum (e.g., aluminum phosphate, aluminum sulfate or aluminum hydroxide), calcium phosphate, liposomes, oil-in-water emulsions such as MF59 (4.3% w/v squalene, 0.5% w/v polysorbate 80 (Tween 80), 0.5% w/v sorbitan trioleate (Span 85)), water-in-oil emulsions such as Montanide, and poly(D,L-lactide-co-glycolide) (PLG) microparticles or nanoparticles.

In an embodiment, the immunogenic compositions disclosed herein comprise aluminum salts (alum) as adjuvant (e.g., aluminum phosphate, aluminum sulfate or aluminum hydroxide). In a preferred embodiment, the immunogenic compositions disclosed herein comprise aluminum phosphate or aluminum hydroxide as adjuvant. In an embodiment, the immunogenic compositions disclosed herein comprise from 0.1 mg/mL to 1 mg/mL or from 0.2 mg/mL to 0.3 mg/ml of elemental aluminum in the form of aluminum phosphate. In an embodiment, the immunogenic compositions disclosed herein comprise about 0.25 mg/mL of elemental aluminum in the form of aluminum phosphate.

Examples of known suitable immune modulatory type adjuvants that can be used in humans include, but are not limited to, saponin extracts from the bark of the Aquilla tree (QS21, Quil A), TLR4 agonists such as MPL (Monophosphoryl Lipid A), 3DMPL (3-O-deacylated MPL) or GLA-AQ, LT/CT mutants, cytokines such as the various interleukins (e.g., IL-2, IL-12) or GM-CSF, and the like.

Examples of known suitable immune modulatory type adjuvants with both delivery and immune modulatory features that can be used in humans include, but are not limited to ISCOMS (see, e.g., Sjölander et al. (1998) J. Leukocyte Biol. 64:713; WO 90/03184, WO 96/11711, WO 00/48630, WO 98/36772, WO 00/41720, WO 2006/134423 and WO 2007/026190) or GLA-EM which is a combination of a TLR4 agonist and an oil-in-water emulsion.

For veterinary applications including but not limited to animal experimentation, one can use Complete Freund's Adjuvant (CFA), Freund's Incomplete Adjuvant (IFA), Emulsigen, N-acetyl-muramyl-L-threonyl-D-isoglutamine (thr-MDP), N-acetyl-nor-muramyl-L-alanyl-D-isoglutamine (CGP 11637, referred to as nor-MDP), N-acetylmuramyl-L-alanyl-D-isoglutaminyl-L-alanine-2-(1′-2′-dipalmitoyl-sn-glycero-3-hydroxyphosphoryloxy)-ethylamine (CGP 19835A, referred to as MTP-PE), and RIBI, which contains three components extracted from bacteria, monophosphoryl lipid A, trehalose dimycolate and cell wall skeleton (MPL+TDM+CWS) in a 2% squalene/Tween 80 emulsion.

Further exemplary adjuvants to enhance effectiveness of the pneumococcal vaccines as disclosed herein include, but are not limited to: (1) oil-in-water emulsion formulations (with or without other specific immunostimulating agents such as muramyl peptides (see below) or bacterial cell wall components), such as for example (a) SAF, containing 10% Squalane, 0.4% Tween 80, 5% pluronic-blocked polymer L121, and thr-MDP either microfluidized into a submicron emulsion or vortexed to generate a larger particle size emulsion, and (b) RIBI™ adjuvant system (RAS), (Ribi Immunochem, Hamilton, MT) containing 2% Squalene, 0.2% Tween 80, and one or more bacterial cell wall components such as monophosphorylipid A (MPL), trehalose dimycolate (TDM), and cell wall skeleton (CWS), preferably MPL+CWS (DETOX™); (2) saponin adjuvants, such as QS21, STIMULON™ (Cambridge Bioscience, Worcester, MA), Abisco® (Isconova, Sweden), or Iscomatrix® (Commonwealth Serum Laboratories, Australia), may be used or particles generated therefrom such as ISCOMs (immunostimulating complexes), which ISCOMS may be devoid of additional detergent (e.g., WO 00/07621); (3) Complete Freund's Adjuvant (CFA) and Incomplete Freund's Adjuvant (IFA); (4) cytokines, such as interleukins (e.g., IL-1, IL-2, IL-4, IL-5, IL-6, IL-7, IL-12 (WO 99/44636)), interferons (e.g., gamma interferon), macrophage colony stimulating factor (M-CSF), tumor necrosis factor (TNF), etc.; (5) monophosphoryl lipid A (MPL) or 3-O-deacylated MPL (3dMPL) (see, e.g., GB-2220221, EP0689454), optionally in the substantial absence of alum when used with pneumococcal saccharides (see, e.g., WO 00/56358); (6) combinations of 3dMPL with, for example, QS21 and/or oil-in-water emulsions (see, e.g., EP0835318, EP0735898, EP0761231); (7) a polyoxyethylene ether or a polyoxyethylene ester (see, e.g., WO99/52549); (8) a polyoxyethylene sorbitan ester surfactant in combination with an octoxynol (WO 01/21207) or a polyoxyethylene alkyl ether or ester surfactant in combination with at least one additional non-ionic surfactant such as an octoxynol (WO 01/21152); (9) a saponin and an immunostimulatory oligonucleotide (e.g., a CpG oligonucleotide) (WO 00/62800); (10) an immunostimulant and a particle of metal salt (see e.g., WO00/23105); (11) a saponin and an oil-in-water emulsion e.g., WO 99/11241; (12) a saponin (e.g., QS21)+3dMPL+IM2 (optionally+a sterol) e.g., WO 98/57659; (13) other substances that act as immunostimulating agents to enhance the efficacy of the composition. Muramyl peptides include N-acetyl-muramyl-L-threonyl-D-isoglutamine (thr-MDP), N-25 acetyl-normuramyl-L-alanyl-D-isoglutamine (nor-MDP), N-acetylmuramyl-L-alanyl-D-isoglutarninyl-L-alanine-2-(1′-2′-dipalmitoyl-sn-glycero-3-hydroxyphosphoryloxy)-ethylamine MTP-PE), etc.

In an embodiment of the present invention, the immunogenic compositions as disclosed herein comprise a CpG Oligonucleotide as adjuvant. A CpG oligonucleotide as used herein refers to an immunostimulatory CpG oligodeoxynucleotide (CpG ODN), and accordingly these terms are used interchangeably unless otherwise indicated. Immunostimulatory CpG oligodeoxynucleotides contain one or more immunostimulatory CpG motifs that are unmethylated cytosine-guanine dinucleotides, optionally within certain preferred base contexts. The methylation status of the CpG immunostimulatory motif generally refers to the cytosine residue in the dinucleotide. An immunostimulatory oligonucleotide containing at least one unmethylated CpG dinucleotide is an oligonucleotide which contains a 5′ unmethylated cytosine linked by a phosphate bond to a 3′ guanine, and which activates the immune system through binding to Toll-like receptor 9 (TLR-9). In another embodiment the immunostimulatory oligonucleotide may contain one or more methylated CpG dinucleotides, which will activate the immune system through TLR9 but not as strongly as if the CpG motif(s) was/were unmethylated. CpG immunostimulatory oligonucleotides may comprise one or more palindromes that in turn may encompass the CpG dinucleotide. CpG oligonucleotides have been described in a number of issued patents, published patent applications, and other publications, including U.S. Pat. Nos. 6,194,388; 6,207,646; 6,214,806; 6,218,371; 6,239,116; and 6,339,068. In an embodiment of the present invention, the immunogenic compositions as disclosed herein comprise any of the CpG Oligonucleotide described at pages 3, lines 22, to page 12, line 36, of WO 2010/125480.

Different classes of CpG immunostimulatory oligonucleotides have been identified. These are referred to as A, B, C and P class, and are described in greater detail at pages 3, lines 22, to page 12, line 36, of WO 2010/125480. Methods of the invention embrace the use of these different classes of CpG immunostimulatory oligonucleotides.

In an embodiment of the present invention, the immunogenic compositions as disclosed herein comprise an A class CpG oligonucleotide. In an embodiment of the present invention, the immunogenic compositions as disclosed herein comprise a B class CpG Oligonucleotide.

The B class CpG oligonucleotide sequences of the invention are those broadly described above as well as disclosed in published WO 96/02555, WO 98/18810, and in U.S. Pat. Nos. 6,194,388; 6,207,646; 6,214,806; 6,218,371; 6,239,116; and 6,339,068. Exemplary sequences include but are not limited to those disclosed in these latter applications and patents.

In an embodiment, the “B class” CpG oligonucleotide of the invention has the following nucleic acid sequence:

(SEQ ID NO: 1)
5′ TCGTCGTTTTTCGGTGCTTTT 3′,
or
(SEQ ID NO: 2)
5′ TCGTCGTTTTTCGGTCGTTTT 3′,
or
(SEQ ID NO: 3)
5′ TCGTCGTTTTGTCGTTTTGTCGTT 3′,
or
(SEQ ID NO: 4)
5′ TCGTCGTTTCGTCGTTTTGTCGTT 3′,
or
(SEQ ID NO: 5)
5′ TCGTCGTTTTGTCGTTTTTTTCGA 3′.

In any of these sequences, all of the linkages may be all phosphorothioate bonds. In another embodiment, in any of these sequences, one or more of the linkages may be phosphodiester, preferably between the “C” and the “G” of the CpG motif making a semi-soft CpG oligonucleotide. In any of these sequences, an ethyl-uridine or a halogen may substitute for the 5′ T; examples of halogen substitutions include but are not limited to bromo-uridine or iodo-uridine substitutions.

Some non-limiting examples of B-Class oligonucleotides include:

(SEQ ID NO: 6)
5′ T*C*G*T*C*G*T*T*T*T*T*C*G*G*T*G*C*T*T*T*T 3′,
or
(SEQ ID NO: 7)
5′ T*C*G*T*C*G*T*T*T*T*T*C*G*G*T*C*G*T*T*T*T 3′,
or
(SEQ ID NO: 8)
5′ T*C*G*T*C*G*T*T*T*T*G*T*C*G*T*T*T*T*G*T*C*G*T*
T 3′,
or
(SEQ ID NO: 9)
5′ T*C*G*T*C*G*T*T*T*C*G*T*C*G*T*T*T*T*G*T*C*G*T*
T 3′,
or
(SEQ ID NO: 10)
5′ T*C*G*T*C*G*T*T*T*T*G*T*C*G*T*T*T*T*T*T*T*C*G*
A 3′.

wherein “*” refers to a phosphorothioate bond.

In an embodiment of the present invention, the immunogenic compositions as disclosed herein comprise a C class CpG oligonucleotide.

In an embodiment of the present invention, the immunogenic compositions as disclosed herein comprise a P class CpG oligonucleotide.

In one embodiment the oligonucleotide includes at least one phosphorothioate linkage.

In another embodiment all internucleotide linkages of the oligonucleotide are phosphorothioate linkages. In another embodiment the oligonucleotide includes at least one phosphodiester-like linkage. In another embodiment the phosphodiester-like linkage is a phosphodiester linkage. In another embodiment a lipophilic group is conjugated to the oligonucleotide. In one embodiment the lipophilic group is cholesterol.

In an embodiment, all the internucleotide linkage of the CpG oligonucleotides disclosed herein are phosphodiester bonds (“soft” oligonucleotides, as described in WO 2007/026190). In another embodiment, CpG oligonucleotides of the invention are rendered resistant to degradation (e.g., are stabilized). A “stabilized oligonucleotide” refers to an oligonucleotide that is relatively resistant to in vivo degradation (e.g., via an exo- or endo-nuclease). Nucleic acid stabilization can be accomplished via backbone modifications. Oligonucleotides having phosphorothioate linkages provide maximal activity and protect the oligonucleotide from degradation by intracellular exo- and endo-nucleases.

The immunostimulatory oligonucleotides may have a chimeric backbone, which have combinations of phosphodiester and phosphorothioate linkages. For purposes of the instant invention, a chimeric backbone refers to a partially stabilized backbone, wherein at least one internucleotide linkage is phosphodiester or phosphodiester-like, and wherein at least one other internucleotide linkage is a stabilized internucleotide linkage, wherein the at least one phosphodiester or phosphodiester-like linkage and the at least one stabilized linkage are different. When the phosphodiester linkage is preferentially located within the CpG motif such molecules are called “semi-soft” as described in WO 2007/026190.

Other modified oligonucleotides include combinations of phosphodiester, phosphorothioate, methylphosphonate, methylphosphorothioate, phosphorodithioate, and/or p-ethoxy linkages.

Mixed backbone modified ODN may be synthesized as described in WO 2007/026190. The size of the CpG oligonucleotide (i.e., the number of nucleotide residues along the length of the oligonucleotide) also may contribute to the stimulatory activity of the oligonucleotide. For facilitating uptake into cells, CpG oligonucleotide of the invention preferably have a minimum length of 6 nucleotide residues. Oligonucleotides of any size greater than 6 nucleotides (even many kb long) are capable of inducing an immune response if sufficient immunostimulatory motifs are present, because larger oligonucleotides are degraded inside cells. In certain embodiments, the CpG oligonucleotides are 6 to 100 nucleotides long, preferentially 8 to 30 nucleotides long. In important embodiments, nucleic acids and oligonucleotides of the invention are not plasmids or expression vectors.

In an embodiment, the CpG oligonucleotide disclosed herein comprise substitutions or modifications, such as in the bases and/or sugars as described at paragraphs 134 to 147 of WO 2007/026190.

In an embodiment, the CpG oligonucleotide of the present invention is chemically modified. Examples of chemical modifications are known to the skilled person and are described, for example in Uhlmann et al. (1990) Chem. Rev. 90:543; S. Agrawal, Ed., Humana Press, Totowa, USA 1993; Crooke. et al. (1996) Annu. Rev. Pharmacol. Toxicol. 36:107-129; and Hunziker et al., (1995) Mod. Synth. Methods 7:331-417. An oligonucleotide according to the invention may have one or more modifications, wherein each modification is located at a particular phosphodiester internucleoside bridge and/or at a particular β-D-ribose unit and/or at a particular natural nucleoside base position in comparison to an oligonucleotide of the same sequence which is composed of natural DNA or RNA.

In some embodiments of the invention, CpG-containing nucleic acids might be simply mixed with immunogenic carriers according to methods known to those skilled in the art (see, e.g., WO 03/024480).

In a particular embodiment of the present invention, any of the immunogenic composition disclosed herein comprises from 2 μg to 100 mg of CpG oligonucleotide, preferably from 0.1 mg to 50 mg CpG oligonucleotide, preferably from 0.2 mg to 10 mg CpG oligonucleotide, preferably from 0.3 mg to 5 mg CpG oligonucleotide, preferably from 0.3 mg to 5 mg CpG oligonucleotide, even preferably from 0.5 mg to 2 mg CpG oligonucleotide, even preferably from 0.75 mg to 1.5 mg CpG oligonucleotide. In a preferred embodiment, any of the immunogenic composition disclosed herein comprises about 1 mg CpG oligonucleotide.

5 Formulation

The immunogenic compositions of the invention may be formulated in liquid form (i.e., solutions or suspensions) or in a lyophilized form. Liquid formulations may advantageously be administered directly from their packaged form and are thus ideal for injection without the need for reconstitution in aqueous medium as otherwise required for lyophilized compositions of the invention.

Formulation of the immunogenic composition of the present invention can be accomplished using art-recognized methods. For instance, the individual pneumococcal conjugates can be formulated with a physiologically acceptable vehicle to prepare the composition. Examples of such vehicles include, but are not limited to, water, buffered saline, polyols (e.g., glycerol, propylene glycol, liquid polyethylene glycol) and dextrose solutions.

The present disclosure provides an immunogenic composition comprising any of combination of glycoconjugates disclosed herein and a pharmaceutically acceptable excipient, carrier, or diluent.

In an embodiment, the immunogenic composition of the invention is in liquid form, preferably in aqueous liquid form.

Immunogenic compositions of the disclosure may comprise one or more of a buffer, a salt, a divalent cation, a non-ionic detergent, a cryoprotectant such as a sugar, and an anti-oxidant such as a free radical scavenger or chelating agent, or any multiple combinations thereof.

In an embodiment, the immunogenic composition of the invention comprises a buffer. In an embodiment, said buffer has a pKa of about 3.5 to about 7.5. In some embodiments, the buffer is phosphate, succinate, histidine or citrate. In certain embodiments, the buffer is succinate at a final concentration of 1 mM to 10 mM. In one particular embodiment, the final concentration of the succinate buffer is about 5 mM.

In an embodiment, the immunogenic composition of the invention comprises a salt. In some embodiments, the salt is selected from the groups consisting of magnesium chloride, potassium chloride, sodium chloride and a combination thereof. In one particular embodiment, the salt is sodium chloride. In one particular embodiment, the immunogenic composition of the invention comprises sodium chloride at 150 mM.

In an embodiment, the immunogenic compositions of the invention comprise a surfactant. In an embodiment, the surfactant is selected from the group consisting of polysorbate 20 (TWEEN™ 20), polysorbate 40 (TWEEN™40), polysorbate 60 (TWEEN™ 60), polysorbate 65 (TWEEN™65), polysorbate 80 (TWEEN™80), polysorbate 85 (TWEEN™85), TRITON™ N-1 01, TRITON™ X-100, oxtoxynol 40, nonoxynol-9, triethanolamine, triethanolamine polypeptide oleate, polyoxyethylene-660 hydroxystearate (PEG-15, Solutol H 15), polyoxyethylene-35-ricinoleate (CREMOPHOR® EL), soy lecithin and a poloxamer. In one particular embodiment, the surfactant is polysorbate 80. In some said embodiment, the final concentration of polysorbate 80 in the formulation is at least 0.0001% to 10% polysorbate 80 weight to weight (w/w). In some said embodiments, the final concentration of polysorbate 80 in the formulation is at least 0.001% to 1% polysorbate 80 weight to weight (w/w). In some said embodiments, the final concentration of polysorbate 80 in the formulation is at least 0.01% to 1% polysorbate 80 weight to weight (w/w). In other embodiments, the final concentration of polysorbate 80 in the formulation is 0.01%, 0.02%, 0.03%, 0.04%, 0.05%, 0.06%, 0.07%, 0.08%, 0.09% or 0.1% polysorbate 80 (w/w). In another embodiment, the final concentration of the polysorbate 80 in the formulation is 0.02% polysorbate 80 (w/w). In another embodiment, the final concentration of the polysorbate 80 in the formulation is 0.01% polysorbate 80 (w/w). In another embodiment, the final concentration of the polysorbate 80 in the formulation is 0.03% polysorbate 80 (w/w). In another embodiment, the final concentration of the polysorbate 80 in the formulation is 0.04% polysorbate 80 (w/w). In another embodiment, the final concentration of the polysorbate 80 in the formulation is 0.05% polysorbate 80 (w/w). In another embodiment, the final concentration of the polysorbate 80 in the formulation is 1% polysorbate 80 (w/w). In one particular embodiment, the surfactant is polysorbate 20. In some said embodiment, the final concentration of polysorbate 20 in the formulation is at least 0.0001% to 10% polysorbate 20 weight to weight (w/w). In some said embodiments, the final concentration of polysorbate 20 in the formulation is at least 0.001% to 1% polysorbate 20 weight to weight (w/w). In some said embodiments, the final concentration of polysorbate 20 in the formulation is at least 0.01% to 1% polysorbate 20 weight to weight (w/w). In other embodiments, the final concentration of polysorbate 20 in the formulation is 0.01%, 0.02%, 0.03%, 0.04%, 0.05%, 0.06%, 0.07%, 0.08%, 0.09% or 0.1% polysorbate 20 (w/w). In another embodiment, the final concentration of the polysorbate 20 in the formulation is 0.02% polysorbate 20 (w/w). In another embodiment, the final concentration of the polysorbate 20 in the formulation is 0.01% polysorbate 20 (w/w). In another embodiment, the final concentration of the polysorbate 20 in the formulation is 0.03% polysorbate 20 (w/w). In another embodiment, the final concentration of the polysorbate 20 in the formulation is 0.04% polysorbate 80 (w/w). In another embodiment, the final concentration of the polysorbate 20 in the formulation is 0.05% polysorbate 20 (w/w). In another embodiment, the final concentration of the polysorbate 20 in the formulation is 1% polysorbate 20 (w/w).

In one particular embodiment, the surfactant is polysorbate 40. In some said embodiment, the final concentration of polysorbate 40 in the formulation is at least 0.0001% to 10% polysorbate 40 weight to weight (w/w). In some said embodiments, the final concentration of polysorbate 40 in the formulation is at least 0.001% to 1% polysorbate 40 weight to weight (w/w). In some said embodiments, the final concentration of polysorbate 40 in the formulation is at least 0.01% to 1% polysorbate 40 weight to weight (w/w). In other embodiments, the final concentration of polysorbate 40 in the formulation is 0.01%, 0.02%, 0.03%, 0.04%, 0.05%, 0.06%, 0.07%, 0.08%, 0.09% or 0.1% polysorbate 40 (w/w). In another embodiment, the final concentration of the polysorbate 40 in the formulation is 1% polysorbate 40 (w/w).

In one particular embodiment, the surfactant is polysorbate 60. In some said embodiment, the final concentration of polysorbate 60 in the formulation is at least 0.0001% to 10% polysorbate 60 weight to weight (w/w). In some said embodiments, the final concentration of polysorbate 60 in the formulation is at least 0.001% to 1% polysorbate 60 weight to weight (w/w). In some said embodiments, the final concentration of polysorbate 60 in the formulation is at least 0.01% to 1% polysorbate 60 weight to weight (w/w). In other embodiments, the final concentration of polysorbate 60 in the formulation is 0.01%, 0.02%, 0.03%, 0.04%, 0.05%, 0.06%, 0.07%, 0.08%, 0.09% or 0.1% polysorbate 60 (w/w). In another embodiment, the final concentration of the polysorbate 60 in the formulation is 1% polysorbate 60 (w/w).

In one particular embodiment, the surfactant is polysorbate 65. In some said embodiment, the final concentration of polysorbate 65 in the formulation is at least 0.0001% to 10% polysorbate 65 weight to weight (w/w). In some said embodiments, the final concentration of polysorbate 65 in the formulation is at least 0.001% to 1% polysorbate 65 weight to weight (w/w). In some said embodiments, the final concentration of polysorbate 65 in the formulation is at least 0.01% to 1% polysorbate 65 weight to weight (w/w). In other embodiments, the final concentration of polysorbate 65 in the formulation is 0.01%, 0.02%, 0.03%, 0.04%, 0.05%, 0.06%, 0.07%, 0.08%, 0.09% or 0.1% polysorbate 65 (w/w). In another embodiment, the final concentration of the polysorbate 65 in the formulation is 1% polysorbate 65 (w/w).

In one particular embodiment, the surfactant is polysorbate 85. In some said embodiment, the final concentration of polysorbate 85 in the formulation is at least 0.0001% to 10% polysorbate 85 weight to weight (w/w). In some said embodiments, the final concentration of polysorbate 85 in the formulation is at least 0.001% to 1% polysorbate 85 weight to weight (w/w). In some said embodiments, the final concentration of polysorbate 85 in the formulation is at least 0.01% to 1% polysorbate 85 weight to weight (w/w). In other embodiments, the final concentration of polysorbate 85 in the formulation is 0.01%, 0.02%, 0.03%, 0.04%, 0.05%, 0.06%, 0.07%, 0.08%, 0.09% or 0.1% polysorbate 85 (w/w). In another embodiment, the final concentration of the polysorbate 85 in the formulation is 1% polysorbate 85 (w/w).

In certain embodiments, the immunogenic composition of the invention has a pH of 5.5 to 7.5, more preferably a pH of 5.6 to 7.0, even more preferably a pH of 5.8 to 6.0.

In one embodiment, the present invention provides a container filled with any of the immunogenic compositions disclosed herein. In one embodiment, the container is selected from the group consisting of a vial, a syringe, a flask, a fermentor, a bioreactor, a bag, a jar, an ampoule, a cartridge and a disposable pen. In certain embodiments, the container is siliconized.

In an embodiment, the container of the present invention is made of glass, metals (e.g., steel, stainless steel, aluminum, etc.) and/or polymers (e.g., thermoplastics, elastomers, thermoplastic-elastomers). In an embodiment, the container of the present invention is made of glass.

In one embodiment, the present invention provides a syringe filled with any of the immunogenic compositions disclosed herein. In certain embodiments, the syringe is siliconized and/or is made of glass.

A typical dose of the immunogenic composition of the invention for injection has a volume of 0.1 mL to 2 mL, more preferably 0.2 mL to 1 mL, even more preferably a volume of about 0.5 mL.

Therefore, the container or syringe as defined above is filed with a volume of 0.1 mL to 2 mL, more preferably 0.2 mL to 1 mL, even more preferably a volume of about 0.5 mL of any of the immunogenic composition defined herein.

6 Ability of the Immunogenic Compositions or Set of Immunogenic Compositions of the Invention to Elicit Cross-Reactive Antibodies

In an embodiment, the immunogenic composition or set of immunogenic compositions of the invention is able to elicit IgG antibodies in human which are capable of binding S. pneumoniae serotypes 15A, 15B and 15C polysaccharide as determined by ELISA assay.

In the ELISA (Enzyme-linked Immunosorbent Assay) method, antibodies from the sera of vaccinated subjects are incubated with polysaccharides which have been adsorbed to a solid support. The bound antibodies are detected using enzyme-conjugated secondary detection antibodies.

In an embodiment said ELISA assay is the standardized ELISA assay as defined by the WHO in the “Training Manual For Enzyme Linked Immunosorbent Assay For The Quantitation Of Streptococcus Pneumoniae Serotype Specific IgG (Pn PS ELISA).” (available at https://www.vaccine.uab.edu/uploads/mdocs/ELISAProtocol(007sp).pdf, accessed on Sep. 16, 2020).

The ELISA measures type specific IgG anti-S. pneumoniae capsular polysaccharide (PS) antibodies present in human serum. When dilutions of human sera are added to type-specific capsular PS-coated microtiter plates, antibodies specificforthat capsular PS bind to the microtiter plates. The antibodies bound to the plates are detected using a goat anti-human IgG alkaline phosphatase-labeled antibody followed by a p-nitrophenyl phosphate substrate. The optical density of the colored end product is proportional to the amount of anticapsular PS antibody present in the serum.

In an embodiment, the immunogenic composition or set of immunogenic compositions of the invention is able to elicit IgG antibodies in human which are capable of binding S. pneumoniae serotype 15A polysaccharide at a concentration of at least 0.05 μg/ml, 0.1 μg/ml, 0.2 μg/ml, 0.3 μg/ml, 0.35 μg/ml, 0.4 μg/ml or 0.5 μg/ml as determined by ELISA assay. In an embodiment, the immunogenic composition or set of immunogenic compositions of the invention is able to elicit IgG antibodies in human which are capable of binding S. pneumoniae serotype 15A polysaccharide at a concentration of at least 0.3 μg/ml as determined by ELISA assay. In an embodiment, the immunogenic composition or set of immunogenic compositions of the invention is able to elicit IgG antibodies in human which are capable of binding S. pneumoniae serotype 15A polysaccharide at a concentration of at least 0.35 μg/ml as determined by ELISA assay.

In an embodiment, the immunogenic composition or set of immunogenic compositions of the invention is able to elicit IgG antibodies in human which are capable of binding S. pneumoniae serotype 15B polysaccharide at a concentration of at least 0.05 μg/ml, 0.1 μg/ml, 0.2 μg/ml, 0.3 μg/ml, 0.35 μg/ml, 0.4 μg/ml or 0.5 μg/ml as determined by ELISA assay. In an embodiment, the immunogenic composition or set of immunogenic compositions of the invention is able to elicit IgG antibodies in human which are capable of binding S. pneumoniae serotype 15B polysaccharide at a concentration of at least 0.3 μg/ml as determined by ELISA assay. In an embodiment, the immunogenic composition or set of immunogenic compositions of the invention is able to elicit IgG antibodies in human which are capable of binding S. pneumoniae serotype 15B polysaccharide at a concentration of at least 0.35 μg/ml as determined by ELISA assay.

In an embodiment, the immunogenic composition or set of immunogenic compositions of the invention is able to elicit IgG antibodies in human which are capable of binding S. pneumoniae serotype 15C polysaccharide at a concentration of at least 0.05 μg/ml, 0.1 μg/ml, 0.2 μg/ml, 0.3 μg/ml, 0.35 μg/ml, 0.4 μg/ml or 0.5 μg/ml as determined by ELISA assay. In an embodiment, the immunogenic composition or set of immunogenic compositions of the invention is able to elicit IgG antibodies in human which are capable of binding S. pneumoniae serotype 15C polysaccharide at a concentration of at least 0.3 μg/ml as determined by ELISA assay. In an embodiment, the immunogenic composition or set of immunogenic compositions of the invention is able to elicit IgG antibodies in human which are capable of binding S. pneumoniae serotype 15C polysaccharide at a concentration of at least 0.35 μg/ml as determined by ELISA assay.

In an embodiment, the immunogenic composition or set of immunogenic compositions of the invention is able to elicit functional antibodies in humans which are capable of killing S. pneumoniae serotype 15A, 15B and 15C as determined by in vitro opsonophagocytic assay (OPA) (see Example 1). In an embodiment, the immunogenic composition or set of immunogenic compositions of the invention is able to elicit functional antibodies in humans which are capable of killing S. pneumoniae serotype 15A as determined by in vitro opsonophagocytic assay (OPA). In an embodiment, the immunogenic composition or set of immunogenic compositions of the invention is able to elicit functional antibodies in humans which are capable of killing S. pneumoniae serotype 15B as determined by in vitro opsonophagocytic assay (OPA). In an embodiment, the immunogenic composition or set of immunogenic compositions of the invention is able to elicit functional antibodies in human which are capable of killing S. pneumoniae serotype 15C as determined by in vitro opsonophagocytic assay (OPA).

The pneumococcal opsonophagocytic assay (OPA), which measures killing of S. pneumoniae cells by phagocytic effector cells in the presence of functional antibody and complement, is considered to be an important surrogate for evaluating the effectiveness of pneumococcal vaccines.

In vitro opsonophagocytic assay (OPA) can be conducted by incubating together a mixture of Streptococcus pneumoniae cells, a heat inactivated human serum to be tested, differentiated HL-60 cells (phagocytes) and an exogenous complement source (e.g., baby rabbit complement). Opsonophagocytosis proceeds during incubation and bacterial cells that are coated with antibody and complement are killed upon opsonophagocytosis. Colony forming units (cfu) of surviving bacteria that escape from opsonophagocytosis are determined by plating the assay mixture. The OPA titer is defined as the reciprocal dilution that results in a 50% reduction in bacterial count over control wells without test serum. The OPA titer is interpolated from the two dilutions that encompass this 50% killing cut-off.

An endpoint titer of 1:8 or greater is considered a positive result in these killing type OPA. In an embodiment, the immunogenic composition or set of immunogenic compositions of the invention is able to elicit a titer of at least 1:8 against S. pneumoniae serotype 15A, 15B and 15C in at least 50% of the subjects as determined by in vitro opsonophagocytic killing assay (OPA). In an embodiment, the immunogenic composition or set of immunogenic compositions of the invention is able to elicit a titer of at least 1:8 against S. pneumoniae serotype 15A, 15B and 15C in at least 60% of the subjects as determined by in vitro opsonophagocytic killing assay (OPA).

In an embodiment, the immunogenic composition or set of immunogenic compositions of the invention is able to elicit a titer of at least 1:8 against S. pneumoniae serotype 15A, 15B and 15C in at least 80% of the subjects as determined by in vitro opsonophagocytic killing assay (OPA). In an embodiment, the immunogenic composition or set of immunogenic compositions of the invention is able to elicit a titer of at least 1:8 against S. pneumoniae serotype 15A, 15B and 15C in at least 90% of the subjects as determined by in vitro opsonophagocytic killing assay (OPA). In an embodiment, the immunogenic composition or set of immunogenic compositions of the invention is able to elicit a titer of at least 1:8 against S. pneumoniae serotype 15A, 15B and 15C in at least 95%, of the subjects as determined by in vitro opsonophagocytic killing assay (OPA).

In an embodiment, the immunogenic composition or set of immunogenic compositions of the invention is able to elicit a titer of at least 1:8 against S. pneumoniae serotype 15A, 15B and 15C in at least 98% of the subjects as determined by in vitro opsonophagocytic killing assay (OPA).

In some embodiment, the subjects may have serotype specific OPA titers prior to pneumococcal vaccination due for example to natural exposures to S. pneumoniae (e.g., in case of adult subjects).

Therefore, comparison of OPA activity of pre- and post-immunization serum with the immunogenic composition of the invention can be conducted and compared for their response to serotypes 15A, 15B, and 15C to assess the potential increase of responders (see Example 1).

In an embodiment the immunogenic composition or set of immunogenic compositions of the invention significantly increases the proportion of responders (i.e., individual with a serum having a titer of at least 1:8 as determined by in vitro OPA) as compared to the pre-immunized population.

Therefore in an embodiment, the immunogenic composition or set of immunogenic compositions of the invention is able to significantly increase the proportion of responders against S. pneumoniae serotypes 15A, 15B and 15C (i.e., individual with a serum having a titer of at least 1:8 as determined by in vitro OPA) as compared to the pre-immunized population.

Comparison of OPA activity of pre- and post-immunization serum with the immunogenic composition of the invention can also be done by comparing the potential increase in OPA titers.

Therefore, comparison of OPA activity of pre- and post-immunization serum with the immunogenic composition or set of immunogenic compositions of the invention can be conducted and compared for their response to serotypes 15A, 15B, and 15C to assess the potential for increase in OPA titers (see Example 1).

In an embodiment the immunogenic composition or set of immunogenic compositions of the invention are able to significantly increase the OPA titer of human subjects as compared to the pre-immunized population.

Therefore, in an embodiment, the immunogenic composition or set of immunogenic compositions of the invention is able to significantly increase the OPA titers of human subjects against S. pneumoniae serotype 15A, 15B and 15C as compared to the pre-immunized population. In an embodiment, the fold-rise in OPA titer against S. pneumoniae serotype 15A is at least 2.0, 3.0, 4.0, 5.0, 6.0, 7.0, 7.5 or 8.0. In an embodiment, the fold-rise in OPA titer against S. pneumoniae serotype 15A is at least 2.0. In an embodiment, the fold-rise in OPA titer against S. pneumoniae serotype 15A is at least 3.0. In an embodiment, the fold-rise in OPA titer against S. pneumoniae serotype 15A is at least 4.0. In an embodiment, the fold-rise in OPA titer against S. pneumoniae serotype 15B is at least 2.0, 3.0, 4.0, 5.0, 6.0, 7.0, 7.5 or 8.0. In an embodiment, the fold-rise in OPA titer against S. pneumoniae serotype 15B is at least 2.0. In an embodiment, the fold-rise in OPA titer against S. pneumoniae serotype 15B is at least 3.0. In an embodiment, the fold-rise in OPA titer against S. pneumoniae serotype 15B is at least 4.0. In an embodiment, the fold-rise in OPA titer against S. pneumoniae serotype 15C is at least 2.0, 3.0, 4.0, 5.0, 6.0, 7.0, 7.5 or 8.0. In an embodiment, the fold-rise in OPA titer against S. pneumoniae serotype 15C is at least 2.0. In an embodiment, the fold-rise in OPA titer against S. pneumoniae serotype 15C is at least 3.0. In an embodiment, the fold-rise in OPA titer against S. pneumoniae serotype 15C is at least 4.0.

In an embodiment, the immunogenic composition or set of immunogenic compositions of the invention is able to significantly increase the OPA titers of human subjects against S. pneumoniae serotypes 15A, 15B and 15C as compared to the pre-immunized population.

In an embodiment, the fold-rise in OPA titer against S. pneumoniae serotype 15A is at least 2.0, 3.0, 4.0, 5.0, 6.0, 7.0, 7.5 or 8.0, the fold-rise in OPA titer against S. pneumoniae serotype 15B is at least at least 2.0, 3.0, 4.0, 5.0, 6.0, 7.0, 7.5 or 8.0 and the fold-rise in OPA titer against S. pneumoniae serotype 15C is at least at least 2.0, 3.0, 4.0, 5.0, 6.0, 7.0, 7.5 or 8.0. In an embodiment, the fold-rise in OPA titer against S. pneumoniae serotype 15A is at least 2.0, the fold-rise in OPA titer against S. pneumoniae serotype 15B is at least 2.0 and the fold-rise in OPA titer against S. pneumoniae serotype 15C is at least 2.0. In an embodiment, the fold-rise in OPA titer against S. pneumoniae serotype 15A is at least 3.0, the fold-rise in OPA titer against S. pneumoniae serotype 15B is at least 3.0 and the fold-rise in OPA titer against S. pneumoniae serotype 15C is at least 3.0. In an embodiment, the fold-rise in OPA titer against S. pneumoniae serotype 15A is at least 4.0, the fold-rise in OPA titer against S. pneumoniae serotype 15B is at least 4.0 and the fold-rise in OPA titer against S. pneumoniae serotype 15C is at least 4.0.

7 Uses of the Immunogenic Compositions and Set of Immunogenic Compositions of the Invention

In an embodiment, the immunogenic compositions or set of immunogenic compositions disclosed herein are for use as a medicament.

In particular, the immunogenic compositions or set of immunogenic compositions described herein may be used to prevent, treat or ameliorate a S. pneumoniae infection, disease or condition in a subject.

Thus, in one aspect, the invention provides a method of preventing, treating or ameliorating an infection, disease or condition associated with S. pneumoniae in a subject, comprising administering to the subject an immunologically effective amount of an immunogenic composition of the invention.

In one aspect, the invention provides a method of preventing, treating or ameliorating an infection, disease or condition associated with S. pneumoniae in a subject, comprising administering to the subject an immunologically effective amount of a set of immunogenic compositions of the invention.

In one aspect, the invention provides a method of preventing, treating or ameliorating an infection, disease or condition associated with S. pneumoniae serotypes 15A, 15B and 15C in a subject, comprising administering to the subject an immunologically effective amount of an immunogenic composition of the invention.

In one aspect, the invention provides a method of preventing, treating or ameliorating an infection, disease or condition associated with S. pneumoniae serotypes 15A, 15B and 15C in a subject, comprising administering to the subject an immunologically effective amount of a set of immunogenic compositions of the invention.

In one aspect, the invention provides a method of inducing an immune response to S. pneumoniae serotypes 15A, 15B and 15C in a subject, comprising administering to the subject an immunologically effective amount of an immunogenic composition of the invention.

In one aspect, the invention provides a method of inducing an immune response to S. pneumoniae serotypes 15A, 15B and 15C in a subject, comprising administering to the subject an immunologically effective amount of a set of immunogenic compositions of the invention.

In one aspect, the immunogenic compositions of the present invention are for use in a method for preventing, treating or ameliorating an infection, disease or condition caused by S. pneumoniae serotypes 15A, 15B and 15C in a subject.

In one aspect, the sets of immunogenic compositions of the present invention are for use in a method for preventing, treating or ameliorating an infection, disease or condition caused by S. pneumoniae serotypes 15A, 15B and 15C in a subject.

In an embodiment, any of the immunogenic composition disclosed herein is for use in a method of immunizing a subject against infection by S. pneumoniae serotypes 15A, 15B and 15C.

In an embodiment, any of the set of immunogenic compositions disclosed herein is for use in a method of immunizing a subject against infection by S. pneumoniae serotypes 15A, 15B and 15C.

In one aspect, the present invention is directed toward the use of the immunogenic composition disclosed herein for the manufacture of a medicament for preventing, treating or ameliorating an infection, disease or condition caused by S. pneumoniae serotypes 15A, 15B and 15C in a subject.

In one aspect, the present invention is directed toward the use of the set of immunogenic compositions disclosed herein for the manufacture of a medicament for preventing, treating or ameliorating an infection, disease or condition caused by S. pneumoniae serotypes 15A, 15B and 15C in a subject.

In an embodiment, the present invention is directed toward the use of the immunogenic composition disclosed herein for the manufacture of a medicament for immunizing a subject against infection by S. pneumoniae serotype 15A, 15B and 15C.

In an embodiment, the present invention is directed toward the use of the set of immunogenic compositions disclosed herein for the manufacture of a medicament for immunizing a subject against infection by S. pneumoniae serotype 15A, 15B and 15C.

In one aspect, the present invention provides a method for inducing an immune response to S. pneumoniae serotypes 15A, 15B and 15C in a subject. In an embodiment, said method comprises administering to a subject an immunogenic composition as disclosed herein. In another embodiment, said method comprises administering to a subject a set of immunogenic compositions as disclosed herein.

In an embodiment, the immunogenic compositions disclosed herein are vaccines. In an embodiment, the set of immunogenic compositions disclosed herein are a set of vaccines (i.e. the immunogenic compositions of the set of immunogenic compositions are vaccines).

In an embodiment, the immunogenic compositions disclosed herein are for use as a vaccine. In an embodiment, the set of immunogenic compositions disclosed herein are for use as a vaccine. More particularly, the immunogenic compositions or sets of immunogenic compositions described herein may be used to prevent serotypes 15A, 15B and 15C S. pneumoniae infections in a subject. Thus in one aspect, the invention provides a method of preventing, an infection by serotypes 15A, 15B and 15C S. pneumoniae in a subject, comprising administering to the subject an immunologically effective amount of an immunogenic composition or a set of immunogenic compositions of the invention. In some such embodiments, the infection is selected from the group consisting of pneumonia, sinusitis, otitis media, acute otitis media, meningitis, bacteremia, sepsis, pleural empyema, conjunctivitis, osteomyelitis, septic arthritis, endocarditis, peritonitis, pericarditis, mastoiditis, cellulitis, soft tissue infection and brain abscess. In some such embodiments, the infection is pneumonia, sinusitis, otitis media, acute otitis media, meningitis, bacteremia, sepsis, pleural empyema, conjunctivitis, osteomyelitis, septic arthritis, endocarditis, peritonitis, pericarditis, mastoiditis, cellulitis, soft tissue infection or brain abscess. In one aspect, the subject to be vaccinated is a mammal, such as a human, cat, sheep, pig, horse, bovine or dog. Preferably, said subject is a human subject.

In one aspect, the immunogenic compositions or sets of immunogenic compositions disclosed herein are for use in a method of preventing, treating or ameliorating an infection, disease or condition associated S. pneumoniae with serotypes 15A, 15B and 15C in a subject. In some such embodiments, the infection, disease or condition is selected from the group consisting of pneumonia, sinusitis, otitis media, acute otitis media, meningitis, bacteremia, sepsis, pleural empyema, conjunctivitis, osteomyelitis, septic arthritis, endocarditis, peritonitis, pericarditis, mastoiditis, cellulitis, soft tissue infection and brain abscess. In some such embodiments, the infection, disease or condition is pneumonia, sinusitis, otitis media, acute otitis media, meningitis, bacteremia, sepsis, pleural empyema, conjunctivitis, osteomyelitis, septic arthritis, endocarditis, peritonitis, pericarditis, mastoiditis, cellulitis, soft tissue infection or brain abscess. In one aspect, the subject is a mammal, such as a human, cat, sheep, pig, horse, bovine or dog. Preferably, said subject is a human subject.

In an aspect, the immunogenic compositions or sets of immunogenic compositions disclosed herein are for use in a method of preventing, an infection by serotypes 15A, 15B and 15C of S. pneumoniae in a subject. In some such embodiments, the infection is selected from the group consisting of pneumonia, sinusitis, otitis media, acute otitis media, meningitis, bacteremia, sepsis, pleural empyema, conjunctivitis, osteomyelitis, septic arthritis, endocarditis, peritonitis, pericarditis, mastoiditis, cellulitis, soft tissue infection and brain abscess. In one aspect, the subject to be vaccinated is a mammal, such as a human, cat, sheep, pig, horse, bovine or dog. Preferably, said subject is a human subject.

In one aspect, the present invention is directed toward the use of the immunogenic composition or set of immunogenic compositions disclosed herein for the manufacture of a medicament for preventing, treating or ameliorating an infection, disease or condition associated S. pneumoniae with serotypes 15A, 15B and 15C in a subject. Preferably, said subject is a human subject. In some such embodiments, the infection, disease or condition is selected from the group consisting of pneumonia, sinusitis, otitis media, acute otitis media, meningitis, bacteremia, sepsis, pleural empyema, conjunctivitis, osteomyelitis, septic arthritis, endocarditis, peritonitis, pericarditis, mastoiditis, cellulitis, soft tissue infection and brain abscess. In some such embodiments, the infection, disease or condition is pneumonia, sinusitis, otitis media, acute otitis media, meningitis, bacteremia, sepsis, pleural empyema, conjunctivitis, osteomyelitis, septic arthritis, endocarditis, peritonitis, pericarditis, mastoiditis, cellulitis, soft tissue infection or brain abscess.

In an aspect, the present invention is directed toward the use of the immunogenic composition or set of immunogenic compositions disclosed herein for the manufacture of a medicament for preventing, an infection by serotypes 15A, 15B and 15C of S. pneumoniae in a subject. In some such embodiments, the infection is selected from the group consisting of pneumonia, sinusitis, otitis media, acute otitis media, meningitis, bacteremia, sepsis, pleural empyema, conjunctivitis, osteomyelitis, septic arthritis, endocarditis, peritonitis, pericarditis, mastoiditis, cellulitis, soft tissue infection and brain abscess. In one aspect, the subject to be vaccinated is a mammal, such as a human, cat, sheep, pig, horse, bovine or dog. Preferably, said subject is a human subject.

The immunogenic compositions or set of immunogenic compositions of the present invention can be used to protect or treat a human susceptible to S. pneumoniae serotypes 15A, 15B and 15C infection, by means of administering the immunogenic compositions or sets of immunogenic compositions via a systemic or mucosal route. In an embodiment, the immunogenic compositions or sets of immunogenic compositions disclosed herein are administered by intramuscular, intraperitoneal, intradermal or subcutaneous routes. In an embodiment, the immunogenic compositions or sets of immunogenic compositions disclosed herein are administered by intramuscular, intraperitoneal, intradermal or subcutaneous injection. In an embodiment, the immunogenic compositions or sets of immunogenic compositions disclosed herein are administered by intramuscular or subcutaneous injection. In an embodiment, the immunogenic compositions or sets of immunogenic compositions disclosed herein are administered by intramuscular injection. In an embodiment, the immunogenic compositions or sets of immunogenic compositions disclosed herein are administered by subcutaneous injection.

8 Subject to be Treated with the Immunogenic Compositions or Sets of Immunogenic Compositions of the Invention

As disclosed herein, the immunogenic compositions or sets of immunogenic compositions described herein may be used in various therapeutic or prophylactic methods for preventing, treating or ameliorating a bacterial infection, disease or condition in a subject.

In a preferred embodiment, said subject is a human. In a most preferred embodiment, said subject is a newborn (i.e., under three months of age), an infant (i.e., from 3 months to one year of age) or a toddler (i.e., from one year to four years of age).

In an embodiment, the immunogenic compositions disclosed herein are for use as a vaccine.

In such embodiment, the subject to be vaccinated may be less than 1 year of age. For example, the subject to be vaccinated can be about 1, about 2, about 3, about 4, about 5, about 6, about 7, about 8, about 9, about 10, about 11 or about 12 months of age. In an embodiment, the subject to be vaccinated is about 2, 4 or 6 months of age. In another embodiment, the subject to be vaccinated is less than 2 years of age. For example the subject to be vaccinated can be about 12 to about 15 months of age. In some cases, as little as one dose of the immunogenic composition or set of immunogenic compositions according to the invention is needed, but under some circumstances, a second, third or fourth dose may be given (see section 9 below).

In an embodiment of the present invention, the subject to be vaccinated is a human adult 50 years of age or older, more preferably a human adult 55 years of age or older. In an embodiment, the subject to be vaccinated is a human adult 65 years of age or older, 70 years of age or older, 75 years of age or older or 80 years of age or older.

In an embodiment the subject to be vaccinated is an immunocompromised individual, in particular a human. An immunocompromised individual is generally defined as a person who exhibits an attenuated or reduced ability to mount a normal humoral or cellular defense to challenge by infectious agents.

In an embodiment of the present invention, the immunocompromised subject to be vaccinated suffers from a disease or condition that impairs the immune system and results in an antibody response that is insufficient to protect against or treat pneumococcal disease.

In an embodiment, said disease is a primary immunodeficiency disorder. Preferably, said primary immunodeficiency disorder is selected from the group consisting of: combined T- and B-cell immunodeficiencies, antibody deficiencies, well-defined syndromes, immune dysregulation diseases, phagocyte disorders, innate immunity deficiencies, autoinflammatory disorders, and complement deficiencies. In an embodiment, said primary immunodeficiency disorder is combined T- and B-cell immunodeficiencies, antibody deficiencies, well-defined syndromes, immune dysregulation diseases, phagocyte disorders, innate immunity deficiencies, autoinflammatory disorders, or complement deficiencies. In an embodiment, said primary immunodeficiency disorder is selected from the one disclosed on page 24, line 11, to page 25, line 19, of WO 2010/125480.

In a particular embodiment of the present invention, the immunocompromised subject to be vaccinated suffers from a disease selected from the groups consisting of: HIV-infection, acquired immunodeficiency syndrome (AIDS), cancer, chronic heart or lung disorders, congestive heart failure, diabetes mellitus, chronic liver disease, alcoholism, cirrhosis, spinal fluid leaks, cardiomyopathy, chronic bronchitis, emphysema, chronic obstructive pulmonary disease (COPD), spleen dysfunction (such as sickle cell disease), lack of spleen function (asplenia), blood malignancy, leukemia, multiple myeloma, Hodgkin's disease, lymphoma, kidney failure, nephrotic syndrome and asthma.

In an embodiment of the present invention, the immunocompromised subject to be vaccinated suffers from malnutrition.

In a particular embodiment of the present invention, the immunocompromised subject to be vaccinated is taking a drug or treatment that lowers the body's resistance to infection.

In an embodiment, said drug is selected from the one disclosed on page 26, line 33, to page 26, line 4, of WO 2010/125480.

In a particular embodiment of the present invention, the immunocompromised subject to be vaccinated is a smoker.

In a particular embodiment of the present invention, the immunocompromised subject to be vaccinated has a white blood cell count (leukocyte count) below 5×10⁹ cells per liter, or below 4×10⁹ cells per liter, or below 3×10⁹ cells per liter, or below 2×10⁹ cells per liter, or below 1×10⁹ cells per liter, or below 0.5×10⁹ cells per liter, or below 0.3×10⁹ cells per liter, or below 0.1×10⁹ cells per liter.

White blood cell count (leukocyte count): The number of white blood cells (WBC) in the blood. The WBC is usually measured as part of the CBC (complete blood count). White blood cells are the infection-fighting cells in the blood and are distinct from the red (oxygen-carrying) blood cells known as erythrocytes. There are different types of white blood cells, including neutrophils (polymorphonuclear leukocytes; PMN), band cells (slightly immature neutrophils), T-type lymphocytes (T-cells), B-type lymphocytes (B-cells), monocytes, eosinophils, and basophils. All the types of white blood cells are reflected in the white blood cell count. The normal range for the white blood cell count is usually between 4,300 and 10,800 cells per cubic millimeter of blood. This can also be referred to as the leukocyte count and can be expressed in international units as 4.3-10.8×10⁹ cells per liter.

In a particular embodiment of the present invention, the immunocompromised subject to be vaccinated suffers from neutropenia. In a particular embodiment of the present invention, the immunocompromised subject to be vaccinated has a neutrophil count below 2×10⁹ cells per liter, or below 1×10⁹ cells per liter, or below 0.5×10⁹ cells per liter, or below 0.1×10⁹ cells per liter, or below 0.05×10⁹ cells per liter.

A low white blood cell count or “neutropenia” is a condition characterized by abnormally low levels of neutrophils in the circulating blood. Neutrophils are a specific kind of white blood cell that help prevent and fight infections. The most common reason that cancer patients experience neutropenia is as a side effect of chemotherapy. Chemotherapy-induced neutropenia increases a patient's risk of infection and disrupts cancer treatment.

In a particular embodiment of the present invention, the immunocompromised subject to be vaccinated has a CD4+ cell count below 500/mm3, or CD4+ cell count below 300/mm3, or CD4+ cell count below 200/mm3, CD4+ cell count below 100/mm3, CD4+ cell count below 75/mm3, or CD4+ cell count below 50/mm3.

CD4 cell tests are normally reported as the number of cells in mm3. Normal CD4 counts are between 500 and 1600, and CD8 counts are between 375 and 1100. CD4 counts drop dramatically in people with HIV.

In an embodiment of the invention, any of the immunocompromised subject disclosed herein is a human male or a human female. In an embodiment of the invention, any of the immunocompromised subject disclosed herein is a human male. In an embodiment of the invention, any of the immunocompromised subject disclosed herein is a human female.

9 Regimen of Administration of the Immunogenic Composition of the Invention

In some cases, as little as one dose of the immunogenic composition according to the invention is needed, but under some circumstances, such as conditions of greater immune deficiency, a second, third or fourth dose may be given. Following an initial vaccination, subjects can receive one or several booster immunizations adequately spaced.

In an embodiment, the schedule of vaccination of the immunogenic composition according to the invention is a single dose. In a particular embodiment, said single dose schedule is for healthy persons being at least 2 years of age.

In an embodiment, the schedule of vaccination of the immunogenic composition according to the invention is a multiple dose schedule. In a particular embodiment, said multiple dose schedule consists of a series of 2 doses separated by an interval of about 1 month to about 2 months. In a particular embodiment, said multiple dose schedule consists of a series of 2 doses separated by an interval of about 1 month, or a series of 2 doses separated by an interval of about 2 months.

In another embodiment, said multiple dose schedule consists of a series of 3 doses separated by an interval of about 1 month to about 2 months. In another embodiment, said multiple dose schedule consists of a series of 3 doses separated by an interval of about 1 month, or a series of 3 doses separated by an interval of about 2 months.

In another embodiment, said multiple dose schedule consists of a series of 3 doses separated by an interval of about 1 month to about 2 months followed by a fourth dose about 10 months to about 13 months after the first dose. In another embodiment, said multiple dose schedule consists of a series of 3 doses separated by an interval of about 1 month followed by a fourth dose about 10 months to about 13 months after the first dose, or a series of 3 doses separated by an interval of about 2 months followed by a fourth dose about 10 months to about 13 months after the first dose.

In an embodiment, the multiple dose schedule consists of at least one dose (e.g., 1, 2 or 3 doses) in the first year of age followed by at least one toddler dose.

In an embodiment, the multiple dose schedule consists of a series of 2 or 3 doses separated by an interval of about 1 month to about 2 months (for example 28-56 days between doses), starting at 2 months of age, and followed by a toddler dose at 12-18 months of age. In an embodiment, said multiple dose schedule consists of a series of 3 doses separated by an interval of about 1 to 2 months (for example 28-56 days between doses), starting at 2 months of age, and followed by a toddler dose at 12-15 months of age. In another embodiment, said multiple dose schedule consists of a series of 2 doses separated by an interval of about 2 months, starting at 2 months of age, and followed by a toddler dose at 12-18 months of age.

In an embodiment, the multiple dose schedule consists of a 4-dose series of vaccine at 2, 4, 6, and 12-15 months of age.

In an embodiment, a prime dose is given at day 0 and one or more boosts are given at intervals that range from about 2 to about 24 weeks, preferably with a dosing interval of 4-8 weeks.

In an embodiment, a prime dose is given at day 0 and a boost is given about 3 months later.

10. Immunization Schedule of the Set of Immunogenic Compositions of the Invention

In some cases, as little as one dose of the set of immunogenic compositions according to the invention is needed, but under some circumstances, such as conditions of greater immune deficiency or immune immaturity, a second, third or fourth dose may be given.

Following an initial vaccination, subjects can receive one or several booster immunizations adequately spaced.

In an embodiment, the schedule of vaccination of the set of immunogenic compositions according to the invention is a single dose. In a particular embodiment, said single dose schedule is for healthy persons being at least 2 years of age.

In an embodiment, the schedule of vaccination of the set of immunogenic compositions to the invention is a multiple dose schedule. A multiple dose schedule is frequently used in conditions such as immune deficiency (such as human elderly or human immunocompromised individuals) or immune immaturity (such as human newborns (i.e., under three months of age), infants (i.e., from 3 months to one year of age) or toddlers (i.e., from one year to four years of age)). In a particular embodiment, said multiple dose schedule consists of a series of 2 doses separated by an interval of about 1 month to about 12 months. In a particular embodiment, said multiple dose schedule consists of a series of 2 doses separated by an interval of about 1 month to about 6 months. In a particular embodiment, said multiple dose schedule consists of a series of 2 doses separated by an interval of about 1 month. In a particular embodiment, said multiple dose schedule consists of a series of 2 doses separated by an interval of about 2 months.

In another embodiment, said multiple dose schedule consists of a series of 3 doses wherein each dose is separated by an interval of about 1 month to about 12 months.

In a particular embodiment, said multiple dose schedule consists of a series of 3 doses wherein each dose is separated by an interval of about 1 month to about 6 months.

In another embodiment, said multiple dose schedule consists of a series of 4 doses wherein each dose is separated by an interval of about 1 month to about 12 months.

In a particular embodiment, said multiple dose schedule consists of a series of 4 doses wherein each dose is separated by an interval of about 1 month to about 6 months.

In another embodiment, said multiple dose schedule consists of a series of 3 doses wherein each dose is separated by an interval of about 1 month to about 4 months followed by a fourth dose about 10 months to about 13 months after the first dose.

In another embodiment, said multiple dose schedule consists of a series of 3 doses wherein each dose is separated by an interval of about 1 month to about 2 months followed by a fourth dose about 10 months to about 13 months after the first dose.

In an embodiment, the multiple dose schedule consists of at least one dose (e.g., 1, 2 or 3 doses) in the first year of age followed by at least one toddler dose.

In an embodiment, the multiple dose schedule consists of a series of 2 or 3 doses wherein each dose is separated by an interval of about 1 month to about 2 months (for example 28-56 days between doses), starting at 2 months of age, and followed by a toddler dose at 12-18 months of age. In an embodiment, said multiple dose schedule consists of a series of 3 doses wherein each dose is separated by an interval of about 1 month to about 2 months (for example 28-56 days between doses), starting at 2 months of age, and followed by a toddler dose at 12-15 months of age. In another embodiment, said multiple dose schedule consists of a series of 2 doses separated by an interval of about 2 months, starting at 2 months of age, and followed by a toddler dose at 12-18 months of age.

In an embodiment, the multiple dose schedule consists of a 4-dose series of vaccine at 2, 4, 6, and 12-15 months of age.

In an embodiment, a prime dose is given at day 0 and one or more booster doses are given at intervals that range from about 2 to about 24 weeks between doses, preferably with a dosing interval of 4-8 weeks.

In an embodiment, a prime dose is given at day 0 and a boost is given about 3 months later.

In another embodiment, said multiple dose schedule consists of a series of 5 doses wherein each dose is separated by an interval of about 1 month to about 12 months.

In a particular embodiment, said multiple dose schedule consists of a series of 5 doses wherein each dose is separated by an interval of about 1 month to about 6 months. In a particular embodiment, said multiple dose schedule consists of a series of 5 doses wherein each dose is separated by an interval of about 1, 2, 3, 4, 5 or 6 months. In a particular embodiment, said multiple dose schedule consists of a series of 5 doses wherein each dose is separated by an interval of about 1 month, or a series of 5 doses wherein each dose is separated by an interval of about 2 months.

An aspect of the invention pertains to any of the set of immunogenic compositions of the invention for simultaneous, concurrent, concomitant or sequential administration with a second immunogenic composition.

In an aspect of the present invention, the first immunogenic composition of the set of immunogenic compositions according to the invention is administered simultaneously, concurrently, concomitantly or sequentially with the second immunogenic composition.

In an aspect of the present invention, the first immunogenic composition of the set of immunogenic compositions according to the invention is administered simultaneously with the second immunogenic composition.

In an aspect of the present invention, the first immunogenic composition of the set of immunogenic compositions according to the invention is administered concurrently, with the second immunogenic composition.

In an aspect of the present invention, the first immunogenic composition of the set of immunogenic compositions according to the invention is administered concomitantly with the second immunogenic composition.

In an aspect of the present invention, the first immunogenic composition of the set of immunogenic compositions according to the invention is administered sequentially with the second immunogenic composition.

In some cases, as little as one dose of each of the immunogenic compositions is needed, but under some circumstances, a second, third or fourth dose of one or each of the immunogenic composition may be given. Following an initial vaccination, subjects can receive one or several booster immunizations adequately spaced.

In an embodiment, the present invention pertains a set of immunogenic compositions as disclosed therein for use in concomitant administration of the first and second immunogenic composition.

In an embodiment, the schedule of vaccination of said concomitant administration is a single dose (the administration of the first and second immunogenic composition, though in separate unit dosage forms, is considered as a single dose for purposes of defining the immunization schedule). In a particular embodiment, said single dose schedule is for healthy persons being at least 2 years of age.

In an embodiment, the schedule of vaccination of said concomitant administration is a multiple dose schedule (the administration of the first and second immunogenic composition, though in separate unit dosage forms, is considered as a single dose for purposes of defining the immunization schedule). In a particular embodiment, said multiple dose schedule consists of a series of 2 doses separated by an interval of about 1 month to about 12 months. In a particular embodiment, said schedule consists of a series of 2 doses separated by an interval of about 1 month to about 6 months. In a particular embodiment, said schedule consists of a series of 2 doses separated by an interval of about 1 month. In a particular embodiment, said schedule consists of a series of 2 doses separated by an interval of about 2 months.

In another embodiment, said multiple dose schedule consists of a series of 3 doses wherein each dose is separated by an interval of about 1 month to about 12 months. In a particular embodiment, said schedule consists of a series of 3 doses wherein each dose is separated by an interval of about 1 month to about 6 months. In another embodiment, said schedule consists of a series of 3 doses wherein each dose is separated by an interval of about 1 month. In another embodiment, said schedule consists of a series of 3 doses wherein each dose is separated by an interval of about 2 months.

In a particular embodiment, said multiple dose schedule consists of a series of 4 doses separated by an interval of about 1 month to about 12 months. In a particular embodiment, said multiple dose schedule consists of a series of 4 doses separated by an interval of about 1 month to about 6 months. In a particular embodiment, said multiple dose schedule consists of a series of 4 doses separated by an interval of about 1 month. In a particular embodiment, said multiple dose schedule consists of a series of 4 doses separated by an interval of about 2 months.

In another embodiment, said multiple dose schedule consists of a series of 3 doses wherein each dose is separated by an interval of about 1 month to about 4 months followed by a fourth dose about 10 months to about 13 months after the first dose. In another embodiment, said schedule consists of a series of 3 doses wherein each dose is separated by an interval of about 1 month to about 2 months followed by a fourth dose about 10 months to about 13 months after the first dose. In another embodiment, said schedule consists of a series of 3 doses wherein each dose is separated by an interval of about 1 month followed by a fourth dose about 10 months to about 13 months after the first dose. In another embodiment, said schedule consists of a series of 3 doses wherein each dose is separated by an interval of about 2 months followed by a fourth dose about 10 months to about 13 months after the first dose.

In an embodiment, the multiple dose schedule consists of at least one dose (e.g., 1, 2 or 3 doses) in the first year of age followed by at least one toddler dose.

In an embodiment, the multiple dose schedule consists of a series of 2 or 3 doses wherein each dose is separated by an interval of about 1 month to about 2 months (for example 28-56 days between doses), starting at 2 months of age, and followed by a toddler dose at 12-18 months of age. In an embodiment, said schedule consists of a series of 3 doses wherein each dose is separated by an interval of about 1 month to about 2 months (for example 28-56 days between doses), starting at 2 months of age, and followed by a toddler dose at 12-15 months of age. In another embodiment, said schedule consists of a series of 2 doses separated by an interval of about 2 months, starting at 2 months of age, and followed by a toddler dose at 12-18 months of age.

In an embodiment, the multiple dose schedule consists of a 4-dose series of vaccine administered at 2, 4, 6, and 12-15 months of age.

In an embodiment, a prime dose is given at day 0 and one or more booster doses are given at intervals that range from about 2 to about 24 weeks, preferably with a dosing interval of 4-8 weeks.

In an embodiment, a prime dose is given at day 0 and a boost is given about 3 months later.

In another embodiment, the present invention pertains a set of immunogenic compositions as disclosed therein for use in concurrent administration of the first and second immunogenic composition.

In an embodiment, the schedule of vaccination of said concurrent administration is a single dose (the administration of the first and second immunogenic composition, though in separate unit dosage forms, is considered as a single dose for purposes of defining the immunization schedule). In a particular embodiment, said single dose schedule is for healthy persons being at least 2 years of age.

In an embodiment, the schedule of vaccination of said concurrent administration is a multiple dose schedule, in particular any of the multiple schedules disclosed above for a concomitant administration.

In an embodiment, the present invention pertains to pertains a set of immunogenic compositions as disclosed therein for use in of the first and second immunogenic composition.

In an embodiment, the first immunogenic composition according to the invention is administered first and the second immunogenic composition is administered second. In another embodiment, the second immunogenic composition is administered first and the first immunogenic composition according to the invention is administered second.

In an embodiment, the schedule of vaccination of said sequential administration consists of a series of 2, 3, 4, 5, 6, 7 or 8 doses.

In an embodiment, the schedule of vaccination of said sequential administration consists of a series of 2 doses. In an embodiment, the schedule of vaccination of said sequential administration consists of a series of 4 doses. In an embodiment, the schedule of vaccination of said sequential administration consists of a series of 6 doses. In an embodiment, the schedule of vaccination of said sequential administration consists of a series of 8 doses.

In an embodiment, the schedule of vaccination of said sequential administration consists of a series of 2 doses. In an embodiment, the schedule of vaccination of said sequential administration consists of a series of 2 doses separated by an interval of about 1 month to about 12 months. In a particular embodiment, said multiple dose schedule consists of a series of 2 doses separated by an interval of about 1 month to about 6 months. In a particular embodiment, said multiple dose schedule consists of a series of 2 doses separated by an interval of about 1 month. In a particular embodiment, said multiple dose schedule consists of a series of 2 doses separated by an interval of about 2 months.

In an embodiment of said 2-dose schedule, the first immunogenic composition according to the invention is administered first and the second immunogenic composition is administered second. In another embodiment, the second immunogenic composition is administered first and the first immunogenic composition according to the invention is administered second.

In an embodiment, the schedule of vaccination of said sequential administration consists of a series of 3 doses. In a particular embodiment, said schedule consists of a series of 3 doses wherein each dose is separated by an interval of about 1 to about 12 months. In a particular embodiment, said schedule consists of a series of 3 doses wherein each dose is separated by an interval of about 1 month to about 6 months. In a particular embodiment, said schedule consists of a series of 3 doses wherein each dose is separated by an interval of about 1 to about 2 months.

In an embodiment of said 3-dose schedule, the first immunogenic composition according to the invention is administered as the first two doses and the second immunogenic composition is administered as the third dose.

In another embodiment of said 3-dose schedule, the second immunogenic composition is administered as the first two doses and the first immunogenic composition according to the invention is administered as the third dose.

In another embodiment of said 3-dose schedule, the first immunogenic composition according to the invention is administered as the first dose, the second immunogenic composition is administered as the second dose and the first immunogenic composition according to the invention is administered as the third dose.

In yet another embodiment of said 3-dose schedule, the second immunogenic composition is administered as the first dose, the first immunogenic composition according to the invention is administered as the second dose and the second immunogenic composition is administered as the third dose.

In yet another embodiment of said 3-dose schedule, the first immunogenic composition according to the invention is administered as the first dose and the second immunogenic composition is administered as the second and third doses.

In another embodiment of said 3-dose schedule, the second immunogenic composition is administered as the first dose and the first immunogenic composition according to the invention is administered as the second and third doses.

In an embodiment, the schedule of vaccination of said sequential administration consists of a series of 4 doses.

In a particular embodiment, said schedule consists of a series of 4 doses wherein each dose is separated by an interval of about 1 to about 12 months. In a particular embodiment, said schedule consists of a series of 4 doses wherein each dose is separated by an interval of about 1 month to about 6 months. In a particular embodiment, said schedule consists of a series of 4 doses wherein each dose is separated by an interval of about 1 to about 2 months.

In an embodiment of said 4-dose schedule, the first immunogenic composition according to the invention is administered as the first three doses and the second immunogenic composition is administered as the fourth dose.

In another embodiment of said 4-dose schedule, the second immunogenic composition is administered as the first three doses and the first immunogenic composition according to the invention is administered as the fourth dose.

In another embodiment of said 4-dose schedule, the first immunogenic composition according to the invention is administered as the first and second doses and the second immunogenic composition is administered as the third and fourth doses.

In another embodiment of said 4-dose schedule, the second immunogenic composition is administered as the first and second doses and the first immunogenic composition according to the invention is administered as the third and fourth doses.

In another embodiment of said 4-dose schedule, the first immunogenic composition according to the invention is administered as the first and second doses, the second immunogenic composition is administered as the third dose and the first immunogenic composition according to the invention is administered as the fourth dose.

In another embodiment of said 4-dose schedule, the second immunogenic composition is administered as the first and second doses, the first immunogenic composition according to the invention is administered as the third dose and the second immunogenic composition is administered as the fourth dose.

In another embodiment of said 4-dose schedule, the first immunogenic composition according to the invention is administered as the first dose and the second immunogenic composition is administered as the second, third and fourth doses.

In another embodiment of said 4-dose schedule, the second immunogenic composition is administered as the first dose and the first immunogenic composition according to the invention is administered as the second, third and fourth doses.

In another embodiment of said 4-dose schedule, the first immunogenic composition according to the invention is administered as the first dose, the second immunogenic composition is administered as the second dose, the first immunogenic composition according to the invention is administered as the third dose and the second immunogenic composition is administered as the fourth dose.

In another embodiment of said 4-dose schedule, the second immunogenic composition is administered as the first dose, the first immunogenic composition according to the invention is administered as the second dose, the second immunogenic composition is administered as the third dose and the first immunogenic composition according to the invention is administered as the fourth dose.

In another embodiment of said 4-dose schedule, the first immunogenic composition according to the invention is administered as the first dose, the second immunogenic composition is administered as the second dose and the first immunogenic composition according to the invention is administered as the third and fourth doses.

In another embodiment of said 4-dose schedule, the second immunogenic composition is administered as the first dose, the first immunogenic composition according to the invention is administered as the second dose and the second immunogenic composition is administered as the third and fourth doses.

In another embodiment of said 4-dose schedule, the first immunogenic composition according to the invention is administered as the first dose, the second immunogenic composition is administered as the second and third doses and the first immunogenic composition according to the invention is administered as the fourth dose.

In another embodiment of said 4-dose schedule, the second immunogenic composition is administered as the first dose, the first immunogenic composition according to the invention is administered as the second and third doses and the second immunogenic composition is administered as the fourth dose.

In an embodiment, the schedule of vaccination of said sequential administration consists of a series of 5 doses.

In a particular embodiment, said schedule consists of a series of 5 doses wherein each dose is separated by an interval of about 1 to about 12 months. In a particular embodiment, said schedule consists of a series of 5 doses wherein each dose is separated by an interval of about 1 month to about 6 months. In a particular embodiment, said schedule consists of a series of 5 doses wherein each dose is separated by an interval of about 1 to about 2 months.

In an embodiment of said 5-doses schedule, the first immunogenic composition (designated 1^{st} IC in the below table) and the second immunogenic composition (designated 2^{nd} IC in the below table) are administered in the following order:

| | Dose | | | | |
|---|---|---|---|---|---|
| Schedule number | 1 | 2 | 3 | 4 | 5 |
| 1 | 2^{nd} IC | 2^{nd} IC | 2^{nd} IC | 2^{nd} IC | 1^{st} IC |
| 2 | 2^{nd} IC | 2^{nd} IC | 2^{nd} IC | 1^{st} IC | 2^{nd} IC |
| 3 | 2^{nd} IC | 2^{nd} IC | 2^{nd} IC | 1^{st} IC | 1^{st} IC |
| 4 | 2^{nd} IC | 2^{nd} IC | 1^{st} IC | 2^{nd} IC | 2^{nd} IC |
| 5 | 2^{nd} IC | 2^{nd} IC | 1^{st} IC | 1^{st} IC | 2^{nd} IC |
| 6 | 2^{nd} IC | 2^{nd} IC | 1^{st} IC | 1^{st} IC | 1^{st} IC |
| 7 | 2^{nd} IC | 2^{nd} IC | 1^{st} IC | 2^{nd} IC | 1^{st} IC |
| 8 | 2^{nd} IC | 1^{st} IC | 2^{nd} IC | 2^{nd} IC | 2^{nd} IC |
| 9 | 2^{nd} IC | 1^{st} IC | 2^{nd} IC | 2^{nd} IC | 1^{st} IC |
| 10 | 2^{nd} IC | 1^{st} IC | 2^{nd} IC | 1^{st} IC | 2^{nd} IC |
| 11 | 2^{nd} IC | 1^{st} IC | 2^{nd} IC | 1^{st} IC | 1^{st} IC |
| 12 | 2^{nd} IC | 1^{st} IC | 1^{st} IC | 2^{nd} IC | 2^{nd} IC |
| 13 | 2^{nd} IC | 1^{st} IC | 1^{st} IC | 2^{nd} IC | 1^{st} IC |
| 14 | 2^{nd} IC | 1^{st} IC | 1^{st} IC | 1^{st} IC | 2^{nd} IC |
| 15 | 2^{nd} IC | 1^{st} IC | 1^{st} IC | 1^{st} IC | 1^{st} IC |
| 16 | 1^{st} IC | 2^{nd} IC | 2^{nd} IC | 2^{nd} IC | 2^{nd} IC |
| 17 | 1^{st} IC | 2^{nd} IC | 2^{nd} IC | 2^{nd} IC | 1^{st} IC |
| 18 | 1^{st} IC | 2^{nd} IC | 2^{nd} IC | 1^{st} IC | 2^{nd} IC |
| 19 | 1^{st} IC | 2^{nd} IC | 2^{nd} IC | 1^{st} IC | 1^{st} IC |
| 20 | 1^{st} IC | 2^{nd} IC | 1^{st} IC | 2^{nd} IC | 2^{nd} IC |
| 21 | 1^{st} IC | 2^{nd} IC | 1^{st} IC | 2^{nd} IC | 1^{st} IC |
| 22 | 1^{st} IC | 2^{nd} IC | 1^{st} IC | 1^{st} IC | 2^{nd} IC |
| 23 | 1^{st} IC | 2^{nd} IC | 1^{st} IC | 1^{st} IC | 1^{st} IC |
| 24 | 1^{st} IC | 1^{st} IC | 2^{nd} IC | 2^{nd} IC | 2^{nd} IC |
| 25 | 1^{st} IC | 1^{st} IC | 2^{nd} IC | 2^{nd} IC | 1^{st} IC |
| 26 | 1^{st} IC | 1^{st} IC | 2^{nd} IC | 1^{st} IC | 2^{nd} IC |
| 27 | 1^{st} IC | 1^{st} IC | 2^{nd} IC | 1^{st} IC | 1^{st} IC |
| 28 | 1^{st} IC | 1^{st} IC | 1^{st} IC | 2^{nd} IC | 2^{nd} IC |
| 29 | 1^{st} IC | 1^{st} IC | 1^{st} IC | 2^{nd} IC | 1^{st} IC |
| 30 | 1^{st} IC | 1^{st} IC | 1^{st} IC | 1^{st} IC | 2^{nd} IC |

The above table provide the order of administration of the first and second immunogenic composition (designated 1^{st} IC and 2^{nd} IC respectively) for the different doses, for example schedule number 1 is to be read as: in embodiment of said 5-dose schedule, the second immunogenic composition is administered as the first, second, third and fourth doses and the first immunogenic composition according to the invention is administered as the fifth dose.

In a preferred embodiment, the order of administration of the first and second immunogenic composition is according to schedule 1, 2, 3, 4, 5, 6, 7, 8, 9, 10, 11, 12, 13, 16, 17, 18, 19, 20 or 21.

In an embodiment, the schedule of vaccination of said sequential dose consists of a series of 6 doses.

In a particular embodiment, said schedule consists of a series of 6 doses wherein each dose is separated by an interval of about 1 to about 12 months. In a particular embodiment, said schedule consists of a series of 6 doses wherein each dose is separated by an interval of about 1 month to about 6 months. In a particular embodiment, said schedule consists of a series of 6 doses wherein each dose is separated by an interval of about 1 to about 2 months.

In an embodiment of said 6-doses schedule, the first immunogenic composition and the second immunogenic composition are administered in the order according to the any of the 30 schedules provided for the 5-doses schedule (see above table, schedule 1 to 30), followed by a sixth dose. In an embodiment, the first immunogenic composition according to the invention is administered as the sixth dose. In another embodiment, the second immunogenic composition is administered as the sixth dose.

In an embodiment, the schedule of vaccination of said sequential dose consists of a series of 7 doses.

In a particular embodiment, said schedule consists of a series of 7 doses wherein each dose is separated by an interval of about 1 to about 12 months. In a particular embodiment, said schedule consists of a series of 7 doses wherein each dose is separated by an interval of about 1 month to about 6 months. In a particular embodiment, said schedule consists of a series of 7 doses wherein each dose is separated by an interval of about 1 to about 2 months.

In an embodiment of said 7-doses schedule, the first immunogenic composition and the second immunogenic composition are administered in the order according to the any of the schedules provided for the 6-doses schedule (see above), followed by a seventh dose. In an embodiment, the first immunogenic composition according to the invention is administered as the seventh dose. In another embodiment, the second immunogenic composition is administered as the seventh dose.

In an embodiment, the schedule of vaccination of said sequential dose consists of a series of 8 doses.

In a particular embodiment, said schedule consists of a series of 8 doses wherein each dose is separated by an interval of about 1 to about 12 months. In a particular embodiment, said schedule consists of a series of 8 doses wherein each dose is separated by an interval of about 1 month to about 6 months. In a particular embodiment, said schedule consists of a series of 8 doses wherein each dose is separated by an interval of about 1 to about 2 months.

In an embodiment of said 8-doses schedule, the first immunogenic composition according to the invention and the second immunogenic composition are administered in the order according to the any of the schedules provided for the 7-doses schedule (see above), followed by an eighth dose. In an embodiment, the first immunogenic composition according to the invention is administered as the eighth dose. In another embodiment, the second immunogenic composition is administered as the eighth dose.

In an embodiment, the immunogenic compositions disclosed herein are administered by intramuscular injection. In an embodiment, the immunogenic compositions disclosed herein are administered by subcutaneous injection.

In an embodiment, the immunogenic compositions are administered by intramuscular injection in a thigh or arm. In an embodiment, the injection site is the anterolateral thigh muscle or the deltoid muscle.

In an embodiment, the immunogenic compositions are administered by subcutaneous injection in a thigh or an arm. In an embodiment, the injection site is the fatty tissue over the anterolateral thigh muscle or the fatty tissue over triceps.

In case of concomitant administration, the first injection can be made in one thigh and the second in the other thigh (preferably in the anterolateral thigh muscles). Alternatively, the first injection can be made in one arm and the second in the other arm (preferably in the deltoid muscles). The first injection can also be made in a thigh and the second in an arm or the first injection in an arm and the second in a thigh.

In an aspect the invention pertains to a set of immunogenic compositions of the present invention for use in any of the immunization schedules disclosed above.

11 Kit and Process

In an embodiment, the invention is directed toward a kit comprising an immunogenic composition or a set of immunogenic compositions disclosed herein and an information leaflet.

In an embodiment said information leaflet mentions the ability of the composition or set of immunogenic compositions to elicit functional antibodies against S. pneumoniae serotypes 15A, 15B and 15C.

In an embodiment said information leaflet mentions the ability of the composition or set of immunogenic compositions to elicit anti-capsular antibodies against S. pneumoniae serotypes 15A, 15B and 15C at a concentration≥0.35 μg/mL in a human population.

In an embodiment said information leaflet mentions the ability of the composition or set of immunogenic compositions to elicit OPA titers against S. pneumoniae serotypes 15A, 15B and 15C in a human population.

In an embodiment, the invention is directed toward a process for producing a kit comprising an immunogenic composition and an information leaflet, said process comprising the step of:
producing an immunogenic composition of the present disclosure and
combining in the same kit said immunogenic composition and information leaflet, wherein said information leaflet mentions the ability of said composition to elicit functional antibodies against S. pneumoniae serotypes 15A, 15B and 15C.

In an embodiment, the invention is directed toward a process for producing a kit comprising a set of immunogenic compositions and an information leaflet, said process comprising the step of:
producing a set of immunogenic compositions of the present disclosure and
combining in the same kit said set of immunogenic compositions and information leaflet, wherein said information leaflet mentions the ability of said set of immunogenic compositions to elicit functional antibodies against S. pneumoniae serotypes 15A, 15B and 15C.

In an embodiment, the invention is directed toward a process for producing a kit comprising an immunogenic composition and an information leaflet, said process comprising the step of:
producing an immunogenic composition of the present disclosure and
combining in the same kit said immunogenic composition and information leaflet, wherein said information leaflet mentions the ability of the composition to elicit anti-capsular antibodies against S. pneumoniae serotypes 15A, 15B and 15C at a concentration 0.35 μg/mL in a human population.

In an embodiment, the invention is directed toward a process for producing a kit comprising a set of immunogenic compositions and an information leaflet, said process comprising the step of:
producing a set of immunogenic compositions of the present disclosure and
combining in the same kit said set of immunogenic compositions and information leaflet, wherein said information leaflet mentions the ability of the set of immunogenic compositions to elicit anti-capsular antibodies against S. pneumoniae serotypes 15A, 15B and 15C at a concentration 0.35 μg/mL in a human population.

In an embodiment, the invention is directed toward a process for producing a kit comprising an immunogenic composition and an information leaflet, said process comprising the step of:
producing an immunogenic composition of the present disclosure and
combining in the same kit said immunogenic composition and information leaflet, wherein said information leaflet mentions the ability of the composition to elicit OPA titers against S. pneumoniae serotypes 15A, 15B and 15C in a human population.

In an embodiment, the invention is directed toward a process for producing a kit comprising a set of immunogenic compositions and an information leaflet, said process comprising the step of:
producing a set of immunogenic compositions of the present disclosure and
combining in the same kit said set of immunogenic compositions and information leaflet, wherein said information leaflet mentions the ability of the set of immunogenic compositions to elicit OPA titers against S. pneumoniae serotypes 15A, 15B and 15C in a human population.

In an embodiment, the invention is directed toward a process for producing a kit comprising an immunogenic composition and an information leaflet, said process comprising the step of:
producing an immunogenic composition of the present disclosure;
printing an information leaflet wherein said information leaflet mentions the ability of said composition to elicit functional antibodies against S. pneumoniae serotypes 15A, 15B and 15C;
combining in the same kit said immunogenic composition and said information leaflet.

In an embodiment, the invention is directed toward a process for producing a kit comprising a set of immunogenic compositions and an information leaflet, said process comprising the step of:
producing a set of immunogenic compositions of the present disclosure;
printing an information leaflet wherein said information leaflet mentions the ability of said set of immunogenic compositions to elicit functional antibodies against S. pneumoniae serotypes 15A, 15B and 15C;
combining in the same kit said set of immunogenic compositions and said information leaflet.

In an embodiment, the invention is directed toward a process for producing a kit comprising an immunogenic composition and an information leaflet, said process comprising the step of:
producing an immunogenic composition of the present disclosure;
printing an information leaflet wherein said information leaflet mentions the ability of the composition to elicit anti-capsular antibodies against S. pneumoniae serotypes 15A, 15B and 15C at a concentration 0.35 μg/mL in a human population;
combining in the same kit said immunogenic composition and said information leaflet.

In an embodiment, the invention is directed toward a process for producing a kit comprising a set of immunogenic compositions and an information leaflet, said process comprising the step of:
producing a set of immunogenic compositions of the present disclosure;
printing an information leaflet wherein said information leaflet mentions the ability of the set of immunogenic compositions to elicit anti-capsular antibodies against S. pneumoniae serotypes 15A, 15B and 15C at a concentration 0.35 μg/mL in a human population;
combining in the same kit said set of immunogenic compositions and said information leaflet.

In an embodiment, the invention is directed toward a process for producing a kit comprising an immunogenic composition and an information leaflet, said process comprising the step of:
producing an immunogenic composition of the present disclosure;
printing an information leaflet wherein said information leaflet mentions the ability of the composition to elicit OPA titers against S. pneumoniae serotypes 15A, 15B and 15C in a human population;
combining in the same kit said immunogenic composition and said information leaflet.

In an embodiment, the invention is directed toward a process for producing a kit comprising a set of immunogenic compositions and an information leaflet, said process comprising the step of:
producing a set of immunogenic compositions of the present disclosure;
printing an information leaflet wherein said information leaflet mentions the ability of the set of immunogenic compositions to elicit OPA titers against S. pneumoniae serotypes 15A, 15B and 15C in a human population;
combining in the same kit said set of immunogenic compositions and said information leaflet.

12 The Invention Also Provides the Following Embodiments as Defined in the Following Numbered Paragraphs 1 to 361
1. An immunogenic composition comprising a glycoconjugate from S. pneumoniae serotype 15B, a glycoconjugate from S. pneumoniae serotype 15C and a glycoconjugate from S. pneumoniae serotype 15A.
2. An immunogenic composition comprising a glycoconjugate from S. pneumoniae serotype 15B or 15C and a glycoconjugate from S. pneumoniae serotype 15A.
3. The immunogenic composition of paragraph 2 wherein said composition does not comprise capsular saccharide from S. pneumoniae serotype 15C when a glycoconjugate from S. pneumoniae serotype 15B is present in the composition and does not comprise capsular saccharide from S. pneumoniae serotype 15B when a glycoconjugate from S. pneumoniae serotype 15C is present.
4. An immunogenic composition comprising a glycoconjugate from S. pneumoniae serotype 15B and a glycoconjugate from S. pneumoniae serotype 15A.
5. The immunogenic composition of paragraph 4 wherein said composition does not comprise capsular saccharide from S. pneumoniae serotype 15C.
6. An immunogenic composition comprising a glycoconjugate from S. pneumoniae serotype 15C and a glycoconjugate from S. pneumoniae serotype 15A.
7. The immunogenic composition of paragraph 6 wherein said composition does not comprise capsular saccharide from S. pneumoniae serotype 15B.
8. The immunogenic composition of any one of paragraphs 1-7 wherein said immunogenic composition further comprises at least one glycoconjugate from S. pneumoniae serotypes 4, 6B, 9V, 14, 18C, 19F and/or 23F.
9. The immunogenic composition of any one of paragraphs 1-7 wherein said immunogenic composition further comprises at least one glycoconjugate from S. pneumoniae serotypes 4, 6B, 9V, 14, 18C, 19F and 23F.
10. The immunogenic composition of any one of paragraphs 1-7 wherein said immunogenic composition further comprises at least one glycoconjugate of each of the ten following S. pneumoniae serotypes: 1, 5, 4, 6B, 7F, 9V, 14, 18C, 19F and 23F.
11. The immunogenic composition of any one of paragraphs 1-7 wherein said immunogenic composition further comprises at least one glycoconjugate of each of the twelve following S. pneumoniae serotypes: 1, 4, 5, 6A, 6B, 7F, 9V, 14, 18C, 19A, 19F, and 23F.
12. The immunogenic composition of any one of paragraphs 1-7 wherein said immunogenic composition further comprises at least one glycoconjugate of each of the thirteen following S. pneumoniae serotypes: 1, 3, 4, 5, 6A, 6B, 7F, 9V, 14, 18C, 19A, 19F, and 23F.
13. The immunogenic composition of any one of paragraphs 1-12 wherein said immunogenic composition further comprises at least one glycoconjugate of S. pneumoniae serotype 22F.
14. The immunogenic composition of any one of paragraphs 1-13 wherein said immunogenic composition further comprises at least one glycoconjugate of S. pneumoniae serotype 33F.
15. The immunogenic composition of any one of paragraphs 1-14 wherein said immunogenic composition further comprises at least one glycoconjugate of S. pneumoniae serotype 8.
16. The immunogenic composition of any one of paragraphs 1-15 wherein said immunogenic composition further comprises at least one glycoconjugate of S. pneumoniae serotype 10A.
17. The immunogenic composition of any one of paragraphs 1-16 wherein said immunogenic composition further comprises at least one glycoconjugate of S. pneumoniae serotype 11A.
18. The immunogenic composition of any one of paragraphs 1-17 wherein said immunogenic composition further comprises at least one glycoconjugate of S. pneumoniae serotype 12F.
19. The immunogenic composition of any one of paragraphs 1-7 wherein said immunogenic composition further comprises at least one glycoconjugate of S. pneumoniae serotype 22F and at least one glycoconjugate of S. pneumoniae serotype 33F.
20. The immunogenic composition of any one of paragraphs 1-7 wherein said immunogenic composition further comprises at least one glycoconjugate of each of the six following S. pneumoniae serotypes: 22F, 33F, 12F, 10A, 11A and 8.
21. The immunogenic composition of any one of paragraphs 1-20 wherein all the glycoconjugates are individually conjugated to the carrier protein.
22. The immunogenic composition of any one of paragraphs 1-21 wherein if present the glycoconjugate from S. pneumoniae serotype 15B is conjugated to CRM₁₉₇.
23. The immunogenic composition of any one of paragraphs 1-22 wherein if present the glycoconjugate from S. pneumoniae serotype 15C is conjugated to CRM₁₉₇.
24. The immunogenic composition of any one of paragraphs 1-23 wherein if present, the glycoconjugate from S. pneumoniae serotype 15A is conjugated to CRM₁₉₇.
25. The immunogenic composition of any one of paragraphs 1-24 wherein if present, the glycoconjugate from S. pneumoniae serotype 22F is conjugated to CRM₁₉₇.
26. The immunogenic composition of any one of paragraphs 1-25 wherein if present, the glycoconjugate from S. pneumoniae serotype 33F is conjugated to CRM₁₉₇.
27. The immunogenic composition of any one of paragraphs 1-26 wherein if present, the glycoconjugate from S. pneumoniae serotype 12F is conjugated to CRM₁₉₇.
28. The immunogenic composition of any one of paragraphs 1-27 wherein if present, the glycoconjugate from S. pneumoniae serotype 10A is conjugated to CRM₁₉₇.
29. The immunogenic composition of any one of paragraphs 1-28 wherein if present, the glycoconjugate from S. pneumoniae serotype 11A is conjugated to CRM₁₉₇.
30. The immunogenic composition of any one of paragraphs 1-29 wherein if present, the glycoconjugate from S. pneumoniae serotype 8 is conjugated to CRM₁₉₇.
31. The immunogenic composition of any one of paragraphs 1-30 wherein if present, the glycoconjugates from S. pneumoniae serotypes 4, 6B, 9V, 14, 18C, 19F and 23F are conjugated to CRM₁₉₇.
32. The immunogenic composition of any one of paragraphs 1-31 wherein if present, the glycoconjugates from S. pneumoniae serotypes 1, 5 and 7F are conjugated to CRM₁₉₇.
33. The immunogenic composition of any one of paragraphs 1-32 wherein if present, the glycoconjugates from S. pneumoniae serotypes 6A and 19A are conjugated to CRM₁₉₇.
34. The immunogenic composition of any one of paragraphs 1-33 wherein if present, the glycoconjugate from S. pneumoniae serotype 3 is conjugated to CRM₁₉₇.
35. The immunogenic composition of any one of paragraphs 1-34 wherein all the glycoconjugates are individually conjugated to CRM₁₉₇.
36. The immunogenic composition of any one of paragraphs 1-24 wherein if present the glycoconjugates from S. pneumoniae serotypes 1, 4, 5, 6B, 7F, 9V, 14 and 23F are individually conjugated to PD, the glycoconjugate from S. pneumoniae serotype 18C is conjugated to TT and the glycoconjugate from S. pneumoniae serotype 19F is conjugated to DT.
37. The immunogenic composition of any one of paragraphs 1-36 comprising glycoconjugates from 8 different pneumococcal serotypes.
38. The immunogenic composition of any one of paragraphs 1-36 comprising glycoconjugates from 9 different pneumococcal serotypes.
39. The immunogenic composition of any one of paragraphs 1-36 comprising glycoconjugates from 10 different pneumococcal serotypes.
40. The immunogenic composition of any one of paragraphs 1-36 comprising glycoconjugates from 11 different pneumococcal serotypes.
41. The immunogenic composition of any one of paragraphs 1-36 comprising glycoconjugates from 12 different pneumococcal serotypes.
42. The immunogenic composition of any one of paragraphs 1-36 comprising glycoconjugates from 13 different pneumococcal serotypes.
43. The immunogenic composition of any one of paragraphs 1-36 comprising glycoconjugates from 14 different pneumococcal serotypes.
44. The immunogenic composition of any one of paragraphs 1-36 comprising glycoconjugates from 15 different pneumococcal serotypes.
45. The immunogenic composition of any one of paragraphs 1-36 comprising glycoconjugates from 16 different pneumococcal serotypes.
46. The immunogenic composition of any one of paragraphs 1-36 comprising glycoconjugates from 17 different pneumococcal serotypes.
47. The immunogenic composition of any one of paragraphs 1-36 comprising glycoconjugates from 18 different pneumococcal serotypes.
48. The immunogenic composition of any one of paragraphs 1-36 comprising glycoconjugates from 19 different pneumococcal serotypes.
49. The immunogenic composition of any one of paragraphs 1-36 comprising glycoconjugates from 20 different pneumococcal serotypes.
50. The immunogenic composition of any one of paragraphs 1-36 comprising glycoconjugates from 21 different pneumococcal serotypes.
51. The immunogenic composition of any one of paragraphs 1-36 comprising glycoconjugates from 22 different pneumococcal serotypes.
52. The immunogenic composition of any one of paragraphs 1-36 comprising glycoconjugates from 23 different pneumococcal serotypes.
53. The immunogenic composition of any one of paragraphs 1-36 comprising glycoconjugates from 24 different pneumococcal serotypes.
54. The immunogenic composition of any one of paragraphs 1-36 which is a 2, 3, 4, 5, 6, 7, 8, 9, 10, 11, 12, 13, 14, 15, 16, 17, 18, 19 or 20-valent pneumococcal conjugate composition.
55. The immunogenic composition of any one of paragraphs 1-36 which is a 16-valent pneumococcal conjugate composition.
56. The immunogenic composition of any one of paragraphs 1-36 which is a 19-valent pneumococcal conjugate composition.
57. The immunogenic composition of any one of paragraphs 1-36 which is a 20-valent pneumococcal conjugate composition.
58. The immunogenic composition of any one of paragraphs 1-36 which is a 21-valent pneumococcal conjugate composition.
59. The immunogenic composition of any one of paragraphs 1-36 comprising which is a 21-valent pneumococcal conjugate composition.
60. The immunogenic composition of any one of paragraphs 1-36 comprising which is a 22-valent pneumococcal conjugate composition.
61. The immunogenic composition of any one of paragraphs 1-36 comprising which is a 23-valent pneumococcal conjugate composition.
62. The immunogenic composition of any one of paragraphs 1-36 comprising which is a 24-valent pneumococcal conjugate composition.
63. The immunogenic composition of any one of paragraphs 1-63 wherein all the glycoconjugates of the immunogenic composition are individually conjugated to the carrier protein.
64. The immunogenic composition of any one of paragraphs 1-64 wherein the glycoconjugates are individually conjugated to CRM₁₉₇.
65. The immunogenic composition of any one of paragraphs 1-64 wherein the glycoconjugates are all individually conjugated to CRM₁₉₇.
66. A set of immunogenic compositions comprising: (a) a first immunogenic composition comprising a glycoconjugate from S. pneumoniae serotype 15C; and (b) a second immunogenic composition comprising a glycoconjugate from S. pneumoniae serotype 15A, wherein said compositions do not comprise capsular saccharide from S. pneumoniae serotypes 15B and wherein said immunogenic compositions are for simultaneous, concurrent, concomitant or sequential administration.
67. The set of immunogenic compositions of paragraph 66 for concurrent, concomitant or sequential administration.
68. A set of immunogenic compositions comprising: (a) a first immunogenic composition comprising a glycoconjugate from S. pneumoniae serotype 15B; and (b) a second immunogenic composition comprising a glycoconjugate from S. pneumoniae serotype 15A, wherein said compositions do not comprise capsular saccharide from S. pneumoniae serotypes 15C, wherein said compositions are for simultaneous, concurrent, concomitant or sequential administration.
69. The set of immunogenic compositions of paragraph 68 for concurrent, concomitant or sequential administration.
70. A set of immunogenic compositions comprising: (a) a first immunogenic composition comprising a glycoconjugate from S. pneumoniae serotype 15B; and (b) a second immunogenic composition comprising a glycoconjugate from S. pneumoniae serotype 15A and a glycoconjugate from S. pneumoniae serotype 15C, wherein said compositions are for simultaneous, concurrent, concomitant or sequential administration.
71. The set of immunogenic compositions of paragraph 70 for concurrent, concomitant or sequential administration.
72. A set of immunogenic compositions consisting of: (a) a first immunogenic composition comprising a glycoconjugate from S. pneumoniae serotype 15C; and (b) a second immunogenic composition comprising a glycoconjugate from S. pneumoniae serotype 15A, wherein said compositions do not comprise capsular saccharide from S. pneumoniae serotypes 15B and wherein said immunogenic compositions are for simultaneous, concurrent, concomitant or sequential administration.
73. The set of immunogenic compositions of paragraph 72 for concurrent, concomitant or sequential administration.
74. A set of immunogenic compositions consisting of: (a) a first immunogenic composition comprising a glycoconjugate from S. pneumoniae serotype 15B; and (b) a second immunogenic composition comprising a glycoconjugate from S. pneumoniae serotype 15A, wherein said compositions do not comprise capsular saccharide from S. pneumoniae serotypes 15C and wherein said compositions are for simultaneous, concurrent, concomitant or sequential administration.
75. The set of immunogenic compositions of paragraph 74 for concurrent, concomitant or sequential administration.
76. A set of immunogenic compositions consisting of: (a) a first immunogenic composition comprising a glycoconjugate from S. pneumoniae serotype 15B; and (b) a second immunogenic composition comprising a glycoconjugate from S. pneumoniae serotype 15A and a glycoconjugate from S. pneumoniae serotype 15C, wherein said immunogenic compositions are for simultaneous, concurrent, concomitant or sequential administration.
77. The set of immunogenic compositions of paragraph 76 for concurrent, concomitant or sequential administration.
78. The set of immunogenic compositions of any one of paragraphs 66-77 wherein said first immunogenic composition further comprises, in addition to a glycoconjugate from S. pneumoniae serotype 15B or 15C, glycoconjugates from S. pneumoniae serotypes 4, 6B, 9V, 14, 18C, 19F and 23F.
79. The set of immunogenic compositions of paragraphs 78 wherein said first immunogenic composition comprises in addition, glycoconjugates from S. pneumoniae serotypes 1, 5 and 7F.
80. The set of immunogenic compositions of any one of paragraphs 78-79 wherein said first immunogenic composition comprises in addition, glycoconjugates from S. pneumoniae serotypes 6A and 19A.
81. The set of immunogenic compositions of any one of paragraphs 78-80 wherein said first immunogenic composition comprises in addition, a glycoconjugate from S. pneumoniae serotype 3.
82. The set of immunogenic compositions of any one of paragraphs 78-81 wherein said first immunogenic composition comprises in addition, a glycoconjugate from S. pneumoniae serotype 22F.
83. The set of immunogenic compositions of any one of paragraphs 78-82 wherein said first immunogenic composition comprises in addition, a glycoconjugate from S. pneumoniae serotype 33F.
84. The set of immunogenic compositions of any one of paragraphs 78-81 wherein said first immunogenic composition further comprises, in addition to a glycoconjugate from S. pneumoniae serotype 15B or 15C, glycoconjugates from S. pneumoniae serotypes 8, 10A, 11A, 12F, 22F and 33F.
85. The set of immunogenic compositions of any one of paragraphs 78-84 wherein all the glycoconjugates of the first immunogenic compositions are individually conjugated to the carrier protein.
86. The set of immunogenic compositions of any one of paragraphs 78-85 wherein if present, the glycoconjugates from S. pneumoniae serotypes 4, 6B, 9V, 14, 18C, 19F and 23F of the first immunogenic composition are conjugated to CRM₁₉₇.
87. The set of immunogenic compositions of any one of paragraphs 78-86 wherein if present, the glycoconjugates from S. pneumoniae serotypes 1, 5 and 7F of the first immunogenic composition are conjugated to CRM₁₉₇.
88. The set of immunogenic compositions of any one of paragraphs 78-87 wherein if present, the glycoconjugates from S. pneumoniae serotypes 6A and 19A of the first immunogenic composition are conjugated to CRM₁₉₇.
89. The set of immunogenic compositions of any one of paragraphs 78-88 wherein if present, the glycoconjugate from S. pneumoniae serotype 3 of the first immunogenic composition is conjugated to CRM₁₉₇.
90. The set of immunogenic compositions of any one of paragraphs 78-89 wherein if present, the glycoconjugate from S. pneumoniae serotype 22F of the first immunogenic composition is conjugated to CRM₁₉₇.
91. The set of immunogenic compositions of any one of paragraphs 78-90 wherein if present, the glycoconjugate from S. pneumoniae serotype 33F of the first immunogenic composition is conjugated to CRM₁₉₇.
92. The set of immunogenic compositions of any one of paragraphs 78-91 wherein if present, the glycoconjugate from S. pneumoniae serotype 10A of the first immunogenic composition is conjugated to CRM₁₉₇.
93. The set of immunogenic compositions of any one of paragraphs 78-92 wherein if present, the glycoconjugate from S. pneumoniae serotype 11A of the first immunogenic composition is conjugated to CRM₁₉₇.
94. The set of immunogenic compositions of any one of paragraphs 78-93 wherein if present, the glycoconjugate from S. pneumoniae serotype 12F of the first immunogenic composition is conjugated to CRM₁₉₇.
95. The set of immunogenic compositions of any one of paragraphs 78-94 wherein if present, the glycoconjugate from S. pneumoniae serotype 8 of the first immunogenic composition is conjugated to CRM₁₉₇.
96. The set of immunogenic compositions of any one of paragraphs 78-95 wherein the glycoconjugates of the first immunogenic compositions are all individually conjugated to CRM₁₉₇.
97. The set of immunogenic compositions of any one of paragraphs 78-85 wherein if present, the glycoconjugates from S. pneumoniae serotypes 1, 4, 5, 6B, 7F, 9V, 14 and 23F of the first immunogenic composition are individually conjugated to PD, the glycoconjugate from S. pneumoniae serotype 18C of the first immunogenic composition is conjugated to TT and the glycoconjugate from S. pneumoniae serotype 19F of the first immunogenic composition is conjugated to DT.
98. The set of immunogenic compositions of any one of paragraphs 66-97 wherein the first immunogenic composition comprises from 7 to 25 different serotypes of S. pneumoniae.
99. The set of immunogenic compositions of any one of paragraphs 66-97 wherein the first immunogenic composition comprises glycoconjugates from 7 different serotypes.
100. The set of immunogenic compositions of any one of paragraphs 66-97 wherein the first immunogenic composition comprises glycoconjugates from 7 different serotypes.
101. The set of immunogenic compositions of any one of paragraphs 66-97 wherein the first immunogenic composition comprises glycoconjugates from 8 different serotypes.
102. The set of immunogenic compositions of any one of paragraphs 66-97 wherein the first immunogenic composition comprises glycoconjugates from 10 different serotypes.
103. The set of immunogenic compositions of any one of paragraphs 66-97 wherein the first immunogenic composition comprises glycoconjugates from 11 different serotypes.
104. The set of immunogenic compositions of any one of paragraphs 66-97 wherein the first immunogenic composition comprises glycoconjugates from 12 different serotypes.
105. The set of immunogenic compositions of any one of paragraphs 66-97 wherein the first immunogenic composition comprises glycoconjugates from 13 different serotypes.
106. The set of immunogenic compositions of any one of paragraphs 66-97 wherein the first immunogenic composition comprises glycoconjugates from 14 different serotypes.
107. The set of immunogenic compositions of any one of paragraphs 66-97 wherein the first immunogenic composition comprises glycoconjugates from 15 different serotypes.
108. The set of immunogenic compositions of any one of paragraphs 66-97 wherein the first immunogenic composition comprises glycoconjugates from 16 different serotypes.
109. The set of immunogenic compositions of any one of paragraphs 66-97 wherein the first immunogenic composition comprises glycoconjugates from 17 different serotypes.
110. The set of immunogenic compositions of any one of paragraphs 66-97 wherein the first immunogenic composition comprises glycoconjugates from 18 different serotypes.
111. The set of immunogenic compositions of any one of paragraphs 66-97 wherein the first immunogenic composition comprises glycoconjugates from 19 different serotypes.
112. The set of immunogenic compositions of any one of paragraphs 66-97 wherein the first immunogenic composition comprises glycoconjugates from 20 different serotypes.
113. The set of immunogenic compositions of any one of paragraphs 66-97 wherein the first immunogenic composition comprises glycoconjugates from 21 different serotypes.
114. The set of immunogenic compositions of any one of paragraphs 66-97 wherein the first immunogenic composition comprises glycoconjugates from 22 different serotypes.
115. The set of immunogenic compositions of any one of paragraphs 66-97 wherein the first immunogenic composition comprises glycoconjugates from 23 different serotypes.
116. The set of immunogenic compositions of any one of paragraphs 66-97 wherein the first immunogenic composition comprises glycoconjugates from 24 different serotypes.
117. The set of immunogenic compositions of any one of paragraphs 66-97 wherein the first immunogenic composition is a 7-valent pneumococcal conjugate composition.
118. The set of immunogenic compositions of any one of paragraphs 66-97 wherein the first immunogenic composition is an 8-valent pneumococcal conjugate composition.
119. The set of immunogenic compositions of any one of paragraphs 66-97 wherein the first immunogenic composition is a 9-valent pneumococcal conjugate composition.
120. The set of immunogenic compositions of any one of paragraphs 66-97 wherein the first immunogenic composition is a 10-valent pneumococcal conjugate composition.
121. The set of immunogenic compositions of any one of paragraphs 66-97 wherein the first immunogenic composition is a 11-valent pneumococcal conjugate composition.
122. The set of immunogenic compositions of any one of paragraphs 66-97 wherein the first immunogenic composition is a 12-valent pneumococcal conjugate composition.
123. The set of immunogenic compositions of any one of paragraphs 66-97 wherein the first immunogenic composition is a 13-valent pneumococcal conjugate composition.
124. The set of immunogenic compositions of any one of paragraphs 66-97 wherein the first immunogenic composition is a 14-valent pneumococcal conjugate composition.
125. The set of immunogenic compositions of any one of paragraphs 66-97 wherein the first immunogenic composition is a 15-valent pneumococcal conjugate composition.
126. The set of immunogenic compositions of any one of paragraphs 66-97 wherein the first immunogenic composition is a 16-valent pneumococcal conjugate composition.
127. The set of immunogenic compositions of any one of paragraphs 66-97 wherein the first immunogenic composition is a 17-valent pneumococcal conjugate composition.
128. The set of immunogenic compositions of any one of paragraphs 66-97 wherein the first immunogenic composition is an 18-valent pneumococcal conjugate composition.
129. The set of immunogenic compositions of any one of paragraphs 66-97 wherein the first immunogenic composition is a 19-valent pneumococcal conjugate composition.
130. The set of immunogenic compositions of any one of paragraphs 66-97 wherein the first immunogenic composition is a 20-valent pneumococcal conjugate composition.
131. The set of immunogenic compositions of any one of paragraphs 66-97 wherein the first immunogenic composition is a 21-valent pneumococcal conjugate composition.
132. The set of immunogenic compositions of any one of paragraphs 66-97 wherein the first immunogenic composition is a 22-valent pneumococcal conjugate composition.
133. The set of immunogenic compositions of any one of paragraphs 66-97 wherein the first immunogenic composition is a 23-valent pneumococcal conjugate composition.
134. The set of immunogenic compositions of any one of paragraphs 66-97 wherein the first immunogenic composition is a 24-valent pneumococcal conjugate composition.
135. The set of immunogenic compositions of any one of paragraphs 66-97 wherein the first immunogenic composition is a 25-valent pneumococcal conjugate composition.
136. The set of immunogenic compositions of any one of paragraphs 66-85 wherein, the first immunogenic composition is a 8-valent pneumococcal conjugate composition wherein in addition to a glycoconjugate from S. pneumoniae serotype 15B or 15C, said composition further comprises, glycoconjugates from S. pneumoniae serotypes 4, 6B, 9V, 14, 18C, 19F and 23F individually conjugated to CRM₁₉₇.
137. The set of immunogenic compositions of any one of paragraphs 66-85 wherein, the first immunogenic composition is a 14-valent pneumococcal conjugate composition wherein in addition to a glycoconjugate from S. pneumoniae serotype 15B or 15C, said composition further comprises, glycoconjugates from S. pneumoniae serotypes 1, 3, 4, 5, 6A, 6B, 7F, 9V, 14, 18C, 19A, 19F and 23F individually conjugated to CRM₁₉₇.
138. The set of immunogenic compositions of any one of paragraphs 66-85 wherein, the first immunogenic composition is a 15-valent pneumococcal conjugate composition wherein in addition to a glycoconjugate from S. pneumoniae serotype 15B or 15C, said composition further comprises, glycoconjugates from S. pneumoniae serotypes 1, 3, 4, 5, 6A, 6B, 7F, 9V, 14, 18C, 19A, 19F, 23F and 22F individually conjugated to CRM₁₉₇.
139. The set of immunogenic compositions of any one of paragraphs 66-85 wherein, the first immunogenic composition is a 15-valent pneumococcal conjugate composition wherein in addition to a glycoconjugate from S. pneumoniae serotype 15B or 15C, said composition further comprises, glycoconjugates from S. pneumoniae serotypes 1, 3, 4, 5, 6A, 6B, 7F, 9V, 14, 18C, 19A, 19F, 23F and 33F individually conjugated to CRM₁₉₇.
140. The set of immunogenic compositions of any one of paragraphs 66-85 wherein, the first immunogenic composition is a 16-valent pneumococcal conjugate composition wherein in addition to a glycoconjugate from S. pneumoniae serotype 15B or 15C, said composition further comprises, glycoconjugates from S. pneumoniae serotypes 1, 3, 4, 5, 6A, 6B, 7F, 9V, 14, 18C, 19A, 19F, 23F, 22F and 33F individually conjugated to CRM₁₉₇.
141. The set of immunogenic compositions of any one of paragraphs 66-85 wherein, the first immunogenic composition is a 20-valent pneumococcal conjugate composition wherein in addition to a glycoconjugate from S. pneumoniae serotype 15B or 15C, said composition further comprises, glycoconjugates from S. pneumoniae serotypes 1, 3, 4, 5, 6A, 6B, 7F, 8, 9V, 10A, 11A, 12F, 14, 18C, 19A, 19F, 23F, 22F and 33F individually conjugated to CRM₁₉₇.
142. The set of immunogenic compositions of any one of paragraphs 66-141 wherein, the first immunogenic compositions further comprise one or more adjuvants as disclosed at section 4.
143. The set of immunogenic compositions of any one of paragraphs 66-142 wherein the first immunogenic composition is formulated as disclosed at section 5.
144. The set of immunogenic compositions of any one of paragraphs 66-143 wherein said second immunogenic composition further comprises, in addition to a glycoconjugate from S. pneumoniae serotype 15A, glycoconjugates from S. pneumoniae serotypes 4, 6B, 9V, 14, 18C, 19F and 23F.
145. The set of immunogenic compositions of paragraph 144 wherein said second immunogenic composition comprises in addition glycoconjugates from S. pneumoniae serotypes 1, 5 and 7F.
146. The set of immunogenic compositions of any one of paragraphs 144-145 wherein said second immunogenic composition comprises in addition glycoconjugates from S. pneumoniae serotypes 6A and 19A.
147. The set of immunogenic compositions of any one of paragraphs 144-146 wherein said second immunogenic composition comprises in addition a glycoconjugate from S. pneumoniae serotype 3.
148. The set of immunogenic compositions of any one of paragraphs 144-147 wherein said second immunogenic composition comprises in addition a glycoconjugate from S. pneumoniae serotype 22F.
149. The set of immunogenic compositions of any one of paragraphs 144-148 wherein said second immunogenic composition comprises in addition a glycoconjugate from S. pneumoniae serotype 33F.
150. The set of immunogenic compositions of any one of paragraphs 66-149 wherein all the glycoconjugates of the second immunogenic composition are individually conjugated to the carrier protein.
151. The set of immunogenic compositions of any one of paragraphs 66-150 wherein if present, the glycoconjugates from S. pneumoniae serotypes 4, 6B, 9V, 14, 18C, 19F and 23F of the second immunogenic composition are conjugated to CRM₁₉₇.
152. The set of immunogenic compositions of any one of paragraphs 66-151 wherein if present, the glycoconjugates from S. pneumoniae serotypes 1, 5 and 7F of the second immunogenic composition are conjugated to CRM₁₉₇.
153. The set of immunogenic compositions of any one of paragraphs 66-152 wherein if present, the glycoconjugates from S. pneumoniae serotypes 6A and 19A of the second immunogenic composition are conjugated to CRM₁₉₇.
154. The set of immunogenic compositions of any one of paragraphs 66-153 wherein if present, the glycoconjugate from S. pneumoniae serotype 3 of the second immunogenic composition is conjugated to CRM₁₉₇.
155. The set of immunogenic compositions of any one of paragraphs 66-154 wherein if present, the glycoconjugate from S. pneumoniae serotype 22F of the second immunogenic composition is conjugated to CRM₁₉₇.
156. The set of immunogenic compositions of any one of paragraphs 66-155 wherein if present, the glycoconjugate from S. pneumoniae serotype 33F of the second immunogenic composition is conjugated to CRM₁₉₇.
157. The set of immunogenic compositions of any one of paragraphs 66-156 wherein if present, the glycoconjugate from S. pneumoniae serotype 10A of the second immunogenic composition is conjugated to CRM₁₉₇.
158. The set of immunogenic compositions of any one of paragraphs 66-157 wherein if present, the glycoconjugate from S. pneumoniae serotype 11A of the second immunogenic composition is conjugated to CRM₁₉₇.
159. The set of immunogenic compositions of any one of paragraphs 66-158 wherein if present, the glycoconjugate from S. pneumoniae serotype 12F of the second immunogenic composition is conjugated to CRM₁₉₇.
160. The set of immunogenic compositions of any one of paragraphs 66-153 wherein if present, the glycoconjugate from S. pneumoniae serotype 8 of the second immunogenic composition is conjugated to CRM₁₉₇.
161. The set of immunogenic compositions of any one of paragraphs 66-160 wherein the glycoconjugates of the second immunogenic compositions are all individually conjugated to CRM₁₉₇.
162. The set of immunogenic compositions of any one of paragraphs 66-150 wherein if present, the glycoconjugates from S. pneumoniae serotypes 1, 4, 5, 6B, 7F, 9V, 14 and 23F of the second immunogenic composition are individually conjugated to PD, the glycoconjugate from S. pneumoniae serotype 18C of the second immunogenic composition is conjugated to TT and the glycoconjugate from S. pneumoniae serotype 19F of the second immunogenic composition is conjugated to DT.
163. The set of immunogenic compositions of any one of paragraphs 66-162 wherein the second immunogenic composition comprises from 7 to 25 different serotypes of S. pneumoniae.
164. The set of immunogenic compositions of any one of paragraphs 66-162 wherein the second immunogenic composition comprises from 7 to 20 different serotypes of S. pneumoniae.
165. The set of immunogenic compositions of any one of paragraphs 66-162 wherein the second immunogenic compositions comprise glycoconjugates from 7 different serotypes.
166. The set of immunogenic compositions of any one of paragraphs 66-162 wherein the second immunogenic compositions comprise glycoconjugates from 8 different serotypes.
167. The set of immunogenic compositions of any one of paragraphs 66-162 wherein the second immunogenic compositions comprise glycoconjugates from 10 different serotypes.
168. The set of immunogenic compositions of any one of paragraphs 66-162 wherein the second immunogenic compositions comprise glycoconjugates from 15 different serotypes.
169. The set of immunogenic compositions of any one of paragraphs 66-162 wherein the second immunogenic compositions comprise glycoconjugates from 16 different serotypes.
170. The set of immunogenic compositions of any one of paragraphs 66-162 wherein the second immunogenic compositions comprise glycoconjugates from 20 different serotypes.
171. The set of immunogenic compositions of any one of paragraphs 66-162 wherein the second immunogenic composition is a 7, 8, 9, 10, 11, 12, 13, 14, 15, 16, 17, 18, 19, 20, 21, 22, 23, 24 or 25-valent pneumococcal conjugate composition.
172. The set of immunogenic compositions of any one of paragraphs 66-162 wherein the second immunogenic composition is a 7-valent pneumococcal conjugate composition.
173. The set of immunogenic compositions of any one of paragraphs 66-162 wherein the second immunogenic composition is an 8-valent pneumococcal conjugate composition.
174. The set of immunogenic compositions of any one of paragraphs 66-162 wherein the second immunogenic composition is a 9-valent pneumococcal conjugate composition.
175. The set of immunogenic compositions of any one of paragraphs 66-162 wherein the second immunogenic composition is a 10-valent pneumococcal conjugate composition.
176. The set of immunogenic compositions of any one of paragraphs 66-162 wherein the second immunogenic composition is an 11-valent pneumococcal conjugate composition.
177. The set of immunogenic compositions of any one of paragraphs 66-162 wherein the second immunogenic composition is a 12-valent pneumococcal conjugate composition.
178. The set of immunogenic compositions of any one of paragraphs 66-162 wherein the second immunogenic composition is a 13-valent pneumococcal conjugate composition.
179. The set of immunogenic compositions of any one of paragraphs 66-162 wherein the second immunogenic composition is a 14-valent pneumococcal conjugate composition.
180. The set of immunogenic compositions of any one of paragraphs 66-162 wherein the second immunogenic composition is a 15-valent pneumococcal conjugate composition.
181. The set of immunogenic compositions of any one of paragraphs 66-162 wherein the second immunogenic composition is a 16-valent pneumococcal conjugate composition.
182. The set of immunogenic compositions of any one of paragraphs 66-162 wherein the second immunogenic composition is a 17-valent pneumococcal conjugate composition.
183. The set of immunogenic compositions of any one of paragraphs 66-162 wherein the second immunogenic composition is an 18-valent pneumococcal conjugate composition.
184. The set of immunogenic compositions of any one of paragraphs 66-162 wherein the second immunogenic composition is a 19-valent pneumococcal conjugate composition.
185. The set of immunogenic compositions of any one of paragraphs 66-162 wherein the second immunogenic composition is a 20-valent pneumococcal conjugate composition.
186. The set of immunogenic compositions of any one of paragraphs 66-162 wherein the second immunogenic composition is a 21-valent pneumococcal conjugate composition.
187. The set of immunogenic compositions of any one of paragraphs 66-162 wherein the second immunogenic composition is a 22-valent pneumococcal conjugate composition.
188. The set of immunogenic compositions of any one of paragraphs 66-162 wherein the second immunogenic composition is a 23-valent pneumococcal conjugate composition.
189. The set of immunogenic compositions of any one of paragraphs 66-162 wherein the second immunogenic composition is a 24-valent pneumococcal conjugate composition.
190. The set of immunogenic compositions of any one of paragraphs 66-162 wherein the second immunogenic composition is a 25-valent pneumococcal conjugate composition.
191. The set of immunogenic compositions of any one of paragraphs 66-143 wherein, the second immunogenic composition is a 8-valent pneumococcal conjugate composition wherein in addition to a glycoconjugate from S. pneumoniae serotype 15A, said composition further comprises, glycoconjugates from S. pneumoniae serotypes 4, 6B, 9V, 14, 18C, 19F and 23F individually conjugated to CRM₁₉₇.
192. The set of immunogenic compositions of any one of paragraphs 66-143 wherein, the second immunogenic composition is a 14-valent pneumococcal conjugate composition wherein in addition to a glycoconjugate from S. pneumoniae serotype 15A, said composition further comprises, glycoconjugates from S. pneumoniae serotypes 1, 3, 4, 5, 6A, 6B, 7F, 9V, 14, 18C, 19A, 19F and 23F individually conjugated to CRM₁₉₇.
193. The set of immunogenic compositions of any one of paragraphs 66-143 wherein, the second immunogenic composition is a 15-valent pneumococcal conjugate composition wherein in addition to a glycoconjugate from S. pneumoniae serotype 15A, said composition further comprises, glycoconjugates from S. pneumoniae serotypes 1, 3, 4, 5, 6A, 6B, 7F, 9V, 14, 18C, 19A, 19F, 23F and 22F individually conjugated to CRM₁₉₇.
194. The set of immunogenic compositions of any one of paragraphs 66-143 wherein, the second immunogenic composition is a 15-valent pneumococcal conjugate composition wherein in addition to a glycoconjugate from S. pneumoniae serotype 15A, said composition further comprises, glycoconjugates from S. pneumoniae serotypes 1, 3, 4, 5, 6A, 6B, 7F, 9V, 14, 18C, 19A, 19F, 23F and 33F individually conjugated to CRM₁₉₇.
195. The set of immunogenic compositions of any one of paragraphs 66-143 wherein, the second immunogenic composition is a 16-valent pneumococcal conjugate composition wherein in addition to a glycoconjugate from S. pneumoniae serotype 15A, said composition further comprises, glycoconjugates from S. pneumoniae serotypes 1, 3, 4, 5, 6A, 6B, 7F, 9V, 14, 18C, 19A, 19F, 23F, 22F and 33F individually conjugated to CRM₁₉₇.
196. The set of immunogenic compositions of any one of paragraphs 66-143 wherein, the second immunogenic composition is a 20-valent pneumococcal conjugate composition wherein in addition to a glycoconjugate from S. pneumoniae serotype 15A, said composition further comprises, glycoconjugates from S. pneumoniae serotypes 1, 3, 4, 5, 6A, 6B, 7F, 8, 9V, 10A, 11A, 12F, 14, 18C, 19A, 19F, 23F, 22F and 33F individually conjugated to CRM₁₉₇.
197. The set of immunogenic compositions of any one of paragraphs 66-143 wherein, the second immunogenic composition is a 9-valent pneumococcal conjugate composition wherein in addition to a glycoconjugate from S. pneumoniae serotype 15A and a glycoconjugate from S. pneumoniae serotype 15C, said composition further comprises, glycoconjugates from S. pneumoniae serotypes 4, 6B, 9V, 14, 18C, 19F and 23F individually conjugated to CRM₁₉₇.
198. The set of immunogenic compositions of any one of paragraphs 66-143 wherein, the second immunogenic composition is a 15-valent pneumococcal conjugate composition wherein in addition to a glycoconjugate from S. pneumoniae serotype 15A and a glycoconjugate from S. pneumoniae serotype 15C, said composition further comprises, glycoconjugates from S. pneumoniae serotypes 1, 3, 4, 5, 6A, 6B, 7F, 9V, 14, 18C, 19A, 19F and 23F individually conjugated to CRM₁₉₇.
199. The set of immunogenic compositions of any one of paragraphs 66-143 wherein, the second immunogenic composition is a 16-valent pneumococcal conjugate composition wherein in addition to a glycoconjugate from S. pneumoniae serotype 15A and a glycoconjugate from S. pneumoniae serotype 15C, said composition further comprises, glycoconjugates from S. pneumoniae serotypes 1, 3, 4, 5, 6A, 6B, 7F, 9V, 14, 18C, 19A, 19F, 23F and 22F individually conjugated to CRM₁₉₇.
200. The set of immunogenic compositions of any one of paragraphs 66-143 wherein, the second immunogenic composition is a 16-valent pneumococcal conjugate composition wherein in addition to a glycoconjugate from S. pneumoniae serotype 15A and a glycoconjugate from S. pneumoniae serotype 15C, said composition further comprises, glycoconjugates from S. pneumoniae serotypes 1, 3, 4, 5, 6A, 6B, 7F, 9V, 14, 18C, 19A, 19F, 23F and 33F individually conjugated to CRM₁₉₇.
201. The set of immunogenic compositions of any one of paragraphs 66-143 wherein, the second immunogenic composition is a 17-valent pneumococcal conjugate composition wherein in addition to a glycoconjugate from S. pneumoniae serotype 15A and a glycoconjugate from S. pneumoniae serotype 15C, said composition further comprises, glycoconjugates from S. pneumoniae serotypes 1, 3, 4, 5, 6A, 6B, 7F, 9V, 14, 18C, 19A, 19F, 23F, 22F and 33F individually conjugated to CRM₁₉₇.
202. The set of immunogenic compositions of any one of paragraphs 66-143 wherein, the second immunogenic composition is a 21-valent pneumococcal conjugate composition wherein in addition to a glycoconjugate from S. pneumoniae serotype 15A and a glycoconjugate from S. pneumoniae serotype 15C, said composition further comprises, glycoconjugates from S. pneumoniae serotypes 1, 3, 4, 5, 6A, 6B, 7F, 8, 9V, 10A, 11A, 12F, 14, 18C, 19A, 19F, 23F, 22F and 33F individually conjugated to CRM₁₉₇.
203. The set of immunogenic compositions of any one of paragraphs 66-202 wherein, the second immunogenic compositions further comprise one or more adjuvants as disclosed at section 4.
204. The set of immunogenic compositions of any one of paragraphs 66-203 wherein, the second immunogenic composition is formulated as disclosed at section 5.
205. An immunogenic composition comprising a glycoconjugate from S. pneumoniae serotype 15A wherein said immunogenic composition does not comprise glycoconjugates from S. pneumoniae serotypes 15B and 15C.
206. An immunogenic composition comprising a glycoconjugate from S. pneumoniae serotype 15B wherein said immunogenic composition does not comprise glycoconjugates from S. pneumoniae serotypes 15A and 15C.
207. An immunogenic composition comprising a glycoconjugate from S. pneumoniae serotype 15C wherein said immunogenic composition does not comprise glycoconjugates from S. pneumoniae serotypes 15A and 15B.
208. An immunogenic composition comprising a glycoconjugate from S. pneumoniae serotype 15A and a glycoconjugate from S. pneumoniae serotype 15C wherein said immunogenic composition does not comprise a glycoconjugate from S. pneumoniae serotypes 15B.
209. An immunogenic composition comprising a glycoconjugate from S. pneumoniae serotype 15A and a glycoconjugate from S. pneumoniae serotype 15B wherein said immunogenic composition does not comprise a glycoconjugate from S. pneumoniae serotypes 15C.
210. An immunogenic composition comprising a glycoconjugate from S. pneumoniae serotype 15B and a glycoconjugate from S. pneumoniae serotype 15C wherein said immunogenic composition does not comprise a glycoconjugate from S. pneumoniae serotypes 15A.
211. An immunogenic composition comprising a glycoconjugate from S. pneumoniae serotype 15A wherein said immunogenic composition does not comprise glycoconjugates from S. pneumoniae serotypes 4, 6B, 9V, 14, 15B, 15C, 18C, 19F and 23F.
212. An immunogenic composition comprising a glycoconjugate from S. pneumoniae serotype 15B wherein said immunogenic composition does not comprise glycoconjugates from S. pneumoniae serotypes 4, 6B, 9V, 14, 15A, 15C, 18C, 19F and 23F.
213. An immunogenic composition comprising a glycoconjugate from S. pneumoniae serotype 15C wherein said immunogenic composition does not comprise glycoconjugates from S. pneumoniae serotypes 4, 6B, 9V, 14, 15A, 15B, 18C, 19F and 23F.
214. An immunogenic composition comprising a glycoconjugate from S. pneumoniae serotype 15A and a glycoconjugate from S. pneumoniae serotype 15C wherein said immunogenic composition does not comprise glycoconjugates from S. pneumoniae serotypes 4, 6B, 9V, 14, 15B, 18C, 19F and 23F.
215. An immunogenic composition comprising a glycoconjugate from S. pneumoniae serotype 15A and a glycoconjugate from S. pneumoniae serotype 15B wherein said immunogenic composition does not comprise glycoconjugates from S. pneumoniae serotypes 4, 6B, 9V, 14, 15C, 18C, 19F and 23F.
216. An immunogenic composition comprising a glycoconjugate from S. pneumoniae serotype 15B and a glycoconjugate from S. pneumoniae serotype 15C wherein said immunogenic composition does not comprise glycoconjugates from S. pneumoniae serotypes 4, 6B, 9V, 14, 15A, 18C, 19F and 23F.
217. An immunogenic composition comprising a glycoconjugate from S. pneumoniae serotype 15A wherein said immunogenic composition does not comprise glycoconjugates from S. pneumoniae serotypes 1, 4, 5, 6B, 7F, 9V, 14, 15B, 15C, 18C, 19F and 23F.
218. An immunogenic composition comprising a glycoconjugate from S. pneumoniae serotype 15B wherein said immunogenic composition does not comprise glycoconjugates from S. pneumoniae serotypes 1, 4, 5, 6B, 7F, 9V, 14, 15A, 15C, 18C, 19F and 23F.
219. An immunogenic composition comprising a glycoconjugate from S. pneumoniae serotype 15C wherein said immunogenic composition does not comprise glycoconjugates from S. pneumoniae serotypes 1, 4, 5, 6B, 7F, 9V, 14, 15A, 15B, 18C, 19F and 23F.
220. An immunogenic composition comprising a glycoconjugate from S. pneumoniae serotype 15A and a glycoconjugate from S. pneumoniae serotype 15C wherein said immunogenic composition does not comprise glycoconjugates from S. pneumoniae serotypes 1, 4, 5, 6B, 7F, 9V, 14, 15B, 18C, 19F and 23F.
221. An immunogenic composition comprising a glycoconjugate from S. pneumoniae serotype 15A and a glycoconjugate from S. pneumoniae serotype 15B wherein said immunogenic composition does not comprise glycoconjugates from S. pneumoniae serotypes 1, 4, 5, 6B, 7F, 9V, 14, 15C, 18C, 19F and 23F.
222. An immunogenic composition comprising a glycoconjugate from S. pneumoniae serotype 15B and a glycoconjugate from S. pneumoniae serotype 15C wherein said immunogenic composition does not comprise glycoconjugates from S. pneumoniae serotypes 1, 4, 5, 6B, 7F, 9V, 14, 15A, 18C, 19F and 23F.
223. An immunogenic composition comprising a glycoconjugate from S. pneumoniae serotype 15A wherein said immunogenic composition does not comprise glycoconjugates from S. pneumoniae serotypes 1, 4, 5, 6A, 6B, 7F, 9V, 14, 15B, 15C, 18C, 19A, 19F and 23F.
224. An immunogenic composition comprising a glycoconjugate from S. pneumoniae serotype 15B wherein said immunogenic composition does not comprise glycoconjugates from S. pneumoniae serotypes 1, 4, 5, 6A, 6B, 7F, 9V, 14, 15A, 15C, 18C, 19A, 19F and 23F.
225. An immunogenic composition comprising a glycoconjugate from S. pneumoniae serotype 15C wherein said immunogenic composition does not comprise glycoconjugates from S. pneumoniae serotypes 1, 4, 5, 6A, 6B, 7F, 9V, 14, 15A, 15B, 18C, 19A, 19F and 23F.
226. An immunogenic composition comprising a glycoconjugate from S. pneumoniae serotype 15A and a glycoconjugate from S. pneumoniae serotype 15C wherein said immunogenic composition does not comprise glycoconjugates from S. pneumoniae serotypes 1, 4, 5, 6A, 6B, 7F, 9V, 14, 15B, 18C, 19A, 19F and 23F.
227. An immunogenic composition comprising a glycoconjugate from S. pneumoniae serotype 15A and a glycoconjugate from S. pneumoniae serotype 15B wherein said immunogenic composition does not comprise glycoconjugates from S. pneumoniae serotypes 1, 4, 5, 6A, 6B, 7F, 9V, 14, 15C, 18C, 19A, 19F and 23F.
228. An immunogenic composition comprising a glycoconjugate from S. pneumoniae serotype 15B and a glycoconjugate from S. pneumoniae serotype 15C wherein said immunogenic composition does not comprise glycoconjugates from S. pneumoniae serotypes 1, 4, 5, 6A, 6B, 7F, 9V, 14, 15A, 18C, 19A, 19F and 23F.
229. An immunogenic composition comprising a glycoconjugate from S. pneumoniae serotype 15A wherein said immunogenic composition does not comprise glycoconjugates from S. pneumoniae serotypes 1, 3, 4, 5, 6A, 6B, 7F, 9V, 14, 15B, 15C, 18C, 19A, 19F and 23F.
230. An immunogenic composition comprising a glycoconjugate from S. pneumoniae serotype 15B wherein said immunogenic composition does not comprise glycoconjugates from S. pneumoniae serotypes 1, 3, 4, 5, 6A, 6B, 7F, 9V, 14, 15A, 15C, 18C, 19A, 19F and 23F.
231. An immunogenic composition comprising a glycoconjugate from S. pneumoniae serotype 15C wherein said immunogenic composition does not comprise glycoconjugates from S. pneumoniae serotypes 1, 3, 4, 5, 6A, 6B, 7F, 9V, 14, 15A, 15B, 18C, 19A, 19F and 23F.
232. An immunogenic composition comprising a glycoconjugate from S. pneumoniae serotype 15A and a glycoconjugate from S. pneumoniae serotype 15C wherein said immunogenic composition does not comprise glycoconjugates from S. pneumoniae serotypes 1, 3, 4, 5, 6A, 6B, 7F, 9V, 14, 15B, 18C, 19A, 19F and 23F.
233. An immunogenic composition comprising a glycoconjugate from S. pneumoniae serotype 15A and a glycoconjugate from S. pneumoniae serotype 15B wherein said immunogenic composition does not comprise glycoconjugates from S. pneumoniae serotypes 1, 3, 4, 5, 6A, 6B, 7F, 9V, 14, 15C, 18C, 19A, 19F and 23F.
234. An immunogenic composition comprising a glycoconjugate from S. pneumoniae serotype 15B and a glycoconjugate from S. pneumoniae serotype 15C wherein said immunogenic composition does not comprise glycoconjugates from S. pneumoniae serotypes 1, 3, 4, 5, 6A, 6B, 7F, 9V, 14, 15A, 18C, 19A, 19F and 23F.
235. An immunogenic composition comprising a glycoconjugate from S. pneumoniae serotype 15A wherein said immunogenic composition does not comprise glycoconjugates from S. pneumoniae serotypes 1, 4, 5, 6A, 6B, 7F, 9V, 14, 15B, 15C, 18C, 19A, 19F, 22F, 23F and 33F.
236. An immunogenic composition comprising a glycoconjugate from S. pneumoniae serotype 15B wherein said immunogenic composition does not comprise glycoconjugates from S. pneumoniae serotypes 1, 4, 5, 6A, 6B, 7F, 9V, 14, 15A, 15C, 18C, 19A, 19F, 22F, 23F and 33F.
237. An immunogenic composition comprising a glycoconjugate from S. pneumoniae serotype 15C wherein said immunogenic composition does not comprise glycoconjugates from S. pneumoniae serotypes 1, 4, 5, 6A, 6B, 7F, 9V, 14, 15A, 15B, 18C, 19A, 19F, 22F, 23F and 33F.
238. An immunogenic composition comprising a glycoconjugate from S. pneumoniae serotype 15A and a glycoconjugate from S. pneumoniae serotype 15C wherein said immunogenic composition does not comprise glycoconjugates from S. pneumoniae serotypes 1, 4, 5, 6A, 6B, 7F, 9V, 14, 15B, 18C, 19A, 19F, 22F, 23F and 33F.
239. An immunogenic composition comprising a glycoconjugate from S. pneumoniae serotype 15A and a glycoconjugate from S. pneumoniae serotype 15B wherein said immunogenic composition does not comprise glycoconjugates from S. pneumoniae serotypes 1, 4, 5, 6A, 6B, 7F, 9V, 14, 15C, 18C, 19A, 19F, 22F, 23F and 33F.
240. An immunogenic composition comprising a glycoconjugate from S. pneumoniae serotype 15B and a glycoconjugate from S. pneumoniae serotype 15C wherein said immunogenic composition does not comprise glycoconjugates from S. pneumoniae serotypes 1, 4, 5, 6A, 6B, 7F, 9V, 14, 15A, 18C, 19A, 19F, 22F, 23F and 33F.
241. An immunogenic composition comprising a glycoconjugate from S. pneumoniae serotype 15A wherein said immunogenic composition does not comprise glycoconjugates from S. pneumoniae serotypes 1, 3, 4, 5, 6A, 6B, 7F, 9V, 14, 15B, 15C, 18C, 19A, 19F, 22F, 23F and 33F.
242. An immunogenic composition comprising a glycoconjugate from S. pneumoniae serotype 15B wherein said immunogenic composition does not comprise glycoconjugates from S. pneumoniae serotypes 1, 3, 4, 5, 6A, 6B, 7F, 9V, 14, 15A, 15C, 18C, 19A, 19F, 22F, 23F and 33F.
243. An immunogenic composition comprising a glycoconjugate from S. pneumoniae serotype 15C wherein said immunogenic composition does not comprise glycoconjugates from S. pneumoniae serotypes 1, 3, 4, 5, 6A, 6B, 7F, 9V, 14, 15A, 15B, 18C, 19A, 19F, 22F, 23F and 33F.
244. An immunogenic composition comprising a glycoconjugate from S. pneumoniae serotype 15A and a glycoconjugate from S. pneumoniae serotype 15C wherein said immunogenic composition does not comprise glycoconjugates from S. pneumoniae serotypes 1, 3, 4, 5, 6A, 6B, 7F, 9V, 14, 15B, 18C, 19A, 19F, 22F, 23F and 33F.
245. An immunogenic composition comprising a glycoconjugate from S. pneumoniae serotype 15A and a glycoconjugate from S. pneumoniae serotype 15B wherein said immunogenic composition does not comprise glycoconjugates from S. pneumoniae serotypes 1, 3, 4, 5, 6A, 6B, 7F, 9V, 14, 15C, 18C, 19A, 19F, 22F, 23F and 33F.
246. An immunogenic composition comprising a glycoconjugate from S. pneumoniae serotype 15B and a glycoconjugate from S. pneumoniae serotype 15C wherein said immunogenic composition does not comprise glycoconjugates from S. pneumoniae serotypes 1, 3, 4, 5, 6A, 6B, 7F, 9V, 14, 15A, 18C, 19A, 19F, 22F, 23F and 33F.
247. An immunogenic composition comprising a glycoconjugate from S. pneumoniae serotype 15A wherein said immunogenic composition does not comprise glycoconjugates from S. pneumoniae serotypes 1, 4, 5, 6A, 6B, 7F, 8, 9V, 10A, 11A, 12F, 14, 15B, 15C, 18C, 19A, 19F, 22F, 23F and 33F.
248. An immunogenic composition comprising a glycoconjugate from S. pneumoniae serotype 15B wherein said immunogenic composition does not comprise glycoconjugates from S. pneumoniae serotypes 1, 4, 5, 6A, 6B, 7F, 8, 9V, 10A, 11A, 12F, 14, 15A, 15C, 18C, 19A, 19F, 22F, 23F and 33F.
249. An immunogenic composition comprising a glycoconjugate from S. pneumoniae serotype 15C wherein said immunogenic composition does not comprise glycoconjugates from S. pneumoniae serotypes 1, 4, 5, 6A, 6B, 7F, 8, 9V, 10A, 11A, 12F, 14, 15A, 15B, 18C, 19A, 19F, 22F, 23F and 33F.
250. An immunogenic composition comprising a glycoconjugate from S. pneumoniae serotype 15A and a glycoconjugate from S. pneumoniae serotype 15C wherein said immunogenic composition does not comprise glycoconjugates from S. pneumoniae serotypes 1, 4, 5, 6A, 6B, 7F, 8, 9V, 10A, 11A, 12F, 14, 15B, 18C, 19A, 19F, 22F, 23F and 33F
251. An immunogenic composition comprising a glycoconjugate from S. pneumoniae serotype 15A and a glycoconjugate from S. pneumoniae serotype 15B wherein said immunogenic composition does not comprise glycoconjugates from S. pneumoniae serotypes 1, 4, 5, 6A, 6B, 7F, 8, 9V, 10A, 11A, 12F, 14, 15C, 18C, 19A, 19F, 22F, 23F and 33F.
252. An immunogenic composition comprising a glycoconjugate from S. pneumoniae serotype 15B and a glycoconjugate from S. pneumoniae serotype 15C wherein said immunogenic composition does not comprise glycoconjugates from S. pneumoniae serotypes 1, 4, 5, 6A, 6B, 7F, 8, 9V, 10A, 11A, 12F, 14, 15A, 18C, 19A, 19F, 22F, 23F and 33F.
253. An immunogenic composition comprising a glycoconjugate from S. pneumoniae serotype 15A wherein said immunogenic composition does not comprise glycoconjugates from S. pneumoniae serotypes 1, 4, 5, 6A, 6B, 7F, 8, 9V, 10A, 11A, 12F, 14, 15B, 18C, 19A, 19F, 22F, 23F and 33F.
254. An immunogenic composition comprising a glycoconjugate from S. pneumoniae serotype 15C wherein said immunogenic composition does not comprise glycoconjugates from S. pneumoniae serotypes 1, 4, 5, 6A, 6B, 7F, 8, 9V, 10A, 11A, 12F, 14, 15B, 18C, 19A, 19F, 22F, 23F and 33F.
255. An immunogenic composition comprising a glycoconjugate from S. pneumoniae serotype 15A wherein said immunogenic composition does not comprise glycoconjugates from S. pneumoniae serotypes 1, 3, 4, 5, 6A, 6B, 7F, 8, 9V, 10A, 11A, 12F, 14, 15B, 15C, 18C, 19A, 19F, 22F, 23F and 33F.
256. An immunogenic composition comprising a glycoconjugate from S. pneumoniae serotype 15B wherein said immunogenic composition does not comprise glycoconjugates from S. pneumoniae serotypes 1, 3, 4, 5, 6A, 6B, 7F, 8, 9V, 10A, 11A, 12F, 14, 15A, 15C, 18C, 19A, 19F, 22F, 23F and 33F.
257. An immunogenic composition comprising a glycoconjugate from S. pneumoniae serotype 15C wherein said immunogenic composition does not comprise glycoconjugates from S. pneumoniae serotypes 1, 3, 4, 5, 6A, 6B, 7F, 8, 9V, 10A, 11A, 12F, 14, 15A, 15B, 18C, 19A, 19F, 22F, 23F and 33F.
258. An immunogenic composition comprising a glycoconjugate from S. pneumoniae serotype 15A and a glycoconjugate from S. pneumoniae serotype 15C wherein said immunogenic composition does not comprise glycoconjugates from S. pneumoniae serotypes 1, 3, 4, 5, 6A, 6B, 7F, 8, 9V, 10A, 11A, 12F, 14, 15B, 18C, 19A, 19F, 22F, 23F and 33F
259. An immunogenic composition comprising a glycoconjugate from S. pneumoniae serotype 15A and a glycoconjugate from S. pneumoniae serotype 15B wherein said immunogenic composition does not comprise glycoconjugates from S. pneumoniae serotypes 1, 3, 4, 5, 6A, 6B, 7F, 8, 9V, 10A, 11A, 12F, 14, 15C, 18C, 19A, 19F, 22F, 23F and 33F.
260. An immunogenic composition comprising a glycoconjugate from S. pneumoniae serotype 15B and a glycoconjugate from S. pneumoniae serotype 15C wherein said immunogenic composition does not comprise glycoconjugates from S. pneumoniae serotypes 1, 3, 4, 5, 6A, 6B, 7F, 8, 9V, 10A, 11A, 12F, 14, 15A, 18C, 19A, 19F, 22F, 23F and 33F.
261. An immunogenic composition comprising a glycoconjugate from S. pneumoniae serotype 15A wherein said immunogenic composition does not comprise glycoconjugates from S. pneumoniae serotypes 1, 3, 4, 5, 6A, 6B, 7F, 8, 9V, 10A, 11A, 12F, 14, 15B, 18C, 19A, 19F, 22F, 23F and 33F.
262. An immunogenic composition comprising a glycoconjugate from S. pneumoniae serotype 15C wherein said immunogenic composition does not comprise glycoconjugates from S. pneumoniae serotypes 1, 3, 4, 5, 6A, 6B, 7F, 8, 9V, 10A, 11A, 12F, 14, 15B, 18C, 19A, 19F, 22F, 23F and 33F.
263. The immunogenic composition of any one of paragraphs 205-262 wherein the glycoconjugates are individually conjugated to the carrier protein.
264. The immunogenic composition of any one of paragraphs 205-263 wherein if present, the glycoconjugate from S. pneumoniae serotype 15C is conjugated to CRM₁₉₇.
265. The immunogenic composition of any one of paragraphs 205-264 wherein if present, the glycoconjugate from S. pneumoniae serotype 15A is conjugated to CRM₁₉₇.
266. The immunogenic composition of any one of paragraphs 205-265 wherein if present, the glycoconjugate from S. pneumoniae serotype 15B is conjugated to CRM₁₉₇.
267. The immunogenic composition of any one of paragraphs 205-266 comprising glycoconjugates from 2 to 25 different serotypes of S. pneumoniae.
268. The immunogenic composition of any one of paragraphs 205-267 comprising glycoconjugates from 2 different pneumococcal serotypes.
269. The immunogenic composition of any one of paragraphs 205-267 comprising glycoconjugates from 3 different pneumococcal serotypes.
270. The immunogenic composition of any one of paragraphs 205-267 comprising glycoconjugates from 7 different pneumococcal serotypes.
271. The immunogenic composition of any one of paragraphs 205-267 comprising glycoconjugates from 8 different pneumococcal serotypes.
272. The immunogenic composition of any one of paragraphs 205-267 comprising glycoconjugates from 9 different pneumococcal serotypes.
273. The immunogenic composition of any one of paragraphs 205-267 comprising glycoconjugates from 10 different pneumococcal serotypes.
274. The immunogenic composition of any one of paragraphs 205-267 comprising glycoconjugates from 11 different pneumococcal serotypes.
275. The immunogenic composition of any one of paragraphs 205-267 comprising glycoconjugates from 12 different pneumococcal serotypes.
276. The immunogenic composition of any one of paragraphs 205-267 comprising glycoconjugates from 13 different pneumococcal serotypes.
277. The immunogenic composition of any one of paragraphs 205-267 comprising glycoconjugates from 14 different pneumococcal serotypes.
278. The immunogenic composition of any one of paragraphs 205-267 comprising glycoconjugates from 15 different pneumococcal serotypes.
279. The immunogenic composition of any one of paragraphs 205-267 comprising glycoconjugates from 16 different pneumococcal serotypes.
280. The immunogenic composition of any one of paragraphs 205-267 comprising glycoconjugates from 17 different pneumococcal serotypes.
281. The immunogenic composition of any one of paragraphs 205-267 comprising glycoconjugates from 18 different pneumococcal serotypes.
282. The immunogenic composition of any one of paragraphs 205-267 comprising glycoconjugates from 19 different pneumococcal serotypes.
283. The immunogenic composition of any one of paragraphs 205-267 comprising glycoconjugates from 19 different pneumococcal serotypes.
284. The immunogenic composition of any one of paragraphs 205-267 comprising glycoconjugates from 19 different pneumococcal serotypes.
285. The immunogenic composition of any one of paragraphs 205-267 comprising glycoconjugates from 20 different pneumococcal serotypes.
286. The immunogenic composition of any one of paragraphs 205-267 comprising glycoconjugates from 21 different pneumococcal serotypes.
287. The immunogenic composition of any one of paragraphs 205-267 comprising glycoconjugates from 22 different pneumococcal serotypes.
288. The immunogenic composition of any one of paragraphs 205-267 comprising glycoconjugates from 23 different pneumococcal serotypes.
289. The immunogenic composition of any one of paragraphs 205-267 comprising glycoconjugates from 24 different pneumococcal serotypes.
290. The immunogenic composition of any one of paragraphs 205-267 comprising glycoconjugates from 25 different pneumococcal serotypes.
291. The immunogenic composition of any one of paragraphs 205-267 which is a 2, 3, 4, 5, 6, 7, 8, 9, 10, 11, 12, 13, 14, 15, 16, 17, 18, 19 or 20-valent pneumococcal conjugate composition.
292. The immunogenic composition of any one of paragraphs 205-267 which is a 2-valent pneumococcal conjugate composition.
293. The immunogenic composition of any one of paragraphs 205-267 which is a 3-valent pneumococcal conjugate composition.
294. The immunogenic composition of any one of paragraphs 205-267 which is a 4-valent pneumococcal conjugate composition.
295. The immunogenic composition of any one of paragraphs 205-267 which is a 5-valent pneumococcal conjugate composition.
296. The immunogenic composition of any one of paragraphs 205-267 which is a 6-valent pneumococcal conjugate composition.
297. The immunogenic composition of any one of paragraphs 205-267 which is a 7-valent pneumococcal conjugate composition.
298. The immunogenic composition of any one of paragraphs 205-267 which is an 8-valent pneumococcal conjugate composition.
299. The immunogenic composition of any one of paragraphs 205-267 which is a 9-valent pneumococcal conjugate composition.
300. The immunogenic composition of any one of paragraphs 205-267 which is a 10-valent pneumococcal conjugate composition.
301. The immunogenic composition of any one of paragraphs 205-267 which is a 11-valent pneumococcal conjugate composition.
302. The immunogenic composition of any one of paragraphs 205-267 which is a 12-valent pneumococcal conjugate composition.
303. The immunogenic composition of any one of paragraphs 205-267 which is a 13-valent pneumococcal conjugate composition.
304. The immunogenic composition of any one of paragraphs 205-267 which is a 14-valent pneumococcal conjugate composition.
305. The immunogenic composition of any one of paragraphs 205-267 which is a 15-valent pneumococcal conjugate composition.
306. The immunogenic composition of any one of paragraphs 205-267 which is a 16-valent pneumococcal conjugate composition.
307. The immunogenic composition of any one of paragraphs 205-267 which is a 17-valent pneumococcal conjugate composition.
308. The immunogenic composition of any one of paragraphs 205-267 which is an 18-valent pneumococcal conjugate composition.
309. The immunogenic composition of any one of paragraphs 205-267 which is a 19-valent pneumococcal conjugate composition.
310. The immunogenic composition of any one of paragraphs 205-267 which is a 20-valent pneumococcal conjugate composition.
311. The immunogenic composition of any one of paragraphs 205-267 which is a 21-valent pneumococcal conjugate composition.
312. The immunogenic composition of any one of paragraphs 205-267 which is a 22-valent pneumococcal conjugate composition.
313. The immunogenic composition of any one of paragraphs 205-267 which is a 23-valent pneumococcal conjugate composition.
314. The immunogenic composition of any one of paragraphs 205-267 which is a 24-valent pneumococcal conjugate composition.
315. The immunogenic composition of any one of paragraphs 205-314 wherein all the glycoconjugates are individually conjugated to the carrier protein.
316. The immunogenic composition of any one of paragraphs 205-314 wherein the glycoconjugates are individually conjugated to CRM₁₉₇.
317. The immunogenic composition of any one of paragraphs 205-314 wherein the glycoconjugates are all individually conjugated to CRM₁₉₇.
318. The immunogenic composition of any one of paragraphs 205-317 for use in a method of complementing a vaccine which comprises glycoconjugates from S. pneumoniae serotypes 1, 4, 5, 6B, 7F, 9V, 14, 18C, 19F and 23F.
319. The immunogenic composition of any one of paragraphs 205-317 for use in a method of complementing a 10-valent pneumococcal conjugate vaccine wherein said conjugates consist of glycoconjugates from S. pneumoniae serotypes 1, 4, 5, 6B, 7F, 9V, 14, 18C, 19F and 23F.
320. The immunogenic composition for use of paragraph 305 wherein said glycoconjugates from S. pneumoniae serotypes 1, 4, 5, 6B, 7F, 9V, 14 and 23F are conjugated to PD, said glycoconjugate from S. pneumoniae serotype 18C is conjugated to TT and said glycoconjugate from S. pneumoniae serotype 19F is conjugated to DT.
321. The immunogenic composition of any one of paragraphs 205-317 for use in a method of complementing a vaccine which comprises glycoconjugates from S. pneumoniae serotypes 1, 4, 5, 6A, 6B, 7F, 9V, 14, 18C, 19A, 19F and 23F.
322. The immunogenic composition of any one of paragraphs 205-317 for use in a method of complementing a 12-valent pneumococcal conjugate vaccine wherein said conjugates consist of glycoconjugates from S. pneumoniae serotypes 1, 4, 5, 6A, 6B, 7F, 9V, 14, 18C, 19A, 19F and 23F conjugated to CRM₁₉₇.
323. The immunogenic composition of any one of paragraphs 205-317 for use in a method of complementing a vaccine which comprises glycoconjugates from S. pneumoniae serotypes 1, 3, 4, 5, 6A, 6B, 7F, 9V, 14, 18C, 19A, 19F and 23F.
324. The immunogenic composition of any one of paragraphs 205-317 for use in a method of complementing a 13-valent pneumococcal conjugate vaccine wherein said conjugates consist of glycoconjugates from S. pneumoniae serotypes 1, 3, 4, 5, 6A, 6B, 7F, 9V, 14, 18C, 19A, 19F and 23F conjugated to CRM₁₉₇.
325. The immunogenic composition of any one of paragraphs 205-317 for use in a method of complementing a vaccine which comprises glycoconjugates from S. pneumoniae serotypes 1, 4, 5, 6A, 6B, 7F, 9V, 14, 18C, 19A, 19F, 22F, 23F and 33F.
326. The immunogenic composition of any one of paragraphs 205-317 for use in a method of complementing a 14-valent pneumococcal conjugate vaccine wherein said conjugates consist of glycoconjugates from S. pneumoniae serotypes 1, 4, 5, 6A, 6B, 7F, 9V, 14, 18C, 19A, 19F, 22F, 23F and 33F conjugated to CRM₁₉₇.
327. The immunogenic composition of any one of paragraphs 205-317 for use in a method of complementing a vaccine which comprises glycoconjugates from S. pneumoniae serotypes 1, 3, 4, 5, 6A, 6B, 7F, 9V, 14, 18C, 19A, 19F, 22F, 23F and 33F.
328. The immunogenic composition of any one of paragraphs 205-317 for use in a method of complementing a 15-valent pneumococcal conjugate vaccine wherein said conjugates consist of glycoconjugates from S. pneumoniae serotypes 1, 3, 4, 5, 6A, 6B, 7F, 9V, 14, 18C, 19A, 19F, 22F, 23F and 33F conjugated to CRM₁₉₇.
329. The immunogenic composition of any one of paragraphs 205-317 for use in a method of complementing a vaccine which comprises glycoconjugates from S. pneumoniae serotypes 1, 4, 5, 6A, 6B, 7F, 8, 9V, 10A, 11A, 12F, 14, 15B, 18C, 19A, 19F, 22F, 23F and 33F.
330. The immunogenic composition of any one of paragraphs 205-317 for use in a method of complementing a 19-valent pneumococcal conjugate vaccine wherein said conjugates consist of glycoconjugates from S. pneumoniae serotypes 1, 4, 5, 6A, 6B, 7F, 8, 9V, 10A, 11A, 12F, 14, 15B, 18C, 19A, 19F, 22F, 23F and 33F conjugated to CRM₁₉₇.
331. The immunogenic composition of any one of paragraphs 205-317 for use in a method of complementing a vaccine which comprises glycoconjugates from S. pneumoniae serotypes 1, 3, 4, 5, 6A, 6B, 7F, 8, 9V, 10A, 11A, 12F, 14, 15B, 18C, 19A, 19F, 22F, 23F and 33F.
332. The immunogenic composition of any one of paragraphs 205-317 for use in a method of complementing a 20-valent pneumococcal conjugate vaccine wherein said conjugates consist of glycoconjugates from S. pneumoniae serotypes 1, 3, 4, 5, 6A, 6B, 7F, 8, 9V, 10A, 11A, 12F, 14, 15B, 18C, 19A, 19F, 22F, 23F and 33F.
333. The immunogenic composition for use of paragraphs 318 wherein said glycoconjugates from S. pneumoniae serotypes 1, 3, 4, 5, 6A, 6B, 7F, 8, 9V, 10A, 11A, 12F, 14, 15B, 18C, 19A, 19F, 22F, 23F and 33F are conjugated to CRM₁₉₇.
334. The immunogenic composition of any one of paragraphs 210, 216, 222, 228, 234, 240, 246, 252 or 260 for use in a method of complementing a pneumococcal conjugate vaccine which comprises a glycoconjugate from S. pneumoniae serotype 15A but which does not comprise glycoconjugates from S. pneumoniae serotypes 15B and 15C.
335. The immunogenic composition of any one of paragraphs 208, 214, 220, 226, 232, 238, 244, 250 or 258 for use in a method of complementing a pneumococcal conjugate vaccine which comprises a glycoconjugate from S. pneumoniae serotype 15B but which does not comprise glycoconjugates from S. pneumoniae serotypes 15A and 15C.
336. The immunogenic composition of any one of paragraphs 209, 215, 221, 227, 233, 239, 245, 251 or 259 for use in a method of complementing a pneumococcal conjugate vaccine which comprises a glycoconjugate from S. pneumoniae serotype 15C but which does not comprise glycoconjugates from S. pneumoniae serotypes 15A and 15B.
337. The immunogenic composition of any one of paragraphs 207, 213, 219, 225, 231, 237, 243, 249, 254, 257 or 262 for use in a method of complementing a pneumococcal conjugate vaccine which comprises a glycoconjugate from S. pneumoniae serotype 15A and a glycoconjugate from S. pneumoniae serotype 15B but which does not comprise a glycoconjugate from S. pneumoniae serotypes 15C.
338. The immunogenic composition of any one of paragraphs 207, 213, 219, 225, 231, 237, 243, 249, 254, 257 or 262 for use in a method of complementing a pneumococcal conjugate vaccine which comprises a glycoconjugate from S. pneumoniae serotype 15A but which does not comprise a glycoconjugate from S. pneumoniae serotypes 15C.
339. The immunogenic composition of any one of paragraphs 207, 213, 219, 225, 231, 237, 243, 249, 254, 257 or 262 for use in a method of complementing a pneumococcal conjugate vaccine which comprises a glycoconjugate from S. pneumoniae serotype 15B but which does not comprise a glycoconjugate from S. pneumoniae serotypes 15C.
340. The immunogenic composition of any one of paragraphs 206, 212, 218, 224, 230, 236, 242, 248 or 256 for use in a method of complementing a pneumococcal conjugate vaccine which comprises a glycoconjugate from S. pneumoniae serotype 15A and a glycoconjugate from S. pneumoniae serotype 15C but which does not comprise a glycoconjugate from S. pneumoniae serotypes 15B.
341. The immunogenic composition of any one of paragraphs 206, 212, 218, 224, 230, 236, 242, 248 or 256 for use in a method of complementing a pneumococcal conjugate vaccine which comprises a glycoconjugate from S. pneumoniae serotype 15A but which does not comprise a glycoconjugate from S. pneumoniae serotypes 15B.
342. The immunogenic composition of any one of paragraphs 206, 212, 218, 224, 230, 236, 242, 248 or 256 for use in a method of complementing a pneumococcal conjugate vaccine which comprises a glycoconjugate from S. pneumoniae serotype 15C but which does not comprise a glycoconjugate from S. pneumoniae serotypes 15B.
343. The immunogenic composition of any one of paragraphs 205, 211, 217, 223, 229, 235, 241, 247, 253, 255 or 261 for use in a method of complementing a pneumococcal conjugate vaccine which comprises a glycoconjugate from S. pneumoniae serotype 15B and a glycoconjugate from S. pneumoniae serotype 15C but which does not comprise a glycoconjugate from S. pneumoniae serotypes 15A.
344. The immunogenic composition of any one of paragraphs 205, 211, 217, 223, 229, 235, 241, 247, 253, 255 or 261 for use in a method of complementing a pneumococcal conjugate vaccine which comprises a glycoconjugate from S. pneumoniae serotype 15B but which does not comprise a glycoconjugate from S. pneumoniae serotypes 15A.
345. The immunogenic composition of any one of paragraphs 205, 211, 217, 223, 229, 235, 241, 247, 253, 255 or 261 for use in a method of complementing a pneumococcal conjugate vaccine which comprises a glycoconjugate from S. pneumoniae serotype 15C but which does not comprise a glycoconjugate from S. pneumoniae serotypes 15A.
346. A kit comprising an immunogenic composition according to any one of paragraphs 1-65 or a set of immunogenic compositions according to any one of paragraphs 66-204 and an information leaflet.
347. The kit of paragraph 346 wherein said information leaflet mentions the ability of the composition or set of immunogenic compositions to elicit functional antibodies against S. pneumoniae serotypes 15A, 15B and 15C.
348. The kit of paragraph 346 or 347 wherein said information leaflet mentions the ability of the composition or set of immunogenic compositions to elicit anti-capsular antibodies against S. pneumoniae serotypes 15A, 15B and 15C at a concentration≥0.35 μg/mL in a human population.
349. The kit of any one of paragraphs 346-348 wherein said information leaflet mentions the ability of the composition or set of immunogenic compositions to elicit OPA titers against S. pneumoniae serotypes 15A, 15B and 15C in a human population.
350. A process for producing a kit comprising an immunogenic composition and an information leaflet, said process comprising the step of:
   producing an immunogenic composition according to any one of paragraphs 1-65 and
   combining in the same kit said immunogenic composition and information leaflet, wherein said information leaflet mentions the ability of said composition to elicit functional antibodies against S. pneumoniae serotypes 15A, 15B and 15C.
351. A process for producing a kit comprising a set of immunogenic compositions according to any one of paragraphs 66-204 and an information leaflet, said process comprising the step of:
   producing a set of immunogenic compositions and
   combining in the same kit said set of immunogenic compositions and information leaflet, wherein said information leaflet mentions the ability of said set of immunogenic compositions to elicit functional antibodies against S. pneumoniae serotypes 15A, 15B and 15C.
352. A process for producing a kit comprising an immunogenic composition according to any one of paragraphs 1-65 and an information leaflet, said process comprising the step of:
   producing an immunogenic composition according to any one of paragraphs 1-65 and
   combining in the same kit said immunogenic composition and information leaflet, wherein said information leaflet mentions the ability of the composition to elicit anti-capsular antibodies against S. pneumoniae serotypes 15A, 15B and 15C at a concentration 0.35 μg/mL in a human population.
353. A process for producing a kit comprising a set of immunogenic compositions according to any one of paragraphs 66-204 and an information leaflet, said process comprising the step of:
   producing a set of immunogenic compositions according to any one of paragraphs 66-204 and
   combining in the same kit said set of immunogenic compositions and information leaflet, wherein said information leaflet mentions the ability of the set of immunogenic compositions to elicit anti-capsular antibodies against S. pneumoniae serotypes 15A, 15B and 15C at a concentration 0.35 μg/mL in a human population.
354. A process for producing a kit comprising an immunogenic composition according to any one of paragraphs 1-65 and an information leaflet, said process comprising the step of:
   producing an immunogenic composition according to any one of paragraphs 1-65 and
   combining in the same kit said immunogenic composition and information leaflet, wherein said information leaflet mentions the ability of the composition to elicit OPA titers against S. pneumoniae serotypes 15A, 15B and 15C in a human population.
355. A process for producing a kit comprising a set of immunogenic compositions according to any one of paragraphs 66-204 and an information leaflet, said process comprising the step of:
   producing a set of immunogenic compositions according to any one of paragraphs 66-204 and
   combining in the same kit said set of immunogenic compositions and information leaflet, wherein said information leaflet mentions the ability of the set of immunogenic compositions to elicit OPA titers against S. pneumoniae serotypes 15A, 15B and 15C in a human population.
356. A process for producing a kit comprising an immunogenic composition and an information leaflet, said process comprising the step of:
   producing an immunogenic composition according to any one of paragraphs 1-65;
   printing an information leaflet wherein said information leaflet mentions the ability of said composition to elicit functional antibodies against S. pneumoniae serotypes 15A, 15B and 15C;
   combining in the same kit said immunogenic composition and said information leaflet.
357. A process for producing a kit comprising a set of immunogenic compositions and an information leaflet, said process comprising the step of:
   producing a set of immunogenic compositions according to any one of paragraphs 66-204;
   printing an information leaflet wherein said information leaflet mentions the ability of said set of immunogenic compositions to elicit functional antibodies against S. pneumoniae serotypes 15A, 15B and 15C;
   combining in the same kit said set of immunogenic compositions and said information leaflet.
358. A process for producing a kit comprising an immunogenic composition and an information leaflet, said process comprising the step of:
   producing an immunogenic composition according to any one of paragraphs 1-65;
   printing an information leaflet wherein said information leaflet mentions the ability of the composition to elicit anti-capsular antibodies against S. pneumoniae serotypes 15A, 15B and 15C at a concentration 0.35 μg/mL in a human population;
   combining in the same kit said immunogenic composition and said information leaflet.
359. A process for producing a kit comprising a set of immunogenic compositions and an information leaflet, said process comprising the step of:
   producing a set of immunogenic compositions according to any one of paragraphs 66-204;
   printing an information leaflet wherein said information leaflet mentions the ability of the set of immunogenic compositions to elicit anti-capsular antibodies against S. pneumoniae serotypes 15A, 15B and 15C at a concentration≥0.35 μg/mL in a human population;
   combining in the same kit said set of immunogenic compositions and said information leaflet.
360. A process for producing a kit comprising an immunogenic composition and an information leaflet, said process comprising the step of:
   producing an immunogenic composition according to any one of paragraphs 1-65;
   printing an information leaflet wherein said information leaflet mentions the ability of the composition to elicit OPA titers against S. pneumoniae serotypes 15A, 15B and 15C in a human population;
   combining in the same kit said immunogenic composition and said information leaflet.
361. A process for producing a kit comprising a set of immunogenic compositions and an information leaflet, said process comprising the step of:
   producing a set of immunogenic compositions according to any one of paragraphs 1-65;
   printing an information leaflet wherein said information leaflet mentions the ability of the set of immunogenic compositions to elicit OPA titers against S. pneumoniae serotypes 15A, 15B and 15C in a human population;
   combining in the same kit said set of immunogenic compositions and said information leaflet.

As used herein, the term “about” means within a statistically meaningful range of a value, such as a stated concentration range, time frame, molecular weight, temperature or pH. Such a range can be within an order of magnitude, typically within 20%, more typically within 10%, and even more typically within 5% or within 1% of a given value or range. Sometimes, such a range can be within the experimental error typical of standard methods used for the measurement and/or determination of a given value or range. The allowable variation encompassed by the term “about” will depend upon the particular system under study, and can be readily appreciated by one of ordinary skill in the art. Whenever a range is recited within this application, every whole number integer within the range is also contemplated as an embodiment of the disclosure.

The terms “comprising”, “comprise” and “comprises” herein are intended by the inventors to be optionally substitutable with the terms “consisting of”, “consist of” and “consists of”, respectively, in every instance.

All references or patent applications cited within this patent specification are incorporated by reference herein.

The invention is illustrated in the accompanying examples. The examples below are carried out using standard techniques, which are well known and routine to those of skill in the art, except where otherwise described in detail. The examples are illustrative, but do not limit the invention.

### EXAMPLE

### Example 1. Evaluation of Cross-Reactive Opsonophagocytic Immune Responses within Serogroup 15 of Streptococcus pneumoniae

Materials and Methods

Sera

Sera from adults vaccinated with a 20-valent pneumococcal conjugate vaccine (20vPnC) were tested in OPA assays for the serotypes 15B in 15C and 15A.

The sera were collected from U.S. clinical trials B7471001 (N=33, 1 month post-vaccinated) and B7471002 (N=84, matched pre- and post-vaccination), respectively.

Study B7471001 (ClinicalTrials.gov Identifier: NCT02955160) was a phase 1, first-in-human, randomized, controlled, observer-blinded study with a 2-arm parallel design. Healthy adults aged 18 to 49 years of age received either a single intramuscular dose of a 20-valent pneumococcal conjugate vaccine or a licensed tetanus, diphtheria, acellular pertussis combination vaccine (Tdap) (control group). (see https://clinicaltrials.gov/ct2/show/NCT02955160, accessed on Sep. 22, 2020).

Study B7471002 (ClinicalTrials.gov Identifier: NCT03313037) was a phase 2, randomized, double-blinded study with a 2-arm parallel design. Healthy adults aged 60 through 64 years of age received either a single intramuscular dose of a 20-valent pneumococcal conjugate vaccine followed 1 month later with a dose of saline or Prevnar 13 (13vPnC) followed 1 month later with a dose of PPSV23 (control group). (see https://clinicaltrials.gov/ct2/show/NCT03313037, accessed on Sep. 22, 2020).

The 20-valent pneumococcal conjugate vaccine (20vPnC) tested in these studies contained conjugates from pneumococcal serotypes 1, 3, 4, 5, 6A, 6B, 7F, 8, 9V, 10A, 11A, 12F, 14, 15B, 18C, 19A, 19F, 22F, 23F and 33F, individually conjugated to diphtheria cross-reacting material 197 (CRM₁₉₇) carrier protein.

The 13-valent pneumococcal conjugate vaccine (13vPnC) tested contained conjugates from pneumococcal serotypes 1, 3, 4, 5, 6A, 6B, 7F, 9V, 14, 18C, 19A, 19F, and 23F, individually conjugated to diphtheria cross-reacting material 197 (CRM₁₉₇) carrier protein (Prevenar 13®).

Microcolony Opsonophagocytic Assay (mcOPA) Procedure

Opsonophagocytic assays (OPAs) are used to measure functional antibodies in human sera against S. pneumoniae serotypes 15A, 15B and 15C. Test serum is set up in assay reactions that measure the ability of capsular polysaccharide specific immunoglobulin to opsonize bacteria, trigger complement deposition, thereby facilitating phagocytosis and killing of bacteria by phagocytes. The OPA titer is defined as the reciprocal dilution that results in a 50% reduction in bacterial count over control wells without test serum. The OPA titer is interpolated from the two dilutions that encompass this 50% killing cut-off. OPA procedures were based on methods described in Hu et al., (2005) Clin Diagn Lab Immunol 12:287-295.

Microcolony Opsonophagocytic Assay quantitatively assesses functional anti-S. pneumoniae antibodies by measuring bacterial survival in microcolony OPA (mcOPA) reactions containing the test serum. Serial dilutions (2.5-fold) of heat-inactivated serum were added to the target bacteria in assay plates and incubated for 30 minutes with shaking. Baby rabbit complement (3-4 week old, 12.5% final concentration) and differentiated HL-60 cells, were then added to each well at an approximate effector to target ratio of 200:1 and incubated at 37° C. with shaking. To terminate the reaction, 80 μL of 0.9% NaCl was added to all wells, the reaction solution was mixed, and a 10-μL aliquot were transferred to the wells of Millipore, MultiScreenHTS HV filter plates containing 200 μL of water. Liquid was filtered through the plates under vacuum, and 150 μL of HySoy medium was added to each well and filtered through.

Following filtration, the filter plates were incubated overnight in a 5% C02 incubator at 37° C. and were then fixed with Destain Solution (Bio-Rad). The plates were then stained with Coomassie Blue and destained once. Colonies were imaged and enumerated on a Cellular Technology Limited (CTL) ImmunoSpot Analyzer®. The OPA antibody titer was determined as the reciprocal of the lowest serum dilution resulting in 50% reduction in the number of bacterial colonies when compared to the bacteria-effector cell-complement control wells that did not contain serum.

### Example 2. Streptococcus pneumoniae Serotype 15B, 15C, 15A Cross-Functional OPAs

Cross-reactive antibodies that have functional, eg opsonophagocytic, activity are termed cross-functional. Cross-functional responses between Streptococcus pneumoniae serotypes 15A, 15B and 15C were evaluated. Opsonophagocytic assays were developed against serotype 15A, serotype 15B and serotype 15C strains. Evaluation of sera from clinical trials B7471001 and B7471002 (see Example 1) demonstrated that immunization with a vaccine containing a 15B capsular polysaccharide conjugate elicited strong cross-functional responses against serotype 15C isolates, but only minor cross-functional responses against serotype 15A.

Results—OPA Responses in 15B, 15C and 15A

The cross-functional response of immune sera from adults immunized with either 20vPnC or Tdap against serotypes 15B, 15C and 15A, was evaluated in the respective microcolony Opsonophagocytic Assays (mcOPAs), along with the homologous functional response to serotype 15B.

Serum panel from adults immunized with 20vPnC or Tdap (study B7471001 (see above)) was evaluated on OPA for the homologous response to serotype 15B and for cross-reactivity of anti-15B antibodies to serotypes 15C and 15A.

As shown in Table 1, serum from subjects immunized with 20vPnC were able to elicit killing of both 15C strains. Serum from subjects immunized with 20vPnC were able to elicit some killing of both 15A strains, however the titers were low.

TABLE 1
B7471001 FIH Study 15B, 15C and
15A Cross functional Responses

| Serotype | Vaccine Group | # of samples | GMT |
|---|---|---|---|
| 15B | Tdap | 33 | 34 |
| 15B | 20vPnC | 32 | 19334 |
| 15C (Strain 1) | Tdap | 33 | 9 |
| 15C (Strain 1) | 20vPnC | 32 | 1692 |
| 15C (Strain 2) | Tdap | 33 | 12 |
| 15C (Strain 2) | 20vPnC | 32 | 2747 |
| 15A (Strain 1) | Tdap | 33 | 6 |
| 15A (Strain 1) | 20vPnC | 32 | 10 |
| 15A (Strain 2) | Tdap | 33 | 6 |
| 15A (Strain 2) | 20vPnC | 32 | 30 |

### Example 3. Streptococcus pneumoniae Serotype 15B and 15C Cross-Functional Response

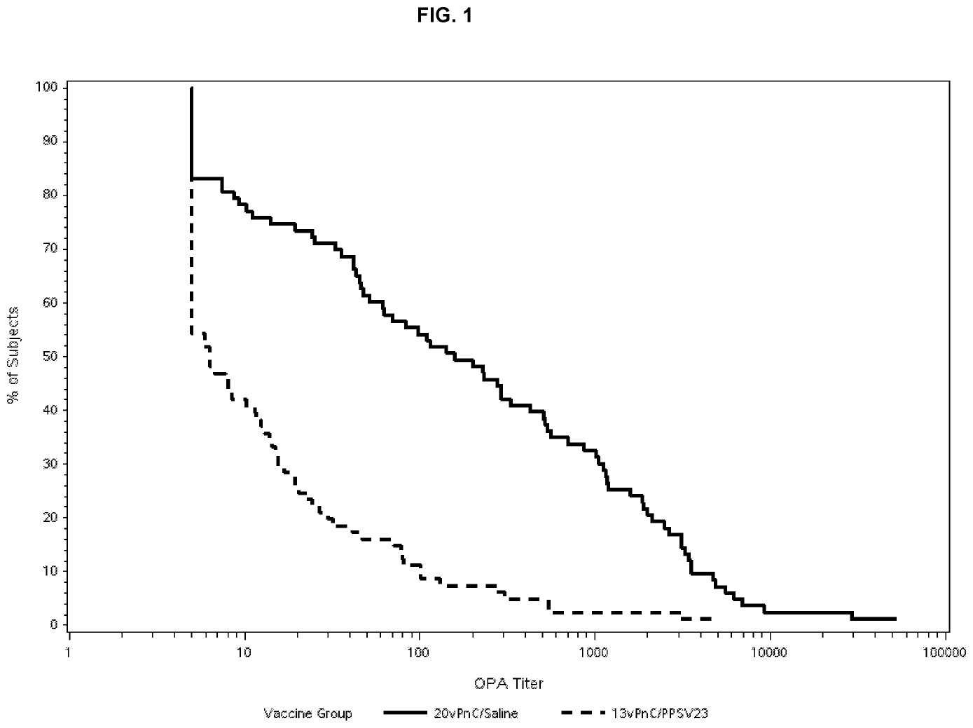

To confirm the results seen at Example 2 with a larger sample set, cross-functional 15C OPAs were performed using sera from vaccinees administered 20vPnC, which contains 15B capsular polysaccharide conjugate, or 13vPnC, which does not contain 15B capsular polysaccharide conjugate. GMTs and 95% correlation coefficients were determined for the two arms of the study. GMR (Geometric Mean Ratio) was also calculated to compare the two groups. The 15C OPA GMT for the 20vPnC group was 164, and for the 13vPnC group was 14 (Table 2). The GMR between the groups was 11.9 (Table 2), indicating that the 15B-containing 20vPnC elicited cross-functional immune responses to 15C. There is also a separation in the 15C OPA titer Reverse Cumulative Distribution Curve (RCDC) for the 20vPnC and 13vPnC groups (FIG. 1).

TABLE 2
B7471002: Pn15C-mcOPA Response

| Vaccine Group | N^{a} | GMT^{b} | (95% CI)^{c} |
|---|---|---|---|
| 20vPnC | 83 | 164 | (94.2, 286.8) |
| 13vPnC | 81 | 14 | (9.9, 19.2) |

| | GMR^{d} | (95% CI)^{e} |
|---|---|---|
| Vaccine Comparison | 11.9 | (6.25, 22.84) |

Abbreviations: GMR = geometric mean ratio; GMT = geometric mean titer; OPA = opsonophagocytic activity.
^{a}n = Number of subjects with a determinate OPA titer to the given serotype.
^{b}GMTs were calculated using all subjects with available data for the specified blood draw.
^{c}CIs are back transformations of CIs based on the Student t distribution for the mean logarithm of the titers.
^{d}Ratio of GMTs (20vPnC/Saline to 13vPnC/PPSV23) was calculated by back transforming the mean difference in GMTs between vaccine sequences on the logarithmic scale.
^{e}CIs for the ratio are back transformations of CIs based on the Student t distribution for the mean difference of the logarithms of the measurements (20vPnC/Saline minus 13vPnC/PPSV23).

### CONCLUSION

Well characterized OPAs were developed for two Streptococcus pneumoniae serotype 15A strains and two S. pneumoniae serotype 15C strains. Serum from subjects immunized with 20vPnC, a 15B capsular polysaccharide-containing vaccine, elicited cross-functional OPA activity against both 15C strains. This cross-functional activity was confirmed with a larger sample set against a single 15C strain. Serum from subjects immunized with 20vPnC elicited some cross-functional activity against the S. pneumoniae 15A strains tested, although the titers were low.

All publications and patent applications mentioned in the specification are indicative of the level of those skilled in the art to which this invention pertains. All publications and patent applications are hereby incorporated by reference to the same extent as if each individual publication or patent application was specifically and individually indicated to be incorporated by reference.

Although the foregoing invention has been described in some detail by way of illustration and example for purposes of clarity of understanding, certain changes and modifications may be practiced within the scope of the appended claims.

1. A set of immunogenic compositions comprising: (a) a first immunogenic composition comprising a glycoconjugate from S. pneumoniae serotype 15B; and (b) a second immunogenic composition comprising a glycoconjugate from S. pneumoniae serotype 15A and a glycoconjugate from S. pneumoniae serotype 15C, wherein said compositions are for concurrent, concomitant or sequential administration.

2. A set of immunogenic compositions comprising: (a) a first immunogenic composition comprising a glycoconjugate from S. pneumoniae serotype 15B; and (b) a second immunogenic composition comprising a glycoconjugate from S. pneumoniae serotype 15A, wherein said compositions do not comprise capsular saccharide from S. pneumoniae serotypes 15C and wherein said compositions are for concurrent, concomitant or sequential administration.

3. The set of immunogenic compositions of claim 1, wherein said first immunogenic composition further comprises, in addition to a glycoconjugate from S. pneumoniae serotype 15B, glycoconjugates from S. pneumoniae serotypes 1, 3, 4, 5, 6A, 6B, 7F, 8, 9V, 10A, 11A, 12F, 14, 18C, 19A, 19F, 22F, 23F and 33F.

4. The set of immunogenic compositions of claim 1, wherein the glycoconjugates of the first immunogenic compositions are all individually conjugated to CRM₁₉₇.

5. The set of immunogenic compositions of claim 1, wherein the first immunogenic composition comprises from 7 to 25 different serotypes of S. pneumoniae.

6. The set of immunogenic compositions of claim 1, wherein, the first immunogenic composition is a 20-valent pneumococcal conjugate composition wherein in addition to a glycoconjugate from S. pneumoniae serotype 15B, said composition further comprises, glycoconjugates from S. pneumoniae serotypes 1, 3, 4, 5, 6A, 6B, 7F, 8, 9V, 10A, 11A, 12F, 14, 18C, 19A, 19F, 23F, 22F and 33F individually conjugated to CRM₁₉₇.

7. The set of immunogenic compositions of claim 1, wherein the second immunogenic composition comprises from 7 to 25 different serotypes of S. pneumoniae.

8. An immunogenic composition comprising a glycoconjugate from S. pneumoniae serotype 15A wherein said immunogenic composition does not comprise glycoconjugates from S. pneumoniae serotypes 4, 6B, 9V, 14, 15B, 15C, 18C, 19F and 23F, for use in a method of complementing a pneumococcal conjugate vaccine which comprises a glycoconjugate from S. pneumoniae serotype 15B but which does not comprise glycoconjugates from S. pneumoniae serotypes 15A.

9. The immunogenic composition for use of claim 8 wherein said immunogenic composition does not comprise glycoconjugates from S. pneumoniae serotypes 1, 4, 5, 6B, 7F, 9V, 14, 15B, 15C, 18C, 19F and 23F.

10. The immunogenic composition for use of claim 8, comprising glycoconjugates from 2 to 25 different serotypes of S. pneumoniae.

11. The immunogenic composition for use of claim 8, which is a 2 to 24-valent pneumococcal conjugate composition.

12. A kit comprising the set of immunogenic compositions of claim 1, and an information leaflet.


SEQUENCE LISTING (10 sequences)

SEQ ID NO: 1 - DNA, 21 bp, Artificial Sequence
  “B class” CpG oligonucleotide
tcgtcgtttttcggtgctttt

SEQ ID NO: 2 - DNA, 21 bp, Artificial Sequence
  “B class” CpG oligonucleotide
tcgtcgtttttcggtcgtttt

SEQ ID NO: 3 - DNA, 24 bp, Artificial Sequence
  “B class” CpG oligonucleotide
tcgtcgttttgtcgttttgtcgtt

SEQ ID NO: 4 - DNA, 24 bp, Artificial Sequence
  “B class” CpG oligonucleotide
tcgtcgtttcgtcgttttgtcgtt

SEQ ID NO: 5 - DNA, 24 bp, Artificial Sequence
  “B class” CpG oligonucleotide
tcgtcgttttgtcgtttttttcga

SEQ ID NO: 6 - DNA, 21 bp, Artificial Sequence
  “B class” CpG oligonucleotide
tcgtcgtttttcggtgctttt

SEQ ID NO: 7 - DNA, 21 bp, Artificial Sequence
  “B class” CpG oligonucleotide
tcgtcgtttttcggtcgtttt

SEQ ID NO: 8 - DNA, 24 bp, Artificial Sequence
  “B class” CpG oligonucleotide
tcgtcgttttgtcgttttgtcgtt

SEQ ID NO: 9 - DNA, 24 bp, Artificial Sequence
  “B class” CpG oligonucleotide
tcgtcgtttcgtcgttttgtcgtt

SEQ ID NO: 10 - DNA, 24 bp, Artificial Sequence
  “B class” CpG oligonucleotide
tcgtcgttttgtcgtttttttcga